(19) United States
(12) Patent Application Publication
(10) Pub. No.: US 2003/0187027 A1
(43) Pub. Date: Oct. 2, 2003

(54) Dioxanes and uses thereof

(76) Inventors: Stuart L. Schreiber, Boston, MA (US); Scott M. Sternson, New York, NY (US); Jason C. Wong, Cambridge, MA (US); Christina M. Grozinger, Champagne, IL (US)

Correspondence Address: Choate, Hall & Stewart, Exchange Place, 53 State Street, Boston, MA, 02109, US

(21) Appl. No.: 10/144,316

(22) Filed: May 9, 2002

Related U.S. Application Data
(60) Provisional application No. 60/289,850, filed on May 9, 2001.

(51) Int. Cl. A61K 31/4433; C07D 319/06; A61K 31/335; C 07D 4 5/02

(52) U.S. Cl. 514/336; 514/452; 546/282.4; 549/369; 549/370

(57) ABSTRACT

In recognition of the need to develop novel therapeutic agents and efficient methods for the synthesis thereof, the present invention provides novel compounds of general formula (I):

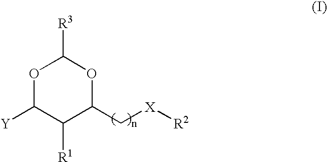

and pharmaceutically acceptable derivatives thereof, wherein R¹, R², R³, n, X and Y are as defined herein. The present invention also provides pharmaceutical compositions comprising a compound of formula (I) and a pharmaceutically acceptable carrier. The present invention further provides compounds capable of inhibiting histone deacetylatase activity and methods for treating disorders regulated by histone deacetylase activity (e.g., cancer and protozoal infections) comprising administering a therapeutically effective amount of a compound of formula (1) to a subject in need thereof. The present invention additionally provides methods for modulating the glucose-sensitive subset of genes downstream of Ure2p.

### PORITY INFORMATION

The present application claims priority under 35 U.S.C. § 119(e) to provisional application number 60/289,850, filed May 9, 2001, entitled “HDAC Inhibitors”, the entire contents of which are hereby incorporated by reference.

### GOVERNMENT SUPPORT

This invention was made in part with a grant from the National Institutes of Health (Grant Number: GM38627). Therefore, the government has certain rights in the invention.

### BACKGROUND OF THE INVENTION

The identification of small organic molecules that affect specific biological functions is an endeavor that impacts both biology and medicine. Such molecules are useful as therapeutic agents and as probes of biological function. In but one example from the emerging field of chemical genetics, in which small molecules can be used to alter the function of biological molecules to which they bind, these molecules have been useful at elucidating signal transduction pathways by acting as chemical protein knockouts, thereby causing a loss of protein function. (Schreiber et al., J. Am. Chem. Soc., 1990, 112, 5583; Mitchison, Chem. and Biol., 1994, 1, 3) Additionally, due to the interaction of these small molecules with particular biological targets and their ability to affect specific biological function, they may also serve as candidates for the development of therapeutics. One important class of small molecules, natural products, which are small molecules obtained from nature, clearly have played an important role in the development of biology and medicine, serving as pharmaceutical leads, drugs (Newman et al., Nat. Prod. Rep. 2000, 17, 215-234), and powerful reagents for studying cell biology (Schreiber, S. L. Chem. and Eng. News 1992 (October 26), 22-32).

Because it is difficult to predict which small molecules will interact with a biological target, and it is oftent difficult to obtain and synthesize efficiently small molecules found in nature, intense efforts have been directed towards the generation of large numbers, or libraries, of small organic compounds, often “natural product-like” libraries. These libraries can then be linked to sensitive screens for a particular biological target of interest to identify the active molecules.

One biological target of recent interest is histone deacetylase (see, for example, a discussion of the use of inhibitors of histone deacetylases for the treatment of cancer: Marks et al. Nature Reviews Cancer 2001, 1,194; Johnstone et al. Nature Reviews Drug Discovery 2002, 1, 287). Post-translational modification of proteins through acetylation and deacetylation of lysine residues has a critical role in regulating their cellular finctions. HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). HDACs participate in cellular pathways that control cell shape and differentiation, and an HDAC inhibitor has been shown effective in treating an otherwise recalcitrant cancer (Warrell et al. J. Natl. Cancer Inst. 1998, 90, 1621-1625). Nine human HDACs have been characterized ((a) Taunton et al. Science 1996, 272, 408-411; (b) Yang et al. J. Biol. Chem. 1997, 272, 28001-28007. (c) Grozinger et al. Proc. Natl. Acad. Sci. U.S.A. 1999, 96, 4868-4873. (d) Kao et al. Genes Dev. 2000, 14, 55-66. (e) Hu et al. J. Biol. Chem. 2000, 275, 15254-15264. (f) Zhou et al. Proc. Natl. Acad. Sci. U.S.A. 2001, 98, 10572-10577) and two inferred (Venter et al. Science 2001, 291, 1304-1351) these members fall into two related classes (class I and II). To date, no small molecules are known that selectively target either the two classes or individual members of this family ((for example ortholog-selective HDAC inhibitors have been reported: (a) Meinke et al. J. Med. Chem. 2000, 14, 4919-4922; (b) Meinke, et al. Curr. Med. Chem. 2001,8,211-235).

Clearly, it would be desirable to develop compounds capable of selectively targeting either of the two classes or individual members of this family. Additionally, it would be desirable to identify novel compounds capable of acting as probes of biological finction.

### SUMMARY OF THE INVENTION

As discussed above, there remains a need for the development of novel therapeutic agents and agents capable of elucidating biological functions. The present invention provides novel compounds of general formula (I),

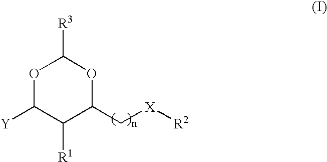

and pharmaceutical compositions thereof, as described generally and in subclasses herein, which compounds are useful as inhibitors of histone deacetylases, and thus are useful for the treatment of proliferative diseases and as antiprotozoal agents. The inventive compounds are additionally useful as tools to probe biological function.

### DESCRIPTION OF THE FIGURES

FIG. 1 depicts sequence comparison of residues on the rim of the N-acetyl lysine binding channel. Amino acids in HDLP that contact TSA are boxed in gray. The numbering is based on the HDLP sequence.

FIG. 2 depicts a scheme for synthesis of compounds of the invention.

FIG. 3 depicts exemplary inhbitors of HDAC 1 and HDAC6.

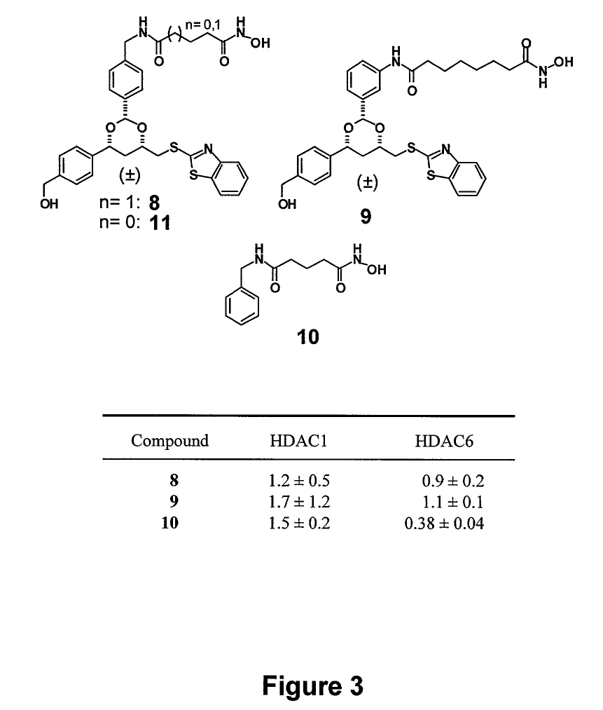

FIG. 4 depicts the synthesis of linker S3.

FIG. 5 depicts Mosher ester derivitization.

FIG. 6 depicts the synthesis of epoxyol S15.

FIG. 7 depicts the synthesis of exemplary reagents S32, S33 and S34.

FIG. 8 depicts exemplary building blocks used for a 7200 member 1,3-dioxane library.

FIG. 9 depicts (a) selection of nucleophilic building blocks; (b) selection of Fmoc-amino dimethyl acetal building blocks; and (c) selection of diacid building blocks.

FIG. 10 depicts anti-acetyl-lysine 40 tubulin and anti-acetyl-histone immunofluorescence BS-C-1 cells (14 h treatment).

FIG. 11 depicts anti-acetyl-lysine 40 tubulin and anti-acetyl-histone immunofluorescence BS-C-1 cells (14 h treatment).

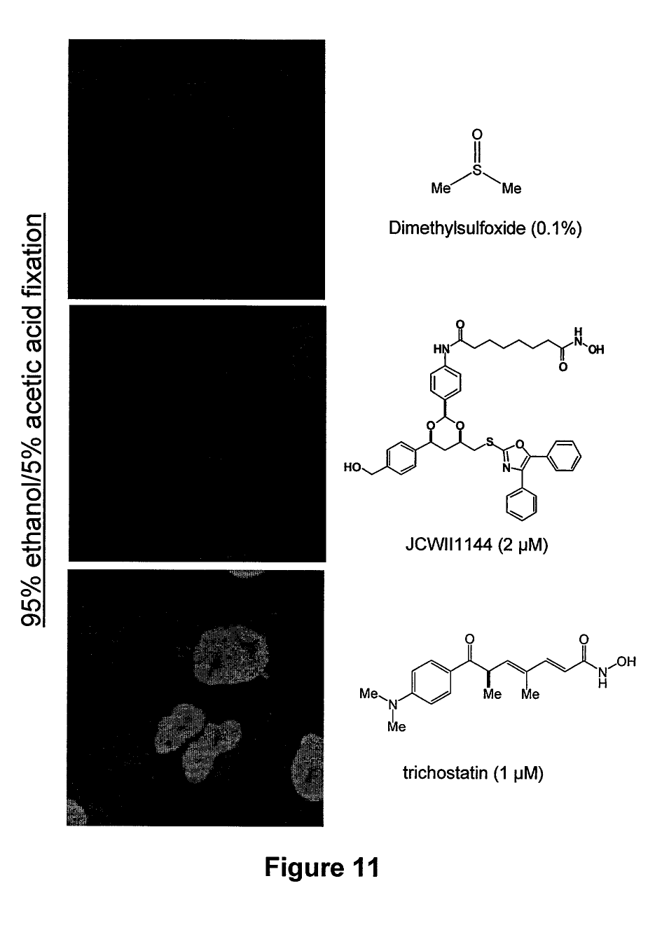

FIG. 12 depicts the effect of an inventive compound on acetylated tubulin and acetylated histone H3 in A549 cells (5 h treatment).

FIG. 13 depicts exemplary concentration response curves for inhibition of HDAC1, 4and6.

FIG. 14 depicts the effect of an inventive compound on acetylated tubulin and acetylated histone H3 in A549 cells (5 h treatment).

FIG. 15 depicts the effect of an inventive compound on acetylated tubulin levels in A549 cells (18 h treatment).

FIG. 16 depicts the effect of an inventive compound on total acetylated lysine levels in A549 cells (18 h treatment).

FIG. 17 depicts exemplary enantiomers and their potency and selectivity.

FIG. 18 depicts synthesis of an exemplary solid support unit (solid support and linker).

FIG. 19 depicts an exemplary synethetic scheme.

FIG. 20 depicts exemplary (a) epoxy alcohol building blocks, (b) amine and thiol building blocks, (c) Fmoc-amino dimethylacetal building blocks and (d) electrophile building blocks.

FIG. 21 depicts structure determination procedure for the 1,3-dioxane library. (a) from LC-MS: UV absorbance trace, total ion count (TIC) trace (APCI+) and mass spectrum under the major peak. The molecular ion (M+1) is 679 amu. (b) Determination of precursor amine mass. (c) All possible combinations of epoxyalcohol, nucleophile, and acetal building block masses. The mass being referenced, 564 amu results from two possible combinations of building blocks represented in the two possible structures. Fragments with masses of 429 and 411 amu are consistent only with structure 8. (e) Sample from the synthesis of the proposed structure 8 (trace A) shows the same retention time as a mixture of the synthesized compound and a sample from the original stock solution (trace B).

FIG. 22 depicts molecules identified to show activity in phenotypic and protein-binding assays: (a) molecules showing activity in a variety of phenotypic assays in Xenopuslaevis extract and in HeLa cells; (b) 1,3-diol 13 causes a wavy notochord phenotype (arrow) in Zebrafish embryos 24 h post fertilization; and (c) FKBP12 ligand identified using a small molecule microarray (a magnified portion of the array is shown).

FIG. 23 depicts depicts the library synthesis and identification of uretupamine. (a) Outline of the diversity-oriented synthesis leading to uretupamine and other library members. (b) An expanded view of 64 compound spots on the 3,780-member small molecule microarray (˜800 spots cm⁻²). Cy5 labelled Ure2p was passed over a microarray of the 1,3-dioxane small molecule library, and the resulting slide was washed three times and scanned for fluorescence. The spot corresponding to uretupamine A is shown.

FIG. 24 depicts studies in vivo, dose response and structure-activity relationships of uretupamine. (a) A yeast strain (DB26-3A) growing in YPD medium expressing a PUT1-lacZ reporter was treated with 50 nM rapamycin or with a compound that had been detected to bind to labelled Ure2p on a small-molecule microarray. After 90 min of treatment at 30 C, a standard liquid b-galactosidase assay was performed. Data are expressed in fold Miller units compared with treatment with 50 nM rapamycin for 90 minutes. DB26-3A (MAT-a ura3-52 ade2 PUT1-lacZ) was a gift from M. Brandriss. Vehicle: samples treated with N,N-dimethylformamide (DMF), the vehicle into which library compounds were dissolved (b): Uretupamine A was resynthesized and tested in the b-galactosidase assay by using the PUT1-lacZ reporter at the concentrations indicated for 60 min at 30 C in YPD medium. (c) Compounds derived from the uretupamine A structure were synthesized to explore structure-activity relationships. Listed are b-galactosidase assay results of treatment with each compound at 100 μM (asterisk designates 50 μM) for 60 min at 30 C in YPD medium. Data are in percentage Miller units compared with treating with 50 nM rapamycin for 60 min. Ac, acetate; MDPO, 2-mercapto-4,5-diphenyloxazole; MBO, 2-mercaptobenzoxazole; Ph, phenyl. (d) Binding of uretupamine B to Ure2p was determined by using surface plasmon resonance (BlAcore 3000) to have a dissociation constant of 7.5 μM. Data points were acquired in triplicate. Ure2p was immobilized to CM5 sensor chips by injection of 100 μg/ml Ure2p in 10 mM sodium acetate pH 4.5 in accordance with the manufacturer's procedures. The reference cell was derivatized with antibodies against glutathione S-transferase (GST) followed by GST capture. Small-molecule binding measurements and dissociation were in PBS/Tween-20 containing 10% DMF flow rate 5 μl/min).

FIG. 25 depicts transcription profiling of treatment with uretupamine. (a) The left microarray corresponds to wild-tyupe cells (PM38) treated with vehicle (DMF) versus wild type (w.t.) cells treated for 30 min with uretupamine A at 100 μM. The right microarray corresponds to ure2Δ cells (PH2) treated with vehicle versus ure2Δ cells treated for 30 min with uretupamine A at 100 μM. Profiles were obtained as described (Hardwick et al. Proc. Natl. Acad. Sci. USA 1999, 96, 14866-14870). At the right are shown specific gene inductions of some URE2-dependent genes from the microarrays. PM38 (MATaleu2-3,112 ura3-52), PM71 (MATaleu2-3,112 ura3-52 gln3Δ5::LEU2), MS221 (MATaura3-52 nill::hisG), PH2 (MATaleu2-3, 112 ura3-52 ure2Δ12:: URA3) were gifts from B. Magasanik and M. Brandriss. (b) The transcription profiles of wild-type cells (PM38), ure2Δ cells (PH2), gln3Δ cells (PM71) and nil1Δ cells (MS221) grown in YPD medium treated for 30 min with 100 μM uretupamine B were obtained. The geometric means of gene inductions of the sets listed are shown. (c) An analysis was performed based on treating individual profiles as high-dimensional vectors and then examining the ratios of their magnitudes as a measure of relative activity (Shamji et al. Curr. Biol. 2000, 10, 1574-1581; Kuruvilla et al. Genome Biol. 2002, 3(3), 0011.1-0011.11).

FIG. 26 depicts glucose-sensitive signalling and a model of Ure2p function. (a): A wild type strain (PM38) was grown to mid-exponential phase in synthetic glucose (dextrose) (SD)-ammonium sulphate (AS) medium, washed with PBS and split into either SD-AS medium, SD-AS medium containing 50 nM rapamycin, SD-proline medium, synthetic acetate-AS medium or synthetic glycerol-AS medium, then incubated with shaking for 1 h at 30° C. Control: the sample split into SD-AS medium. Synthetic medium consisted of 1.7 g of YNB medium, without amino acids and without AS, 2% carbon sourced, 0.1% nitrogen source and auxotrophic supplements when needed (leucine 120 mg I⁻¹, uracil 20 mg I⁻¹). Whole cell lysates were blotted with anti-Ure2p antibodies kindly provided by R. Wickner as described previously. A previous report claimed that Ure2p dephosphorylation does occur upon nitrogen limitation but those authors examined cells shifted from a rich medium to a synthetic-nitrogen-limited medium, thus changing many variables of the medium simultaneously. In experiments conducted herein, cells were shifted from a synthetic medium containing 0.1% ammonium sulphate as a high quality nitrogen source to an otherwise identical medium containing 0.1% proline as a low-quality nitrogen source. Inspection of published Ure2p immunoblots under similar conditions also supports the lack of a mobility shift (see, Edskes et al. Genetics 1999, 153, 585-594; Edskes et al. Proc. Natl. Acad. Sci. US.A. 2000, 97, 6625-6629). (b): A wild-type strain (PM38) was treated for 1 h with no drug, 50 nM rapamycin, salt stress (1M NaCl), high osmolarity (1M sorbitol) or high pH (pH 9.5). (c) A wild-type strain (PM38) was shifted from an SD-AS medium to a synthetic acetate—AS medium and incubated with shaking for 30 min at 30° C. This transcription profile was compared to the profile of the same strain shifted from SD-AS to synthetic ethanol-AS medium. The expression of various subsets of genes was compared between profiles by using vector algebra and the results are presented in a calorimetric comparison array. (d) Inspection of Ure2p-dependent genes reveals that uretupamine-sensitive genes behave similarly when cells are shifted to ethanol or acetate, whereas uretupamine-insensitive genes behave differently. (e) Nitrogen quality regulates the Ure2p-Gln3p/Nillp complex by signalling to Gln3p/Nillp, whereas low glucose concentration regulates the complex by signalling to Ure2p. Low glucose concentration leads to dephosphorylation of Ure2p to induce a specific set of genes involved in an anaplerotic sequence, a state mimicked by uretupamine.

### DETAILED DESCRIPTION OF THE INVENTION

As discussed above, there remains a need for the development of novel therapeutic agents and agents capable of elucidating biological functions. The present invention provides novel compounds of general formula (I), and methods for the synthesis thereof, which compounds are useful as inhibitors of histone deacetylases, and thus are useful for the treatment of proliferative diseases and as antiprotozoal agents. The inventive compounds are additionally useful as tools to probe biological function.

1) General Description of Compounds of the Invention

In general, the present invention provides compounds having the general structure (I):

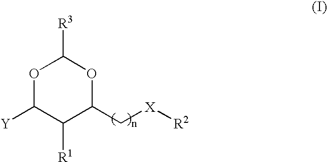

and pharmaceutically acceptable derivatives thereof,

wherein R¹ is hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety;

n is 1-5;

R² is hydrogen, a protecting group, or an aliphatic, heteroaliphatic, aryl, heteroaryl, - (aliphatic)aryl, -(aliphatic)heteroaryl, -(heteroaliphatic)aryl, or -(heteroaliphatic)heteroaryl moiety;

X is —O—, —C(R^{2A})₂—, —S—, or —NR^{2A}—, wherein R^{2A} is hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety;

or wherein two or more occurrences of R² and R^{2A}, taken together, form a cyclic aliphatic or heteroaliphatic moiety, or an aryl or heteroaryl moiety;

R³ is an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety; and

Y is hydrogen or an aliphatic, heteroaliphatic, aryl, heteroaryl, -(aliphatic)aryl, - (aliphatic)heteroaryl, -(heteroaliphatic)aryl, or -(heteroaliphatic)heteroaryl moiety,

whereby each of the foregoing aliphatic and heteroaliphatic groups is independently substituted or unsubstituted, cyclic or acyclic, linear or branched, and each of the foreoing aryl and heteroaryl groups is substituted or unsubstituted.

In certain embodiments of the invention, compounds of formula (1) have the following stereochemistry and structure as shown in formula (Ia):

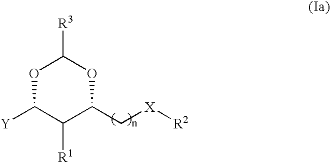

It will be appreciated that, in certain embodiments of the compounds as described generally above and in classes and subclasses herein certain classes of compounds are excluded:

compounds where X is NR^{2A} and Y is a phenyl group substituted with —(C═O)NH₂ or —(C═O)NH(solid support).

2) Featured Classes of Compounds

It will be appreciated that for compounds as generally described above, certain classes of compounds are of special interest.

For example, one class of compounds of special interest includes those compounds of the invention as described above and in certain subclasses herein, in which Y is an aryl or heteroaryl moiety substituted with Z, wherein Z is hydrogen, —(CH₂)_{q}OR^{Z}, —(CH₂)_{q}SR^{Z}, —(CH₂)_{q}N(R^{Z})₂, —(C═O)R^{Z}, —(C═O)N(R^{Z})₂, or an aliphatic, heteroaliphatic, aryl, heteroaryl, —(aliphatic)aryl, —(aliphatic)heteroaryl, —(heteroaliphatic)aryl, or —(heteroaliphatic)heteroaryl moiety, wherein q is 0-4, and wherein each occurrence of R^{Z} is independently hydrogen, a protecting group, a solid support unit, or an aliphatic, heteroaliphatic, aryl, heteroaryl, —(aliphatic)aryl, —(aliphatic)heteroaryl, —(heteroaliphatic)aryl, or —(heteroaliphatic)heteroaryl moiety.

Another class of compounds of special interest includes those compounds of the invention as described above and in certain subclasses herein, wherein the compounds have the general structure (II) in which Y is a substituted phenyl moiety as depicted below:

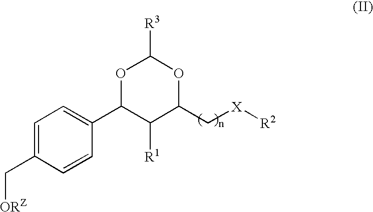

Yet another class of compounds of special interest includes those compounds of the invention as described above and in certain subclasses herein, wherein the compounds have the general structure (III) in which Y is a substituted phenyl moiety and X is S:

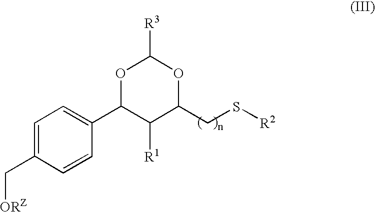

Yet another class of compounds of special interest includes those compounds of the invention as described above and in certain subclasses herein, wherein the compounds have the general structure (IV) in which Y is a substituted phenyl moiety and X is —NR^{2A}—:

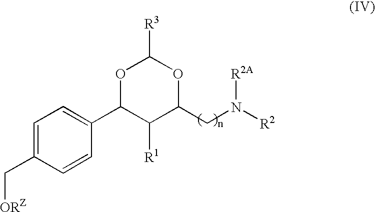

Yet another class of compounds of special interest includes those compounds of the invention as described above and in certain subclasses herein, wherein the compounds have the general structure (V) in which Y is a substituted phenyl moiety and X is O:

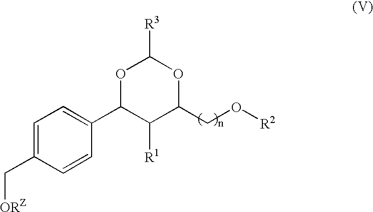

Still another class of compounds of special interest includes those compounds of the invention as described above and in certain subclasses herein, wherein the compounds have the general structure (VI) in which Y is a substituted phenyl moiety and R³ is a phenyl moiety substituted with R⁴:

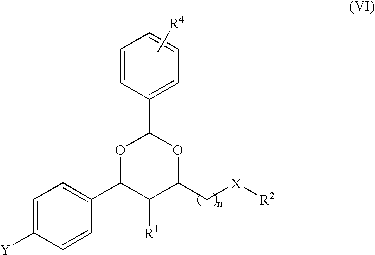

wherein R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, —(aliphatic)aryl, —(aliphatic)heteroaryl, —(heteroalipahtic)aryl, or —(heteroaliphatic)heteroaryl moiety, wherein each occurrence of R^{4A} is independently hydrogen, a protecting group, an aliphatic, heteroaliphatic, aryl, heteroaryl, —(aliphatic)aryl, —(aliphatic)heteroaryl, —(heteroalipahtic)aryl, or —(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, —(aliphatic)aryl, —(aliphatic)heteroaryl, —(heteroalipahtic)aryl, or —(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted.

Yet another class of compounds of special interest includes those compounds of the invention as described above and in certain subclasses herein, wherein the compounds have the general structure (VII) in which Y is a substituted phenyl moiety and R³ is a phenyl moiety substituted with R⁴:

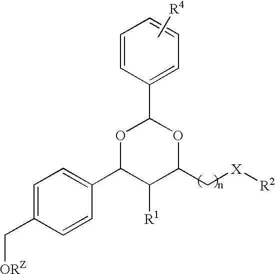

wherein R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, —(aliphatic)aryl, —(aliphatic)heteroaryl, —(heteroalipahtic)aryl, or —(heteroaliphatic)heteroaryl moiety, wherein each occurrence of R^{4A} is independently hydrogen, a protecting group, an aliphatic, heteroaliphatic, aryl, heteroaryl, —(aliphatic)aryl, —(aliphatic)heteroaryl, —(heteroalipahtic)aryl, or —(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted.

The following compounds are illustrative of certain of the compounds described generally and in classes and subclasses herein:

A number of important subclasses of each of the foregoing classes deserve separate mention; these subclasses include subclasses of each of the foregoing classes in which:

i) compounds of the invention as described above and herein wherein R¹ is hydrogen, lower alkyl, or a substituted or unsubstituted phenyl moiety;

ii) compounds of the invention as described above and herein wherein R¹ is hydrogen, methyl, or phenyl;

iii) compounds of the invention as described above and herein wherein R¹ is hydrogen;

iv) compounds of the invention as described above and herein wherein either or both of R², R^{2A} or R² and R^{2A}, taken together with N, comprise

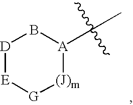

wherein m is 0-3; A-B, B-D, D-E, E-G, G-J, two or more occurrences of J, and J-A are each connected by a single or double bond; A is CH, C, or N; B is CR^{B}, C(R^{B})₂, (C═O), NR^{B}, N, O or S; D is CR^{D}, C(R^{D})₂, (C═O), NR^{D}, N, O or S; E is CR^{E}, C(R^{E})₂, (C═O), NR^{E}, N, O or S; G is CR^{G}, C(R^{G})₂, (C═O), NR^{G}, N, O or S; and each occurrence of J is independently CR^{J}, C(R^{J})₂, (C═O), NR^{J}, N, O or S;

wherein each occurrence of R^{B}, R^{D}, R^{E}, R^{G} and R^{J} is independently hydrogen, halogen, hydroxyl, protected hydroxyl, thiol, protected thiol, amino, protected amino, —COOH, —CONH₂, —NHCOOH, —NHCOO(alkyl), —NHCO(alkyl), or a substituted or unsubstituted, cyclic or acyclic, linear or branched alkyl or heteroalkyl moiety, or a substituted or unsubstituted aryl or heteroaryl moiety, or any two or R^{B}, R^{D}, R^{E}, R^{G} or R^{J} taken together comprises a substituted or unsubstituted cyclic aliphatic or heteroaliphatic, moiety or a substituted or unsubstituted aryl or heteroaryl moiety;

v) compounds as described above and herein wherein —X—R² has one of the structures:

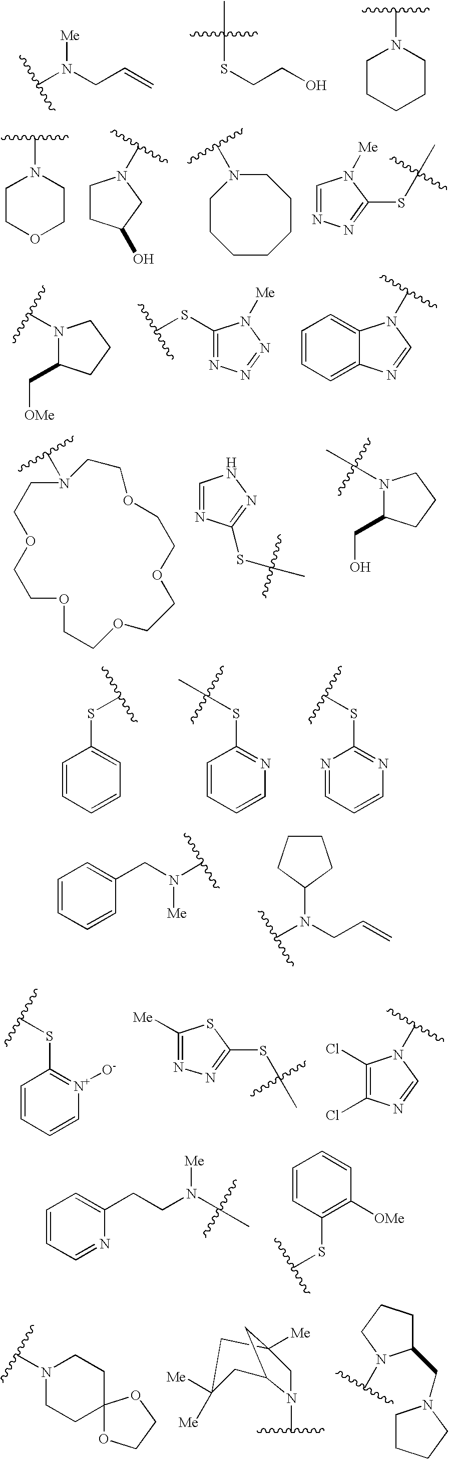

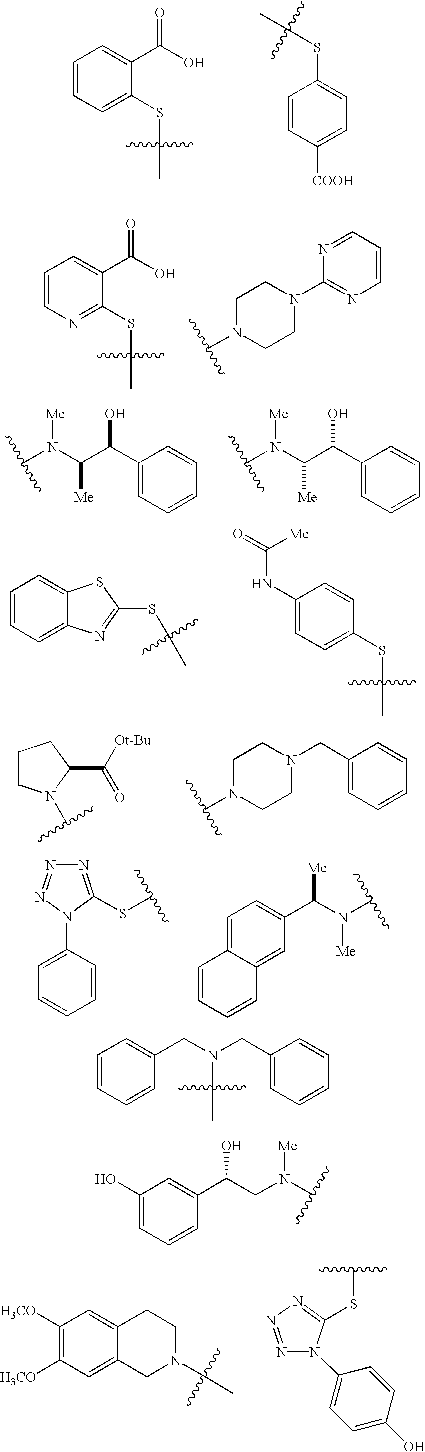

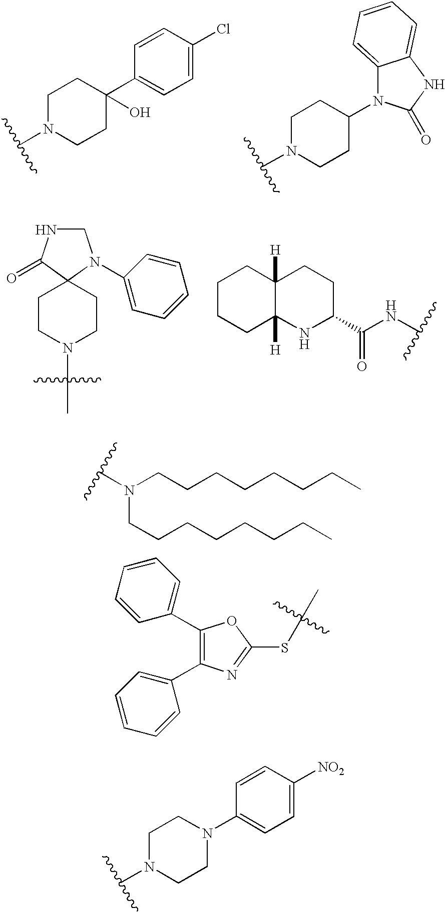

vi) compounds of the invention as described above and herein wherein one or both of R² and R^{2A} is a substituted or unsubstituted aryl or heteroaryl moiety;

vii) compounds of the invention as described above and herein wherein one or both of R² and R^{2A} is an aryl or heteroaryl moiety substituted with —COOH, halogen, alkyl, heteroalkyl, aryl, heteroaryl, OH, SH, NO₂, NH₂, or —NH(C═O)alkyl;

viii) compounds of the invention as described above and herein wherein R³ is a substituted aryl or heteroaryl moiety;

ix) compounds of the invention as described above and herein wherein R³is an aryl or heteroaryl moiety substituted with —CH₂)ᵣN(R^{4A})₂, wherein r is 0 or 1 and each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted;

x) compounds of the invention as described above and herein wherein R³ is one of the following structures:

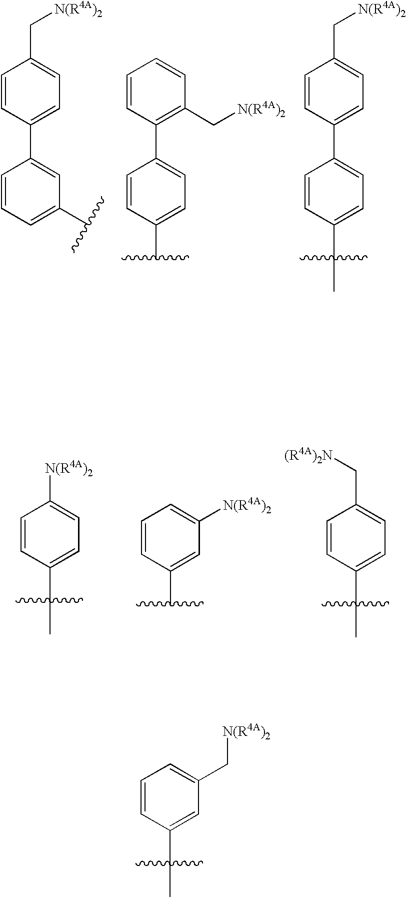

xi) compounds of the invention as described above and herein wherein R^{Z} is hydrogen or a solid support unit; and

xii) compounds of the invention as described above and herein wherein R⁴ is —(C═O)(CH₂)ₜ(C═O)NHR^{4B}, wherein R^{4B} hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted;

xiii) compounds of the invention as described above and herein wherein R^{4A} is —(C═O)(CH₂)ₜ(C═O)NHR^{4B}, wherein R^{4B} is hydroxyl and t is 3, 4 or 5;

xiv) compounds of the invention as described above and herein wherein R^{4A} is —(C═O)(CH₂)ₜ(C═O)NHR^{4B}, wherein R^{4B} is hydroxyl, t is 3, 4 or 5, and X—R² is —S—R².

As the reader will appreciate, compounds of particular interest include, among others, those which share the attributes of one or more of the foregoing subclasses. Some of those subclasses are illustrated by the following sorts of compounds:

I) Compounds of the formula:

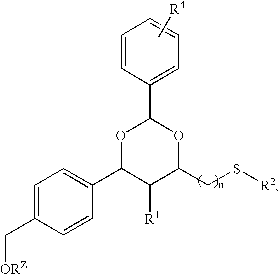

wherein R¹, R², R⁴, n and R^{Z} are as described in classes and subclasses herein.

In certain exemplary embodiments, R¹ is hydrogen, phenyl or methyl, R^{Z} is hydrogen or a solid support unit; R² is a substituted or unsubstituted alkyl or heteroalkyl moiety, or a substituted or unsubstituted aryl or heteroaryl moiety; and R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, —(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted.

In certain exemplary embodiments, R^{4A} is —(C═O)(CH₂)ₜ(C═O)NHR^{4B}, wherein R^{4B} is hydroxyl, t is 3, 4 or 5.

II) Compounds of the formula:

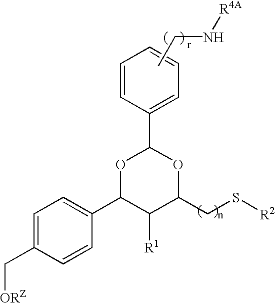

wherein R¹, R², R⁴, n and R^{Z} are as described in classes and subclasses herein.

In certain exemplary embodiments, R¹ is hydrogen, phenyl or methyl, R^{Z} is hydrogen or a solid support unit; R² is a substituted or unsubstituted alkyl or heteroalkyl moiety, or a substituted or unsubstituted aryl or heteroaryl moiety; and R^{4A} A —(C═O)(CH₂)ₜ(C═O)NHR^{4B}, wherein R^{4B} is hydroxyl, t is 3, 4 or 5.

III) Compounds of the formula:

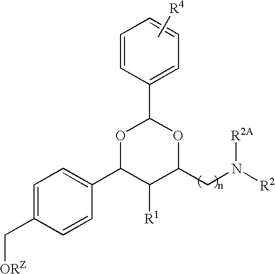

wherein R¹, R², R^{2A}, R⁴, n and R^{Z} are as described in classes and subclasses herein.

In certain exemplary embodiments, R¹ is hydrogen, phenyl or methyl, R^{Z} is hydrogen or a solid support unit; either or both of R² and R^{2A}, or R² and R^{2A} taken together with N, is a substituted or unsubstituted alkyl or heteroalkyl moiety, or a substituted or unsubstituted aryl or heteroaryl moiety; and R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B})₂, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,

whereby each of the foregoing aliphatic and heteroaliphatic groups is independently substituted or unsubstituted, cyclic or acyclic, linear or branched, and each of the foreoing aryl and heteroaryl groups is substituted or unsubstituted.

In certain exemplary embodiments, R^{4A} is —(C═O)(CH₂)ₜ(C═O)NHR^{4B}, wherein R^{4B} is hydroxyl, t is 3, 4 or 5.

IV) Compounds of the formula:

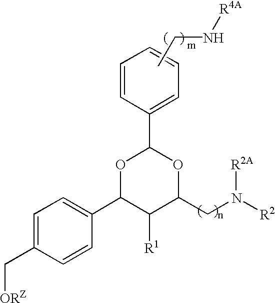

wherein R¹, R², R^{2A}, R⁴, n and R^{Z} are as described in classes and subclasses herein.

In certain exemplary embodiments, R¹ is hydrogen, phenyl or methyl, R^{Z} is hydrogen or a solid support unit; either or both of R² and R^{2A}, or R² and R^{2A} taken together with N, is a substituted or unsubstituted alkyl or heteroalkyl moiety, or a substituted or unsubstituted aryl or heteroaryl moiety; and R^{4A} is —(C═O)(CH₂)ₜ(C═O)NHR^{4B}, wherein R^{4B} is hydroxyl, t is 3, 4 or 5.

It will be appreciated that some of the foregoing classes and subclasses of compounds can exist in various isomeric forms. The invention encompasses the compounds as individual isomers substantially free of other isomers and alternatively, as mixtures of various isomers, e.g., racemic mixtures of stereoisomers. Additionally, the invention encompasses both (Z) and (E) double bond isomers unless otherwise specifically designated. The invention also encompasses tautomers of specific compounds as described above. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which:

exhibit HDAC-inhibitory activity;

exhibit the ability to inhibit HDAC1;

exhibit the ability to inhibit HDAC6;

exbhit the ability to modulate the glucose-sensitive subset of genes downstream of Ure2p;

exhibit cytotoxic or growth inhibitory effect on cancer cell lines maintained in vitro or in animal studies using a scientifically acceptable cancer cell xenograft model;

exhibit a therapeutic profile (e.g., optimum safety and curative effect) that is superior to existing chemotherapeutic agents.

This invention also provides a pharmaceutical preparation comprising at least one of the compounds as described above and herein, or a pharmaceutically acceptable derivative thereof, which compounds are capable of inhibiting the growth of or killing cancer cells. In certain other embodiments, the compounds are useful for the treatment of protozoal infections.

The invention further provides a method for inhibiting tumor growth and/or tumor metastasis. The method involves the administration of a therapeutically effective amount of the compound or a pharmaceutically acceptable derivative thereof to a subject (including, but not limited to a human or animal) in need of it. In certain embodiments, specifically for treating cancers comprising multidrug resistant cancer cells, the therapeutically effective amount is an amount sufficient to kill or inhibit the growth of multidrug resistant cancer cell lines. In certain embodiments, the inventive compounds are useful for the treatment of solid tumors.

As discussed above, the compounds of the invention are selective inhibitors of histone deacetylases and, as such, are useful in the treatment of disorders modulated by histone deacetylases.

3) Compounds and Definitions

As discussed above, the present invention provides a novel class of compounds useful for the treatment of cancer and other proliferative conditions related thereto. Compounds of this invention comprise those, as set forth above and described herein, and are illustrated in part by the various classes, subgenera and species disclosed elsewhere herein.

It will be appreciated by one of ordinary skill in the art that numerous asymmetric centers exist in the compounds of the present invention. Thus, inventive compounds and pharmaceutical compositions thereof may be in the form of an individual enantiomer, diastereomer or geometric isomer, or may be in the form of a mixture of stereoisomers.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., C, O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are Eds., John Wiley & Sons, New York: 1999, the entire contents of which are hereby incorporated by reference. Furthermore, a variety of carbon protecting groups are described in Myers, A.; Kung, D. W.; Zhong, B.; Movassaghi, M.; Kwon, S. J. Am. Chem. Soc. 1999, 121, 8401-8402, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “acyl”, as used herein, refers to a carbonyl-containing finctionality, e.g., —C(═O)R′ wherein R is an aliphatic, heteroaliphatic, aryl, heteroaryl, (aliphatic)aryl, (heteroaliphatic)aryl, heteroaliphatic(aryl) or heteroaliphatic(heteroaryl) moiety, whereby each of the aliphatic, heteroaliphatic, aryl, or heteroaryl moieties is substituted or unsubstituted, or is a substituted (e.g., hydrogen or alipahtic, heteroaliphatic, aryl, or heteroaryl moieties) oxygen or nitrogen containing functionality (e.g., forming a carboxylic acid, ester, or amide functionality).

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂—cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂— cyclopentyl, n-hexyl, sec-hexyl, cyclohexyl, —CH₂—cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1 -propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —SCN; —CF₃; —CH₂CF₃; —CHCl₂; —CH₂OH; —CH₂CH₂OH; —CH₂NH₂; —CH₂SO₂CH₃; —C(O)R; —CO₂(R′); —CON(R′)₂; —OC(O) R′; —OCO₂R′; —OCON(R′)₂; —N(R′)₂; —S(O)₂R′; —N R′(CO) R′ or —B(OR′)₂, wherein each occurrence of R′ independently includes, but is not limited to, aliphatic, heteroaliphatic, aryl, heteroaryl, alkylaryl, or alkylheteroaryl, or wherein any two of R′, taken together is a cyclic aliphatic, heteroaliphatic, aryl or heteroaryl moiety, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —SCN; —CF₃; —CH₂CF₃; —CHCl₂; —CH₂OH; —CH₂CH₂OH; —CH₂NH₂; —CH₂SO₂CH₃; —C(O) R′; —CO₂(R′); —CON(R′)₂; —OC(O) R′; —OCO₂R′; —OCON(R′)₂; —N(R′)₂; —S(O)₂ R′; —N R′(CO) R′ or —B(O R′)₂, wherein each occurrence of R′ independently includes, but is not limited to, aliphatic, heteroaliphatic, aryl, heteroaryl, alkylaryl, or alkylheteroaryl, or wherein any two of R′, taken together is a cyclic aliphatic, heteroaliphatic, aryl or heteroaryl moiety, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —SCN; —CF₃; —CH₂CF₃; —CHCl₂; —CH₂OH; —CH₂CH₂OH; —CH₂NH₂; —CH₂SO₂CH₃; —C(O)R; —CO₂(R′); —CON(R′)₂; —OC(O)R′; —OCO₂ R′; —OCON(R′)₂; —N(R′)₂; —S(O)₂R; —NR (CO)R′ or —B(OR′)₂, wherein each occurrence of R′ independently includes, but is not limited to, aliphatic, heteroaliphatic, aryl, heteroaryl, alkylaryl, or alkylheteroaryl, or wherein any two of R′, taken together is a cyclic aliphatic, heteroaliphatic, aryl or heteroaryl moiety, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties that contain one or more oxygen, sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —SCN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R′; —NR′(CO)R′ or —B(O R′)₂, wherein each occurrence of R′ independently includes, but is not limited to, aliphatic, heteroaliphatic, aryl, heteroaryl, alkylaryl, or alkylheteroaryl, or wherein any two of R′ taken together is a cyclic aliphatic, heteroaliphatic, aryl or heteroaryl moiety, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7- membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —SCN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R; —OCO₂R′; —OCON(R′)₂; —N(R′)₂; —S(O)₂ R′; —NR′(CO)R′ or —B(OR′)₂, wherein each occurrence of R′ independently includes, but is not limited to, aliphatic, heteroaliphatic, aryl, heteroaryl, alkylaryl, or alkylheteroaryl, or wherein any two of R′ taken together is a cyclic aliphatic, heteroaliphatic, aryl or heteroaryl moiety, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “solid support”, as used herein, refers to a material having a rigid or semi-rigid surface. Such materials will preferably take the form of small beads, pellets, disks, chips, dishes, multi-well plates, glass slides, wafers, or the like, although other forms may be used. In some embodiments, at least one surface of the substrate will be substantially flat. The term “surface” refers to any generally two-dimensional structure on a solid substrate and may have steps, ridges, kinks,, terraces, and the like without ceasing to be a surface.

The term “polymeric support”, as used herein, refers to a soluble or insoluble polymer to which an amino acid or other chemical moiety can be covalently bonded by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions.

The term “linker”, as used herein, refers to a chemical moiety utilized to attach a compound of interest to a solid support to facilitate synthesis of inventive compounds. Exemplary linkers are described herein. It will be appreciated that other linkers (including silicon-based linkers and other linkers) that are known in the art can also be employed for the synthesis of the compounds of the invention.

The term “solid support unit” as used herein, refers to a composition comprising a solid support and a linker, as defined above and exemplified herein.

4) Uses, Formulation and Administration

Pharmaceutical Compositions

As discussed above, the present invention provides novel compounds having antitumor and antiproliferative activity, and thus the inventive compounds are useful for the treatment of cancer. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents. In certain other embodiments, the additional therapeutic agent is an anticancer agent, as discussed in more detail herein. In certain other embodiments, compounds of the invention are useful as antiprotozoal agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutical Compositions

As discussed above, the inventive compounds are selective inhibitors of histone deaceytlase and thus are useful in the treatment of cancer. Accordingly, in yet another aspect, according to the methods of treatment of the present invention, tumor cells are killed, or their growth is inhibited by contacting said tumor cells with an inventive compound or composition, as described herein. Thus, in still another aspect of the invention, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells”, as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like. The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar--agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard- filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, the compounds of the present invention are useful as inhibitors of histone deacetylases and thus are useful as anticancer agents, and thus may be useful in the treatment of cancer, by effecting tumor cell death or inhibiting the growth of tumor cells. In general, the inventive anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas.

Additionally, as described above and in the exemplification, the inventive compounds may also be useful in the treatment of protozoal infections. In general, the compounds of the invention are useful for disorders resulting from histone deacetylation activity.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (in but a few examples, y-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and the equivalents thereof.

### EXEMPLIFICATION

### I. EXAMPLE 1

### Synthesis of 1,3-dioxanesfor Use as HDAC Inhibitors

Post-translational modification of proteins through acetylation and deacetylation of lysine residues has a critical role in regulating their cellular finctions (Kouzarides, T. EMBO J 2000, 19, 1176). While small molecule probes for specific protein kinases and phosphatases exist, probes of histone acetyl transferases and deacetylases are limited due to their lack of selectivity. Described below is the synthesis of a library of 1,3-dioxanes and the discovery of certain selective small molecule inhibitors of histone deacetylases (HDACs). Thousands of such compounds using the one bead-one stock solution format ((a) Sternson et al. J. Am. Chem. Soc. 2001, 123, 1740-1747; (b) Blackwell et al. Chem. Biol. 2001, 8, 1167-1182 (c) Clemons et al. Chem. Biol. 2001, 8, 1183-1195) for synthesis have been described below.

HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). HDACs participate in cellular pathways that control cell shape and differentiation, and an HDAC inhibitor has been shown effective in treating an otherwise recalcitrant cancer (Warrell et al. J. Natl. Cancer Inst. 1998, 90, 1621-1625). Nine human HDACs have been characterized ((a) Taunton et al. Science 1996, 272, 408-411; (b) Yang et al. J. Biol. Chem. 1997, 272, 28001-28007. (c) Grozinger et al. Proc. Natl. Acad. Sci U.S.A. 1999, 96, 4868-4873. (d) Kao et al. Genes Dev. 2000, 14, 55-66. (e) Hu et al. J. Biol. Chem. 2000, 275, 15254-15264. (f) Zhou et al. Proc. Natl. Acad. Sci. U.S.A. 2001, 98, 10572-10577) and two inferred (Venter et al. Science 2001, 291, 1304-1351); these members fall into two related classes (class I and II). No small molecules are known that selectively target either the two classes or individual members of this family (for example ortholog-selective HDAC inhibitors have been reported: (a) Meinke et al. J. Med. Chem. 2000, 14, 4919-4922; (b) Meinke, et al. Curr. Med. Chem. 2001, 8, 211-235).

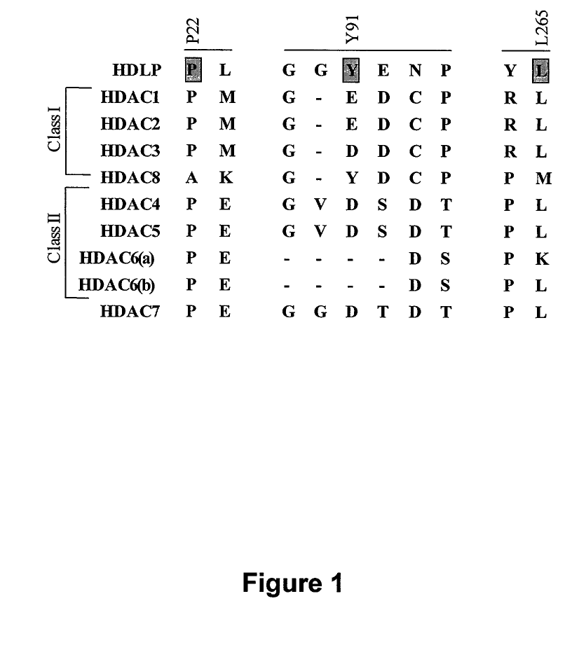

The natural products trapoxin (Kijima et al. J. Biol. Chem. 1993, 268, 22429-22435) (TPX) and trichostatin A (Tsuji et al. J. Antibiot. 1976, 29, 1-6) (TSA) are potent inhibitors and useful probes of HDACs, but their lack of selectivity among family members limits their ability to dissect the functions of individual members. The absence of atomic resolution structures of human HDACs complicates a structure-based solution to this problem; therefore, we have undertaken a screening-based approach. Based on sequence alignments of HDACs and structural analyses of natural HDAC inhibitors (FIG. 1), including TPX and TSA, a synthetic pathway was conceived leading to 7200 1,3-dioxanes, all biased towards HDAC inhibition. Representative compounds that resulted from this pathway were shown to inhibit two different human HDACs; the varied structures of the dioxanes and their tendency to inhibit HDACs suggest that suitable screening methods may identify the sought after, specific inhibitors.

While not wishing to be bound by any particular theory, structural rationale for HDAC inhibition is suggested from the X-ray crystal structure of TSA-bound HDAC-like protein (HDLP), an HDAC ortholog from the thermophilic bacterium Aquifex aeolicus (Finnin et al. Nature 1999, 401, 188-193). In this structure, the hydroxamic acid of TSA penetrates a narrow, hydrophobic channel and chelates a buried zinc ion. The substructural organization of most HDAC inhibitors can be rationalized in light of the HDLP structure. Inhibitors typically possess metal-binding functionality, and a cap substructure that interacts with amino acids at the entrance of the N-acetyl lysine-binding channel. The cap and the metal-binding functionality are connected by a linker, often a 5-6 atom hydrocarbon chain (Jung et al. J. Med. Chem. 1999, 42, 4669-4679). Synthetic molecules incorporating these substructural elements are likely to inhibit HDAC enzymes.

Comparison of amino acid sequences around the active site was used to infer structural differences between individual HDAC family members that could be exploited in the design of selective inhibitors. Most of the amino acids that contact TSA in the HDLP structure are conserved across all HDACs. However, this conservation diverges for amino acids at the solvent-exposed rim of the channel, indicating that this is a selectivity-determining region (FIG. 1). The most significant sequence differences are observed between class I and class II HDACs. The sequence diversity in the rim of the N-acetyl lysine-binding channel suggests that selective inhibitors may be identified from collections of compounds having varied cap groups, since these groups would be expected to interact with the rim residues. It is also of note that an Arg265Pro single nucleotide polymorphism has been recently identified in the rim region of HDAC3, (Wolfsberg et al. Nature 2001, 409, 824-826) providing the structural rationale for polymorph-specific design of HDAC inhibitors. By synthesizing molecules that possess diversity elements targeted towards regions predicted to be structurally divergent, discovery of selective inhibitors may be possible.

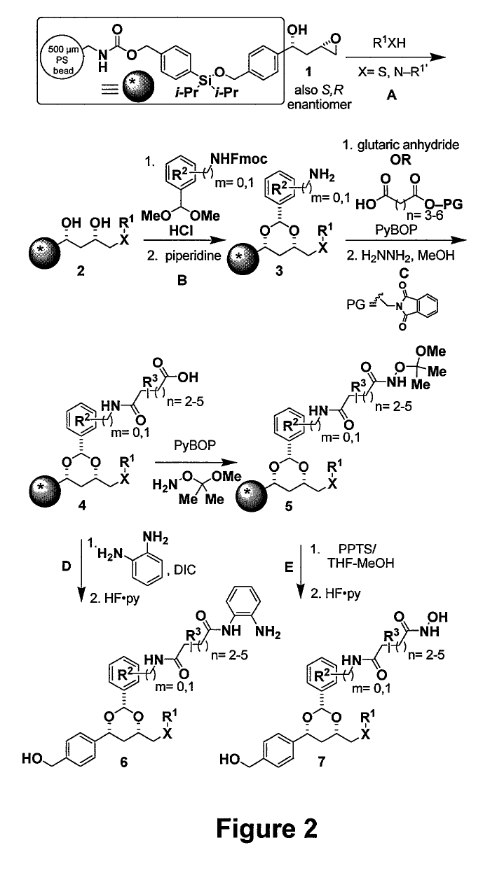

The synthetic plan (FIG. 2) generates diversity in the cap region of the small molecules by using the split-pool synthesis technique. The chain length for the hydrocarbon linker ranges from 3-6 methylene groups so that the orientation of the cap relative to the enzyme channel is varied. The 1,3-dioxane is a rigid core that can be synthesized stereoselectively and with enormous structural diversity (Stemson et al. J. Am. Chem. Soc. 2001, 123, 1740-1747).

To simplify structure determination of synthetic products, an adaptation of the encoding strategy reported by Still and co-workers (Ohlmeyer et al. Proc. Natl Acad. Sci. U.S.A. 1993, 90, 10922-10926) was used entailing covalent [Rh(OCOCPh₃)₂]₂-mediated attachment of electrophoric diazoketone tags to individual, high capacity polystyrene synthesis beads. These tags can be removed oxidatively after the synthesis and analyzed by GC.

Two portions of a silane-derivatized polystyrene resin were tagged with two diazoketones, and, then, enantiomeric γδ-epoxy alcohols attached to afford modified polystyrene support (1). The resin was pooled, split, encoded for the subsequent reactions with 50 combinations of six diazoketones, and reacted with 50 nucleophile building blocks to generate 100 1,3-diols (2) in high purity. The solid-supported 1,3-diols were pooled and split into six portions that were reacted with Fmoc-amino dimethylacetal building blocks under HCl catalysis to form 600 Fmoc-amino-1,3-dioxanes (3). The resin was tagged with six combinations of three diazoketones to encode the ketalization reactions. To encode the subsequent reactions, the resin was pooled and split into four portions and reacted with four combinations of three diazoketones. After Fmoc removal, these pools were reacted with TESC1 to protect free hydroxyls incorporated from the nucleophile building blocks. The amino-1,3-dioxane resin was reacted with four diacid building blocks: pyridine-activated glutaric anhydride or PyBOP-activated monophthalimido-methylester diacids (Nefkens et al. Recueil 1963, 82, 941-953). Treatment with hydrazine generated 2400 carboxyamides (4). One third of these carboxyamide beads was set aside for screening experiments. The high purity of these reaction products indicates that the phthalimidomethylester is well-suited for carboxylic acid protection in solid phase organic synthesis where ester hydrolysis can be difficult due to the poor aqueous swelling properties of polystyrene resins. The remaining carboxyamides were split into two portions. One portion was reacted with diisopropylcarbodiimide (DIC) and phenylenediamine to generate 2400 o-aminoanilides (6). Reaction of the remaining portion of resin 4 with O-2-methoxypropanehydroxylamine in the presence of PyBOP generated 2400 protected hydroxamic acids (5). The 2-methoxypropane protecting group was essential for this reaction as O-TBDMS protection gave impure reaction products and O-allyl and O-THP protecting groups were not sufficiently labile to be removed under conditions compatible with every synthesized compound. Treatment of resin 5 with PPTS generated 2400 hydroxamic acids (7). The purity of the reaction products at each synthetic step was determined by LC-MS analysis of the crude material cleaved from single beads. For every reaction product analyzed (50 out of 50), GC analysis of the electrophoric tags allowed their structures to be inferred. In each instance, the mass of the structure inferred was consistent with the LC-MS data.

The 7200 polystyrene beads were arrayed and cleaved in the one bead-one stock solution format to generate 7200 stock solutions. To demonstrate the ability of the synthesized compounds to inhibit HDAC, we synthesized two compounds (8 and 9) representative of the molecules in the library, and measured IC50s against HDAC1 and HDAC6. Calculated IC50s of ˜1 μM were similar to the IC50s of the substructure 10, indicating that the 1,3-dioxane portion of the molecules is not detrimental to HDAC inhibition. In contrast, a compound with a shorter hydrocarbon linker (11) than those used in the library synthesis weakly inhibited HDAC1 and HDAC6 (IC50s>50 μM).

### II. EXPERIMENTALS FOR EXAMPLE I

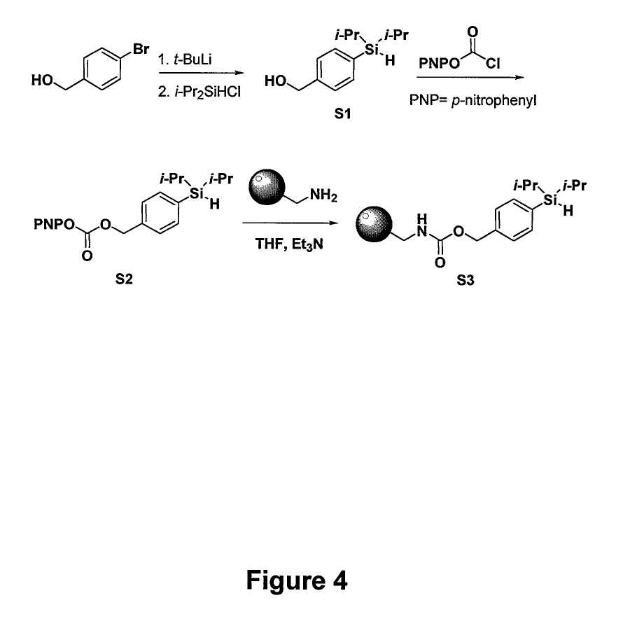

### A) Silyl linker synthesis (FIG. 4)

4-(Diisopropylsilanyl)-phenyl methanol (S1). To a stirred suspension of sodium hydride (2.60 g, 108 mmol) in THF (430 mL) was added a THF (50 ML) solution of 4-bromobenzyl alcohol (18.2 g, 97.3 mmol). The mixture was heated to 45° C. for 3 h and, then, cooled to −65° C. A 1.80 M solution of tert-butyllithium (114 mL) was added to the reaction. After 30 minutes, chlorodiisopropylsilane (17.6 mL, 102 mmol) was added, and the reaction was warmed to room temperature, stirred overnight, and quenched with saturated (NH4) 2SO₄ (15 mL). The resulting mixture was filtered through celite and Na₂SO₄, concentrated in vacuo, and then dissolved in hexanes (400 mL). This solution was washed with water (400 mL), brine (250 mL), then dried over Na₂SO₄, filtered, and concentrated in vacuo to obtain a yellow oil (23 g). Flash column chromatography (silica gel, 20% ethyl acetate/hexanes) provided S1 (17.5 g, 81%) as a colorless oil. ¹H NMR (400 MHz, CDCl₃): δ7.52 (d, 2H, J=1.8 Hz), 7.51 (d, 2H, J=1.8 Hz), 4.70 (d, 2H, J=6.1 Hz), 3.94 (t, 1H, J=3.1Hz), 1.64 (t, 1H, J=6.1 Hz), 1.23 (m, 2H), 1.06 (d, 6H, J=7.3 Hz), 0.98 (d, 6H, J=7.3 Hz). CI/MS (NH₃): 240 (M+NH₄⁺).

Carbonic acid 4-(diisopropyl-silanyl)-benzyl ester 4-nitrophenyl ester (S2). To a solution of silane S2 (0.60 g, 2.7 mmol) in CH₂Cl₂ (10 mL) was added 2,6-lutidine (0.040 mL, 3.5 mmol) followed by 4-nitrophenyl chloroformate (0.60 g, 3 mmol). After 45 minutes, the reaction was concentrated in vacuo. Flash column chromatography (silica gel, 20% ether/hexanes) furnished carbonate (S2) (1.05 g, 100%) as a pale yellow oil that solidified on standing. ¹H NMR (400 MHz, CDCl₃): δ8.28 (d, 2H, J=9.5 Hz), 7.56 (d, 2H, J=8.1 Hz), 7.42 (d, 2H, J=8.1 Hz), 7.40 (d, 2H, J=9.5 Hz), 5.30 (s, 1H), 3.96 (t, 1H, J=3.1 Hz), 1.23 (m, 2H), 1.07 (d, 6H, J=7.3 Hz), 0.99 (d, 6H, J=7.3 Hz). FAB/MS: 410 (M+Na⁺).

Diisopropylphenylsilane resin (S3). PS AM NH₂ resin (1.5 g, 1.41 mmol, 1.0 equiv.) was placed in a 20 mL fritted polypropylene tube. A solution of carbonate, S2, (1.7 g, 4.4 mmol, 3.1 equiv.) in THF (12 mL) with Et₃N (0.558 niL, 4.0 mmol, 4.0 equiv.) was added. The reaction was allowed to proceed for 48 h with rotary mixing. The yellow resin was filtered and washed extensively with H₂O and THF until the resin was white to yield diisopropylphenylsilane resin (S3).

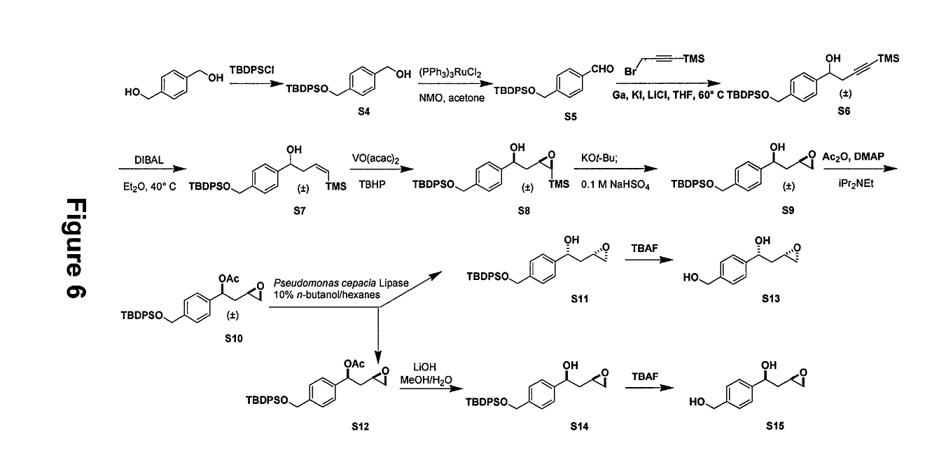

γ,δ-Epoxy alcohol synthesis (FIG. 6)

4-(tert-Butyldiphenylsilanoxymethyl)phenyl methanol (S4). To 1,4-benzenedimethanol (25 g, 180 mmol) in DMF (450 mL) with imidazole (9.2 g, 140 mmol) was added tert-butyldiphenylsilyl chloride (12 mL, 45 mmol). After 24 h the solvent was removed in vacuo and the residue was diluted with water (300 mL). The mixture was extracted with CH₂Cl₂ (2×150 mL), and the organic layers were dried over Na₂SO₄. Flash column chromatography (silica gel, 30% ethyl acetate/hexanes) provided alcohol S4 (14.8 g, 87%) as a colorless oil. ¹H NMR (400 MHz, CDCl₃): 6 (dd, 4H, J=8.0 Hz, 1.5 Hz), 7.35 (m, 10H), 4.77 (s, 2H), 4.70 (s, 2H), 1.10 (s, 9H).

4-(tert-Butyldiphenylsilanoxymethyl)benzaldehyde (S5). To a solution of alcohol S4 (14.5 g, 39.4 mmol) in acetone (200 mL) with N-methylmorpholine oxide (6.9 g, 59 mmol) was added, portionwise, RuCl₂(PPh₃)₃ (0.76 g, 0.79 mmol). After 25 min, the solvent was removed in vacuo. Flash column chromatography (silica gel, 30% ether/hexanes) furnished a white, waxy solid (10.9 g, 74%). ¹H NMR (400 MHz, CDCl₃): δ10.01 (s, 1H), 7.85 (d, 2H, J=8.3 Hz), 7.68 (d, 4H, J=6.3 Hz), 7.51 (d, 2H, J=8.3 Hz), 7.38 (m, 6H), 4.84 (s, 2H), 1.11 (s, 9H).

(±)-1-[4-(tert-Butyldiphenylsilanoxymethyl)phenyl]-4-(trimethylsilanyl)-but-3-yn-1-ol (S6). A flame dried, three-necked flask was charged with aldehyde S5 (9.0 g, 24 mmol) in THF (150 mL), LiCl (1.2 g, 29 mmol), KI (8.0 g, 48 mmol), gallium pellets (2.5 g, 36 mmol), and 3-(trimethylsilyl)propargyl bromide (6.8 mL, 48 mmol). After refluxing for 14 h, the reaction was diluted with ether (300 mL). The resulting mixture was extracted with water (2 ×100 mL), and the aqueous extracts were back extracted with ether (4×50 mL). The organic extracts were dried over MgSO₄ and purified by flash column chromatography (silica gel, 20% ether/hexanes) to generate alkyne S6 as a yellow oil (7.9 g, 68%; 86% based on recovered starting material). ¹H NMR (500 MHz, CDCl₃): δ7.69 (d, 4H, J=7.8 Hz), 7.40 (m, 10H), 4.86 (m, 1H), 4.76 (s, 2H), 2.66 (d, 1H, J=7.1 Hz), 2.41 (d, 1H, J=3.3 Hz), 1.09 (s, 9H), 0.16 (s, 9H).

(±)-1-[4-(tert-Butyldiphenylsilanoxymethyl)phenyl]-4-(trimethylsilanyl)-but-3-en-1-ol (S7). A flame dried, three-necked flask charged with alkyne S6 (16.8 g, 34.5 mmol) in ether (20 mL) was cooled to 0° C. DIBAL (12.5 mL, 70 mmol) was added dropwise over 30 minutes with the reaction temperature kept below 20° C. The solution was heated to 38° C. After 3 h, the reaction was cooled to O C and MeOH (1 mL) was added dropwise followed by Na₂SO₄.10H₂O (50 g, CAUTION- EXOTHERM) and celite (20 g). The reaction was filtered, and the solvent was removed in vacuo. Flash column chromatography (silica gel, 20% ether/hexanes) provided olefin S7 (10.9 g, 65%) as a colorless oil. ¹H NMR (400 MHz, CDC1₃): δ7.70 (dd, 4H, J=8.1 Hz, 1.5 Hz), 7.40 (m, 10H), 4.86 (m, 1H), 4.76 (s, 2H), 2.66 (d, 1H, J=7.1 Hz), 2.41 (d, 1H, J=3.3 Hz), 1.09 (s, 9H), 0.16 (s, 9H). FAB/MS: 511 (M+Na⁺).

(±)-1-[4-(tert-Butyldiphenylsilanoxymethyl)phenyl]-2-[3-(trimethylsilanyl)-oxiranyl ]-ethanol (S8). To a solution of azeotropically dried (2×20 mL toluene) alcohol S7 (15.5 g, 31.7 mmol) in CH₂Cl₂ (250 mL) at 0° C. was added vanadyl acetylacetonate (0.27 g, 1.0 mmol) and tert-butyl hydroperoxide (9.3 mL of a 5.4 M solution in dichloroethane, 50 mmol). After 24 h, Me₂S (5 mL) was added to quench the reaction. The mixture was concentrated and immediately purified by flash column chromatography (silica gel, 25% to 50% ether/hexanes gradient). Epoxide S8 was obtained as a colorless oil (8.0 g, 50%). ¹H NMR (500 MHz, CDCl₃): δ7.69 (m, 2H), 7.44-7.33 (m, 12H), 4.98 (ddd, 1H, J=8.9 Hz, 4.9 Hz, 2.5 Hz), 4.77 (s, 2H), 3.26 (ddd, 1H, J=8.9 Hz, 5.2 Hz, 3.5 Hz), 2.60 (d, 1H, J=2.5 Hz), 2.24 (d, 1H, J=5.2 Hz), 2.11 (ddd, 1H, J=14.4 Hz, 4.5 Hz, 3.5 Hz), 1.78 (ddd, 1H, J=14.4 Hz, 8.9 Hz, 8.9 Hz), 1.09 (s, 9H), 0.15 (s, 9H). CI/MS (NH₃): 522 (M +NH₄⁺).

(±)-1-[4-(tert-Butyldiphenysilanoxymethyl)phenyl]-2-oxiranyl-ethanol (S9). To a solution of epoxide S8 (0.53 g, 1.1 mmol) in THF (10.5 mL) at 0° C. was added potassium tert-butoxide (1.3 mL of a 1.0 M solution in THF, 1.3 mmol). After 18 minutes, 0.1 M NaHSO₄ (27 mL) was added, and the reaction was warmed to room temperature. After 4 h, the reaction was quenched with NaHCO₃ (0.6 g), concentrated in vacuo, and then extracted with ether (4×40 mL). The combined organic layers were dried over Na₂SO₄, filtered, and concentrated in vacuo to obtain an orange oil (0.42 g). This was purified by flash column chromatography (silica gel, 35% ethyl acetate/hexanes) to obtain epoxide S9 as a pale yellow oil (0.30 g, 67%). ¹H NMR (500 MHz, CDCl₃): δ7.69 (d, 2H, J=6.8 Hz), 7.44-7.33 (m, 12H), 4.85 (t, 1H, J=6.1 Hz), 4.77 (s, 2H), 3.03 (m, 1H), 2.77 (dd, 1H, J=4.9 Hz, 4.9 Hz), 2.52 (dd, 1H, J=4.9 Hz, 3.0 Hz), 2.33 (s, 1H), 2.07 (ddd, 1H, J=14.5 Hz, 5.0 Hz, 5.0 Hz), 1.90 (ddd, 1H, J=14.5 Hz, 7.5 Hz, 7.5 Hz), 1.09 (s, 9H). CI/MS (NH₃): 450 (M +NH₄⁺).

(±)-Acetic acid 1-[4-(tert-butyldiphenylsilanoxymethyl)phenyl]-2-oxiranyl-ethyl ester (S10). To a solution of alcohol S9 (779 mg, 1.80 mmol) in CH₂Cl₂ (18 mL) at 0° C. was added acetic anhydride (0.28 mL, 3.0 mmol) and N,N-diisopropylethylamine (0.52 mL, 3.0 mmol) in CH₂Cl₂ (4.4 mL), followed by 4-(dimethylamino)pyridine (0.044 g, 0.36 mmol). After 15 minutes, the solution was warmed to room temperature. The reaction was diluted with CH₂Cl₂ (25 mL) and washed with water (25 mL), 1 N citric acid (25 mL), and brine (25 mL). The combined aqueous layers were extracted with CH₂Cl₂ (3×25 mL) and then the combined organic layers were washed with brine (20 mL), dried over Na₂SO₄, filtered, and concentrated in vacuo. The crude oil was purified by flash column chromatography (silica gel, 20% ethyl acetate/hexanes) to obtain ester S10 as a colorless oil (774 mg, 1.63 mmol, 91%). ¹H NMR (400 MHz, CDCl₃): δ7.69 (m, 2H), 7.44-7.34 (m, 12H), 5.96 (t, 1H, J=7.0 Hz), 4.76 (s, 2H), 2.88 (m, 1H), 2.70 (dd, 1H, J=4.8 Hz, 4.8 Hz), 2.41 (dd, 1H, J=4.8 Hz, 2.2 Hz), 2.14 (ddd, 1H, J=14.0 Hz, 7.0 Hz, 7.0 Hz), 2.10 (s, 3H), 2.01 (ddd, 1H, J=14.0 Hz, 7.0 Hz, 5.0 Hz), 1.09 (s, 9H). FAB/MS: 497 (M+Na⁺).

Enzymatic kinetic resolution of S10. To a stirred solution of Pseudomonas cepacia Lipase (0.88 g, Altus Biologics Catalyst #20 ChiroCLEC™-PC) in 10% n-butanol/hexanes (88 mL) was added a solution of ester S10 (0.76 g, 1.60 mmol) in acetone (4.5 mL). The kinetic resolution was followed by chiral HPLC (10% ethanol/hexanes, 1 mL/min. R,R-whelk-01 column). After 25 h, additional catalyst was added (0.13 g). After 35 h, the reaction was filtered through a silica gel plug (200 g) with 65% ether/hexanes. The filtrate was concentrated in vacuo and purified by flash column chromatography (65% ether/hexanes) to afford (1S)-acetic acid 1-[4-(tert-butyldiphenylsilanyloxymethyl)phenyl]-2-oxiranyl-ethyl ester (S12) (0.39 g, 102% yield, 94% ee) and (1R)-1-[4-(tert-butyldiphenylsilanyloxymethyl)phenyl]-2-oxiranyl-ethanol (0.31 g, 90% yield, 100% ee) (S11). Mosher ester analysis for the determination of absolute configuration is presented below. Enantiomeric excess was determined by chiral HPLC (Table S1).

TABLE S1
Distribution over time of enantiomeric starting materials ((S)-ROAc) and
(R)-ROAc) and single product ((S)-ROH) reported as percent total
integrated peak area.

| | (S)-ROAc | (S)-ROH | (R)-ROAc |
|---|---|---|---|
| Time | (Rₜ = 7.72 min.) | (Rₜ = 8.29 min.) | (Rₜ = 12.10 min.) |
| 20 h | 50% | 45% | 5% |
| 26 h | 50% | 47% | 3% |
| 35 h | 50% | 49% | 1% |

(1S)-1-[4-(tert-Butyldiphenylsilanyloxymethyl)phenyl]-2-oxiranyl-ethanol (S14). To a vigrously stirred methanol (21 mL) solution of ester (S12) (0.36 g, 0.76 mmol) was added LiOH.H₂O (0.072 g, 1.70 mmol) in water (7 mL). After 1 h, the reaction was diluted with ether (50 mL) and washed with brine (50 mL). The aqueous layer was extracted with ether (3×50 mL) and the combined organics were washed with brine (50 mL), dried over sodium sulfate, filtered, and concentrated in vacuo. The residue was purified by flash column chromatography (silica gel, 65% ether/hexanes) to obtain alcohol S14 as a colorless oil (0.31 g, 93%). ¹H NMR (500 MHz, CDCl₃): δ7.69 (d, 2H, J=6.8 Hz), 7.44-7.33 (m, 12H), 4.85 (t, 1H, J=6.1 Hz), 4.77 (s, 2H), 3.03 (m, 1H), 2.77 (dd, 1H, J=4.9 Hz, 4.9 Hz), 2.52 (dd, 1H, J=4.9 Hz, 3.0 Hz), 2.33 (s, 1H), 2.07 (ddd, 1H, J=14.5 Hz, 5.0 Hz, 5.0 Hz), 1.90 (ddd, 1H, J=14.5 Hz, 7.5 Hz, 7.5 Hz), 1.09 (s, 9H). CI/MS (NH₃): 450 (M+NH₄⁺).

(1S)-1-(4-Hydroxymethylphenyl)-2-oxiranyl-ethanol (S15). To a solution of silyl alcohol (S14) (0.32 g, 0.74 mmol) in THF (1.14 mL) was added tetrabutylammonium fluoride hydrate (0.29 g, 1.10 mmol). After 2 h, another portion of tetrabutylammonium fluoride hydrate (0.048 mg, 0.18 mmol) was added. After 3 h, the reaction was concentrated with an N₂ stream and purified by flash column chromatography (silica gel, 3% isopropyl alcohol/ethyl acetate) to obtain alcohol SI5 as a colorless oil (0.11 g, 77%). ¹H NMR (400 MHz, CDCl₃): δ7.40-7.35 (m, 4H), 4.98 (t, 1H, J=Hz), 4.69 (d, 2H, J=3.7 Hz), 3.02 (m, 1H), 2.76 (dd, 1H, J=4.8 Hz, 4.0 Hz), 2.51 (dd, 1H, J=4.8 Hz, 2.6 Hz), 2.44 (d, 1H, J=2.2 Hz, OH), 2.06 (ddd, 1H, J=14.0 Hz, 4.8 Hz, 4.0 Hz), 1.86 (ddd, 1H, J=14.0 Hz, 8.0 Hz, 8.0 Hz), 1.70 (broad s, 1H). CI-HRMS (NH₃) m/z calcd for C₁₁H₁₈NO₃ 212.1287, found 212.1285.

(1R)-1-(4-Hydroxymethylphenyl)-2-oxiranyl-ethanol (S13). ¹H NMR (400 MHz, CDCl₃): δ7.40-7.35 (m, 4H), 4.98 (t, 1H, J=Hz), 4.69 (d, 2H, J=3.7 Hz), 3.02 (m, 1H), 2.76 (dd, 1H, J=4.8 Hz, 4.0 Hz), 2.51 (dd, 1H, J=4.8 Hz, 2.6 Hz), 2.44 (d, 1H, J=2.2 Hz, OH), 2.06 (ddd, 1H, J=14.0 Hz, 4.8 Hz, 4.0 Hz), 1.86 (ddd, 1H, J=14.0 Hz, 8.0 Hz, 8.0 Hz), 1.70 (broad s, 1H). CI-HRMS (NH₃) m/z calcd for C₁₁H₁₈NO₃ 212.1287, found 212.1285.

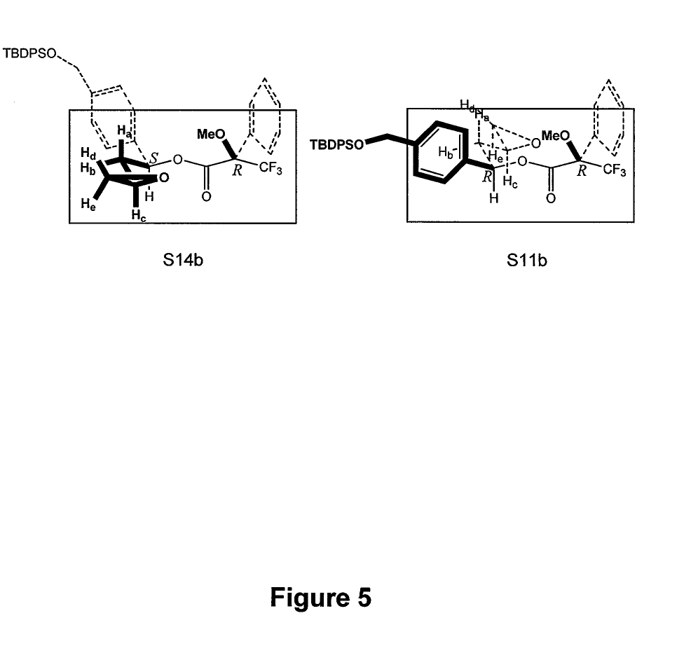

Mosher Ester Derivatization for Determination of Absolute Configuration (FIG. 5)

(R)-(+)-MTPA Ester of (1R)-1-[4-(tert-Butyl-diphenyl-silanyloxymethyl)-phenyl ]-2-oxiranyl-ethanol (S11b). To a stirred CH₂Cl₂ (0.1 mL) solution of (R)-(+)-α-methoxy-α-(trifluoromethyl)phenylacetic acid (3.3 mg, 0.014 mmol), 1-{3-(dimethylamino)propyl]-3-ethylcarbodiimide hydrochloride (2.6 mg, 0.014 mmol), and (dimethylamino)pyridine (0.5 mg, 0.004 mmol), was added a CH₂Cl₂ (0.15 mL) solution of alcohol (S11) (5.0 mg, 0.012 mmol). After 6 h, the reaction was concentrated under an N₂ stream and immediately purified by flash column chromatography (silica gel, 20% ethyl acetate/hexanes) to obtain Mosher ester S11b as a colorless oil (2.8 mg, 0.0043 mmol, 31%). ¹H NMR (400 MHz, CDCl₃): δ7.68 (m, 2H), 7.44-7.31 (m, 19H), 6.13 (dd, 1H, J=6.8 Hz, 6.8 Hz), 4.77 (s, 2H), 3.46 (s, 3H), 2.74 (m, 1H, H_{c}), 2.65 (dd, 1H, J=4.4 Hz, 4.4 Hz, Hd), 2.38 (dd, 1H, J=4.4 Hz, 2.4 Hz, He), 2.26 (ddd, 1H, J=13.6 Hz, 6.8 Hz, 6.8 Hz, Ha), 1.98 (ddd, 1H, J=13.6 Hz, 6.8 Hz, 5.4 Hz, Hb), 1.10 (s, 9H). FAB/MS: 671 (M+Na⁺).

(R)-(+)-MTPA Ester of (1S)-1-[4-(tert-Butyl-diphenyl-silanyloxymethyl)-phenyl ]-2-oxiranyl-ethanol (Sl4b). ¹H NMR (400 MHz, CDCl₃): δ7.69 (m, 2H), 7.45-7.20 (m, 19H), 6.08 (dd, 1H, J=6.8 Hz, 6.8 Hz), 4.76 (s, 2H), 3.56 (s, 3H), 2.84 (m, 1H, H_{c}), 2.71 (dd, 1H, J=4.4 Hz, 4.4 Hz, Hd), 2.44 (dd, 1H, J=4.4 Hz, 2.4 Hz, He), 2.22 (ddd, 1H, J=13.6 Hz, 6.8 Hz, 6.8 Hz, Ha), 2.09 (ddd, 1H, J=13.6 Hz, 6.8 Hz, 4.4 Hz, Hb), 1.10 (s, 9H). FAB/MS: 671 (M+Na⁺).

TABLE S2
Chemical Shift Differences Between Diastereomers S11b and S14b

| Proton | Hₐ | H_{b} | H_{c} | H_{d} | Hₑ |
|---|---|---|---|---|---|
| δS − δR (ppm) | +0.11 | −0.04 | +0.10 | +0.19 | +0.06 |

Table S2 summarizes the observed differences in proton chemical shifts (Δδ) between the (R)-(+)-MPTA esters of the two enantiomers, S14 and S11. Hₐ-Hₑ in S11b are predicted to be upfield of Hₐ-Hₑ in S14b due to the diamagnetic current of the MPTA phenyl ring. The observed Δδ values agree with the predicted Δδ values except in the case of Hb. Although it is difficult to predict the exact orientation of H_{b} relative to the shielding cones of the other phenyl rings in S11b and S14b, the discrepancy between the observed and predicted Δδ's for H_{b} is most likely due to the secondary effect of these rings.

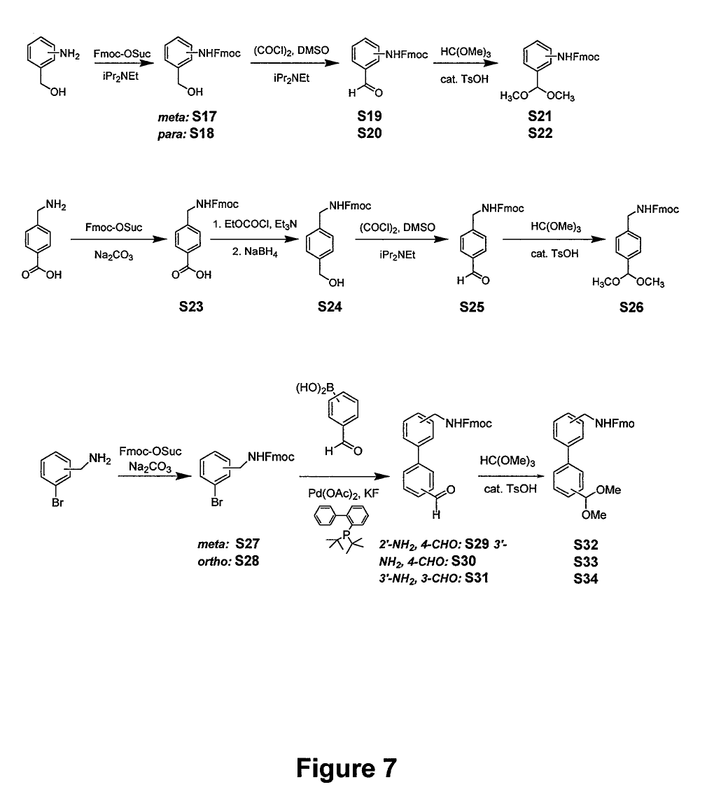

Fmoc-amino dimethyl acetal synthesis (FIG. 7)

(3-Hydroxymethylphenyl)carbamic acid 9H-fluoren-9-ylmethyl ester (S17). To a solution of 3-aminobenzyl alcohol (10 g, 81 mmol) in CH₂Cl₂ (800 mL) and pyridine (8 mL, 100 mmol) was added 9-fluorenylmethyloxycarbonyl-N-hydroxysuccinimide (30 g, 90 mmol) and this heated to 30° C. After 24 h, precipitate had formed. The reaction was filtered and the precipitate was washed with cold CH₂Cl₂ to give alcohol S17 (21.4 g, 76%) as a beige solid. ¹H NMR (500 MHz, CDCl₃): δ7.78 (d, 2H, J=7.8 Hz), 7.61 (d, 2H, J=7.3 Hz), 7.42 (t, 2H, J=7.3 Hz), 7.38 (s, 1H), 7.33 (t, 2H, J=7.3 Hz), 7.31 (m, 2H), 7.05 (m, 1H), 6.75 (br s, 1 H), 4.64 (s, 2H), 4.54 (d, 2H, J=6.4 Hz), 4.27 (t, 1H, J =6.4 Hz). ESI-HRMS m/z calcd for C₂₂H₂₀NO₃ 346.1443, found 346.1427.

(4-Hydroxymethyl-phenyl)-carbamic acid 9H-fluoren-9-ylmethyl ester (S18). To a solution of 4-aminobenzyl alcohol (12.3 g, 100 mmol) in THF (195 mL) and N,N-diisopropylethylamine (17.5 mL, 100 mmol) was added 9-fluorenylmethyloxycarbonyl-N-hydroxysuccinimide (33.8 g, 100 mmol). After 16 h, the reaction was diluted with CH₂Cl₂ and poured into 1 N hydrogen chloride. The aqueous layer was extracted with CH₂Cl₂. The combined organics were dried over magnesium sulfate, filtered, and concentrated to give a yellow solid. This solid was washed with CH₂Cl₂ to give alcohol S18 as a pale yellow solid (23 g, 66%). ¹H NMR (400 MHz, d₆-DMSO): δ9.66 (broad s, 1H), 7.90 (d, 2H, J=7.6 Hz), 7.74 (d, 2H, J=7.3 Hz), 7.42 (dd, 2H, J=7.6 Hz, 7.6 Hz), 7.35 (d, 2H, J=7.7 Hz), 7.34 (dd, 2H, J=7.3 Hz, 7.3 Hz), 7.19 (d, 2H, J=7.7 Hz), 5.07 (t, 1H, J=4.4 Hz), 4.46 (d, 2H, J=6.4 Hz), 4.40 (d, 2H, J=4.4 Hz), 4.29 (t, 1H, J=6.4 Hz). ESI/MS: 368 (M+Na⁺).

(3-Formylphenyl)carbamic acid 9H-fluoren-9-ylmethyl ester (S19). To a stirred, −78° C. CH₂Cl₂ (200 mL) solution of oxalyl chloride (7.8 mL, 89 mmol) was added DMSO (12.7 mL, 178 mmol) in CH₂Cl₂ (40 mL). After 10 minutes, alcohol S17 (23.7 g, 68.7 mmol) in CH₂Cl₂ (65 mL) and DMSO (20 mL) was added over 15 minutes. After 20 minutes, N,N-diisopropylethylamine (60.0 mL, 344 mL) in CH₂Cl₂ (20 mL) was added over 10 minutes and stirred an additional 5 min. The solution was warmed to 0° C. and quenched with water (150 mL). The layers were separated and the aqueous layer was extracted with CH₂Cl₂ (200 mL). The combined organics were washed with brine (150 mL), dried over MgSO₄, filtered, and concentrated in vacuo to obtain a yellow solid. The crude solid was recrystallized (ethyl acetate and hexanes) to obtain a pale yellow solid (11.93 g). The mother liquor was concentrated in vacuo and then purified by flash column chromatography (silica gel, 30% ethyl acetate/hexanes) to obtain a pale yellow solid (6.21 g). Total yield: 18.1 g (77%). ¹H NMR (500 MHz, d₆-DMSO): δ10.1 (broad s, 1H), 9.94 (s, 1H), 8.06 (m, 1H), 7.89 (d, 2H, J=7.3 Hz), 7.75 (d, 2H, J=7.3 Hz), 7.74 (m, 1H), 7.52 (m, 2H), 7.41 (dd, 2H, J=7.3 Hz, 7.3 Hz), 7.34 (dd, 2H, J=7.3 Hz), 4.52 (d, 2H, J=6.4 Hz), 4.32 (t, 1H, J=6.4 Hz). APCI/MS: 344 (M+H⁺)

(4-Formyl-phenyl)-carbamic acid 9H-fluoren-9-ylmethyl ester (S20). ¹H NMR (500 MHz, d₆-DMSO): δ10.20 (broad s, 1H), 9.84 (s, 1H), 7.91 (d, 2H, J=7.8 Hz), 7.81 (d, 2H, J=7.6 Hz), 7.75 (d, 2H, J=7.3 Hz), 7.64 (d, 2H, J=7.6 Hz), 7.42 (dd, 2H, J=7.3 Hz), 7.35 (dd, 2H, J=7.3 Hz), 4.55 (d, 2H, J=6.4 Hz), 4.32 (t, 1H, J=6.4 Hz). APCI/MS: 344 (M+H⁺).

(3-Dimethoxymethylphenyl)carbamic acid 9H-fluoren-9-ylmethyl ester (S21). To a vigorously stirred mixture of methanol (250 mL) containing aldehyde S19 (18 g, 53 mmol) and p-toluenesulfonic acid monohydrate (2.5 g, 13 mmol) was added trimethyl orthoformate (39 mL, 360 mmol). After 1.5 h of moderate heating (flask equipped with short path to drive reaction forward), the reaction was quenched with a 1:1 mixture of saturated sodium bicarbonate and water (150 mL) and diluted with CH₂Cl₂ (200 mL). The aqueous layer was extracted with CH₂Cl₂ and the combined organic layers were washed with brine, dried over Na₂SO₄, and concentrated in vacuo to give dimethyl acetal S21 (20 g, 100%) as a pale yellow solid which was used without further purification. ¹H NMR (500 MHz, d₆-DMSO): δ9.77 (s, 1H), 7.90 (d, 2H, J =7.3 Hz), 7.74 (d, 2H, J=7.3 Hz), 7.55 (br s, 1H), 7.42 (dd, 2H, J=7.3 Hz, 7.3 Hz), 7.41 (m, 1H) 7.34 (dd, 2H, J=7.3 Hz, 7.3 Hz), 7.26 (dd, 1H, J=7.8 Hz, 7.8 Hz), 6.99 (d, 1H, J=7.3), 5.32 (s, 1H), 4.44 (d, 2H, J=6.8 Hz), 4.30 (t, 1H, J=6.8 Hz), 3.21 (s, 6H). APCI/MS: 412 (M+Na⁺).

(4-Dimethoxymethyl-phenyl)-carbamic acid 9H-fluoren-9-ylmethyl ester (S22). ¹H NMR (400 MHz, d₆-DMSO): δ9.75 (broad s, 1H), 7.90 (d, 2H, J=7.3 Hz), 7.74 (d, 2H, J=7.3 Hz), 7.42 (m, 4H), 7.34 (dd, 2H, J=7.3 Hz, 7.3 Hz), 7.25 (d, 2H, J=8.4 Hz), 5.30 (s, 1H), 4.47 (d, 2H, J=6.6 Hz), 4.30 (t, 1H, J=6.6 Hz), 3.20 (s, 6H). ESI/MS: 412 (M+Na⁺).

4-[(9H-Fluoren-9-ylmethoxycarbonylamino)-methyl]-benzoic acid (S23). To 4-aminomethyl-benzoic acid (10.6 g, 70.1 mmol) in dioxane (130 mL) was added 9% aqueous Na₂CO₃ (150 mL) followed by 9-fluorenylmethyloxycarbonyl-N-hydroxysuccinimide (26 g, 77 mmol). The solution was heated to 40° C. for 12 h and then cooled to room temperature. The reaction was acidified with 1 M HCl (500 mL), and extracted with ether (300 mL) to obtain acid S23 as a fluffy white solid (25.9 g, 69.4 mmol, 99%). ¹H NMR (500 MHz, d₆-DMSO): δ12.85 (broad s, 1H), 7.90 (m, 4H), 7.70 (m, 2H), 7.39 (m, 2H), 7.31 (m, 4H), 4.37 (d, 2H, J=6.8 Hz), 4.24 (d, 2H, J=5.8 Hz), 4.23 (t, 1H, J=6.8 Hz). APCI/MS: 372 (M+H⁺).

(4-Hydroxymethylbenzyl)-carbamic acid 9H-fluoren-9-ylmethyl ester (S24). To a solution of acid (S27) (23.3 g, 62.4 mmol) and triethylamine (8.7 mL, 62 mmol) in THF (150 mL) at −7° C. was added ethyl chloroformate (6.0 mL, 62 mmol) over 30 minutes. After an additional 30 minutes, the mixture was filtered through celite and the filter pad was rinsed with THF. NaBH₄ (9.0 g, 240 mmol) was added to the filtrate, followed by dropwise addition of methanol (38 mL) over 1 h at 10° C. After 1.5 h, the reaction was quenched with 3 M HCl and diluted with water and CH₂Cl₂. After 1 h of vigorous stirring, the layers were separated and the aqueous layer extracted with CH₂Cl₂ (2×150 mL). The combined organic layers were washed with brine, dried over MgSO₄, filtered, and concentrated in vacuo to give alcohol (S24) (15.7 g, 70%) which was used without further purification. ¹H NMR (500 MHz, d₆-DMSO): δ7.88 (d, 2H, J=7.3 Hz), 7.82 (t, 1H, J=6.3 Hz, NH), 7.69 (d, 2H, J=7.3 Hz), 7.41 (dd, 2H, J=7.3 Hz, 7.3 Hz), 7.31 (dd, 2H, J=7.3 Hz), 7.23 (d, 2H, J=8.1 Hz), 7.15 (d, 2H, J=8.1 Hz), 5.13 (broad s, 1H), 4.45 (s, 2H), 4.33 (d, 2H, J 6.5 Hz), 4.22 (t, J=6.5 Hz), 4.15 (d, 2H, J=6.3 Hz). APCI/MS: 360 (M+H⁺).

(4-Formylbenzyl)-carbamic acid 9H-fluoren-9-ylmethyl ester (S25). ¹H NMR (500 MHz, d₆-DMSO): 6 10.02 (s, 1H), 7.88 (d, 2H, J=7.6 Hz), 7.87 (d, 2H, J=7.6 Hz), 7.70 (d, 2H, J=7.8), 7.51 (d, 2H, J=7.8), 7.42 (dd, 2H, J=7.6 Hz, 7.6 Hz), 7.33 (dd, 2H, J=7.6 Hz, 7.6 Hz), 7.13 (m, 1H, NH), 4.45 (d, 2H, J=6.4 Hz), 4.42 (d, 2H, J=6.8 Hz), 4.25 (t, 1H, J=6.8 Hz). ESI/MS: 380 (M+Na⁺).

(4-Dimethoxymethylbenzyl)-carbamic acid 9H-fluoren-9-ylmethyl ester (S26). ¹H NMR (500 MHz, d₆-DMSO): δ7.88 (d, 2H, J=7.3 Hz), 7.85 (t, 1H, J=5.9 Hz), 7.68 (d, 2H, J =7.3 Hz), 7.41 (dd, 2H, J=7.3 Hz, 7.3 Hz), 7.31 (m, 4H), 7.20 (d, 2H, J=7.8 Hz), 4.33 (d, 2H, J=6.8 Hz), 4.20 (t, 1H, J=6.8 Hz), 4.17 (d, 2H, J=5.9 Hz), 3.21 (s, 6H). APCI/MS: 426 (M+Na⁺).

(3-Bromobenzyl)-carbamic acid 9H-fluoren-9-ylmethyl ester (S27). To 3-bromobenzylamine hydrochloride (16.0 g, 71.9 mmol) was added aqueous 9% Na₂CO₃ (211 mL) followed by 9-fluorenylmethyloxycarbonyl-N-hydroxysuccinimide (25.5 g, 75.5 mmol) in dioxane (211 mL). This was stirred 30 minutes, then diluted with CH₂Cl₂ (200 mL). The layers were separated and the aqueous layer was extracted with CH₂Cl₂. The combined organic layers were washed with 1 M HCl, brine, dried over magnesium sulfate, filtered, and concentrated in vacuo. The resulting solid was purified by recrystallization (ethyl acetate/hexanes) to obtain a fluffy, white solid (24 g, 59 mmol, 82%). ¹H NMR (400 MHz, d₆-DMSO): 6 7.90 (t, 1H, J=6.2 Hz), 7.88 (d, 2H, J=7.5 Hz), 7.68 (d, 2H, J=7.5 Hz), 7.43 (m, 4H), 7.32 (dd, 2H, J=7.5 Hz, 7.5 Hz), 7.26 (d, 1H, J=7.7 Hz), 7.20 (d, 1H, J=7.3 Hz), 4.35 (d, 2H, J=6.6 Hz), 4.22 (t, 1H, J =6.6 Hz), 4.17 (d, 2H, J=6.2 Hz). ESI/MS: 408 (M+H⁺).

(2-Bromobenzyl)-carbamic acid 9H-fluoren-9-ylmethyl ester (S28). ¹H NMR (400 MHz, d₆-DMSO): δ7.89 (d, 2H, J=7.5 Hz), 7.88 (t, 1H, J=6.2 Hz, NH), 7.71 (d, 2H, J=7.5 Hz, 7.58 (d, 1H, J=7.5 Hz), 7.42 (dd, 2H, J=7.5 Hz, 7.5 Hz), 7.35 (dd, 1H, J=7.5 Hz, 7.5 Hz), 7.32 (dd, 2H, J=7.5 Hz, 7.5 Hz), 7.20 (dd, 1H, J=7.5 Hz, 7.5 Hz), 7.19 (d, 1H, J=7.5 Hz), 4.38 (d, 2H, J=6.6 Hz), 4.24 (t, 1H, J=6.6 Hz), 4.19 (d, 2H, J=6.2 Hz). APCI/MS: 408 (M+H⁺).

(4′-Formylbiphenyl-2-ylmethyl)-carbamic acid 9H-fluoren-9-ylmethyl ester (S29). 4-Formylphenylboronic acid (4.97 g, 33.2 mmol), aryl bromide S28 (9.0 g, 22 mmol), KF (3.8 g, 66 mmol), biphenyl-2-yl-di-tert-butyl-phosphane (0.26 g, 0.884 mmol), and palladium(II) acetate (0.099 g, 0.44 mmol) were charged into an oven-dried flask. After one evacuation/backfill cycle with Ar, the solids were dissolved in THF (45 mL) and heated moderately (30° C.) with stirring. After 24 h, the reaction was diluted with CH₂Cl₂ and was washed with 1 M NaOH. The aqueous layer was extracted with CH₂Cl₂ and the combined organics were then washed with brine, dried over Na₂SO₄, filtered, and concentrated in vacuo. The crude solid was purified by flash column chromatography (silica gel, 30% ethyl acetate/hexanes) to obtain a white solid (6.0 g, 63%). ¹H NMR (400 MHz, d₆-DMSO): δ10.04 (s, 1H), 7.95 (d, 2H, J=8.2 Hz), 7.88 (d, 2H, J=7.5 Hz), 7.82 (t, 1H, J=5.9 Hz, NH), 7.67 (d, 2H, J=7.5 Hz), 7.59 (d, 2H, J=8.2 Hz), 7.43-7.30 (m, 7H), 7.24 (d, 1H, J=7.7 Hz), 4.29 (d, 2H, J=7.0 Hz), 4.19 (t, 1H, J=7.0 Hz), 4.12 (d, 2H, J=5.9 Hz). APCI/MS: 434 (M+H⁺).

(4′-Formylbiphenyl-3-ylmethyl)-carbamic acid 9H-fluoren-9-ylmethyl ester (S30). 4-Formylphenylboronic acid (5.0 g, 33 mmol), aryl bromide S27 (9.0 g, 22 mmol), KF (3.8 g, 66 mmol), biphenyl-2-yl-di-tert-butyl-phosphane (0.26 g, 0.88 mmol), and palladium(II) acetate (0.099 g, 0.44 mmol) were charged into an oven-dried flask. After one evacuation/backfill cycle with Ar, the solids were dissolved in THF (45 mL) and heated to reflux with stirring. After 5 h, the reaction was diluted with ethyl acetate, filtered through a celite pad and washed with 1 M NaOH. The aqueous layer was extracted with ethyl acetate and the combined organics were then washed with brine, dried over MgSO₄, filtered, and concentrated in vacuo to afford an orange oil (13 g). The crude oil was purified by flash column chromatography (silica gel, 30% ethyl acetate/hexanes) to obtain aldehyde S29 as a yellow oil (5.7 g, 60%). ¹H NMR (400 MHz, CDCl₃): δ10.06 (s, 1H), 7.95 (d, 2H, J=8.2 Hz), 7.76 (d, 2H, J=7.5 Hz), 7.74 (d, 2H, J=8.2 Hz), 7.59 (d, 2H, J=7.5 Hz), 7.56 (m, 1H), 7.55 (s, 1H), 7.46 (dd, 1H, J=7.9 Hz, 7.9 Hz), 7.39 (dd, 2H, J=7.5 Hz, 7.5 Hz), 7.33 (m, 1H), 7.29 (dd, 2H, J=7.5 Hz, 7.5 Hz), 5.16 (broad s, 1H), 4.48 (d, 2H, J=7.0 Hz), 4.47 (s, 2H), 4.24 (t, 1H, J=7.0 Hz). ESI/MS: 456 (M+Na⁺).

(3′-Formylbiphenyl-3-ylmethyl)-carbamic acid 9H-fluoren-9-ylmethyl ester (S31). ¹H NMR (400 MHz, CDCl₃): δ10.08 (s, 1H), 8.08 (s, 1H), 7.87 (d, 1H, J=7.7 Hz), 7.84 (d, 1H, J=8.1 Hz), 7.76 (d, 2H, J=7.3 Hz), 7.61 (dd, 1H, J=7.7 Hz, 7.7 Hz), 7.60 (d, 2H, J =7.3 Hz), 7.55 (m, 1H), 7.54 (s, 1H), 7.46 (dd, 1H, J=8.1 Hz, 8.1 Hz), 7.39 (dd, 2H, J=7.3 Hz, 7.3 Hz), 7.32 (m, 1H), 7.39 (dd, 2H, J=7.3 Hz, 7.3 Hz), 5.19 (broad s, 1H) 4.47 (d, 2H, J=7.0 Hz) 4.46 (s, 2H), 4.12 (t, 1H, J=7.0 Hz). APCI/MS: 456 (M+Na⁺).

(4′-Dimethoxymethylbiphenyl-2-ylmethyl)-carbamic acid 9H-fluoren-9-ylmethyl ester (S32). To a vigorously stirred mixture of methanol (250 mL) containing aldehyde (S29) (17.0 g, 39.3 mmol) and p-toluenesulfonic acid monohydrate (0.75 g, 3.9 mmol) was added trimethyl orthoformate (17 mL, 160 mmol). After 1.5 h of moderate heating (flask equipped with short path to drive reaction forward), the reaction was quenched with a 1:1 mixture of saturated sodium bicarbonate and water (150 mL) and diluted with CH₂Cl₂ (200 mL). The aqueous layer was extracted with CH₂Cl₂ and the combined organic layers were washed with brine, dried over Na₂SO₄, and concentrated in vacuo to give a pale yellow solid (17.5 g, 36.6 mmol, 93%) which was used without further purification. ¹H NMR (400 MHz, d₆-DMSO): δ7.88 (d, 2H, J=7.7 Hz), 7.79 (t, 1H, J=5.9 Hz), 7.68 (d, 2H, J=7.3 Hz), 7.43-7.30 (m, 1 1H), 7.19 (d, 1H, J=7.0 Hz), 5.40 (s, 1H), 4.29 (d, 2H, J=7.0 Hz), 4.20 (t, 1H, J=7.0 Hz), 4.11 (d, 2H, J=5.9 Hz), 3.25 (s, 6H). FAB/MS: 502 (M+Na⁺).

(4′-Dimethoxymethylbiphenyl-3-ylmethyl)-carbamic acid 9H-fluoren-9-ylmethyl ester (S33). ¹H NMR (400 MHz, d₆-DMSO): δ7.91 (t, 1H, J=6.1 Hz), 7.87 (d, 2H, J=7.5 Hz), 7.68 (d, 2H, J=7.5 Hz), 7.63 (d, 2H, J=8.4 Hz), 7.55 (s, 1H), 7.53 (m, 1H), 7.45 (d, 1H, J=8.4 Hz), 7.38 (m, 4H), 7.27 (dd, 2H, J=7.5 Hz, 7.5 Hz), 7.21 (d, 1H, J =7.3 Hz), 5.43 (s, 1H), 4.33 (d, 2H, J=7.0 Hz), 4.24 (d, 2H, J=6.1 Hz), 4.23 (t, 1H, J=7.0 Hz), 3.26 (s, 6H). ESI/MS: 502 (M+Na⁺).

(3′-Dimethoxymethyl-biphenyl-3-ylmethyl)-carbamic acid 9H-fluoren-9-ylmethyl ester (S34). ¹H NMR (400 MHz, d₆-DMSO): δ7.94 (t, 1H, J=5.9 Hz), 7.87 (d, 2H, J=7.5 Hz), 7.68 (d, 2H, J=7.5 Hz), 7.60 (m, 1H), 7.59 (s, 1H), 7.52 (m, 2H), 7.47 (dd, 1H, J=7.3 Hz), 7.43-7.37 (m, 4H), 7.27 (dd, 2H, J=7.5 Hz, 7.5 Hz), 7.22 (d, 1H, J=7.3 Hz), 5.42 (s, 1H), 4.33 (d, 2H, J=7.0 Hz), 4.26 (d, 2H, J=5.9 Hz), 4.22 (t, 1H, J=7.0 Hz), 3.25 (s, 6H). ESI/MS: 502 (M+Na⁺).

Methylphthalimidomonoester synthesis

Suberic acid methylphthalimidomonoester (S35). To a mixture of suberic acid (5.0 g, 29 mmol) in DMF (40 mL) with dicyclohexylamine (8.0 mL, 40 mmol) was added chloromethylphthalimide (6.1 g, 31 mmol) and the reaction was heated to 60° C. for 5h. The reaction was diluted with CH₂Cl₂ (500 mL) and washed with 0.1 M NaHSO₄ (3×200 mL). The aqueous layers were back extracted with CH₂Cl₂ (5×100 mL) and the organic layers were combined and dried over MgSO₄, filtered, and concentrated in vacuo. The crude residue was purified by flash column chromatography (silica gel, 7% MeOH/CH₂Cl₂) to yield suberic acid methylphthalimidomonoester as a white solid (3.41 g, 35%). ¹H NMR (500 MHz, CD₃OD): δ7.94 (dd, 2H, J=5.6 Hz, 2.9 Hz), 7.86 (dd, 2H, J=5.6 Hz, 2.9 Hz), 5.68 (s, 2H), 2.32 (t, 2H, J=7.3 Hz), 2.24 (t, 2H, J=7.3 Hz), 1.68 (m, 4H), 1.32 (m, 4H).

Adipic acid methylphthalimidomonoester (S36). ¹H NMR (500 MHz, CDCl₃): δ7.93 (dd, 2H, J=5.4 Hz, 2.9 Hz), 7.79 (dd, 2H, J=5.4 Hz, 2.9 Hz), 5.72 (s, 2H), 2.35 (td, 4H, J =6.8 Hz, 2.0 Hz), 1.67 (m, 4H). ESI-HRMS m/z calcd for C₁₅H₁₅NO₆Na 328.0797; found 328.0801.

Pimelic acid methylphthalimidomonoester (S37). ¹H NMR (500 MHz, CDCl₃): δ7.93 (dd, 2H, J=5.4 Hz, 2.9 Hz), 7.79 (dd, 2H, J=5.4 Hz, 2.9 Hz), 5.72 (s, 2H), 2.33 (m, 4H), 1.64 (m, 4H), 1.36 (m, 2H). ESI-HRMS m/z calcd for C₁₆H₁₇NO₆Na 342.0954; found 342.0967.

Building block selection and demonstration compound synthesis

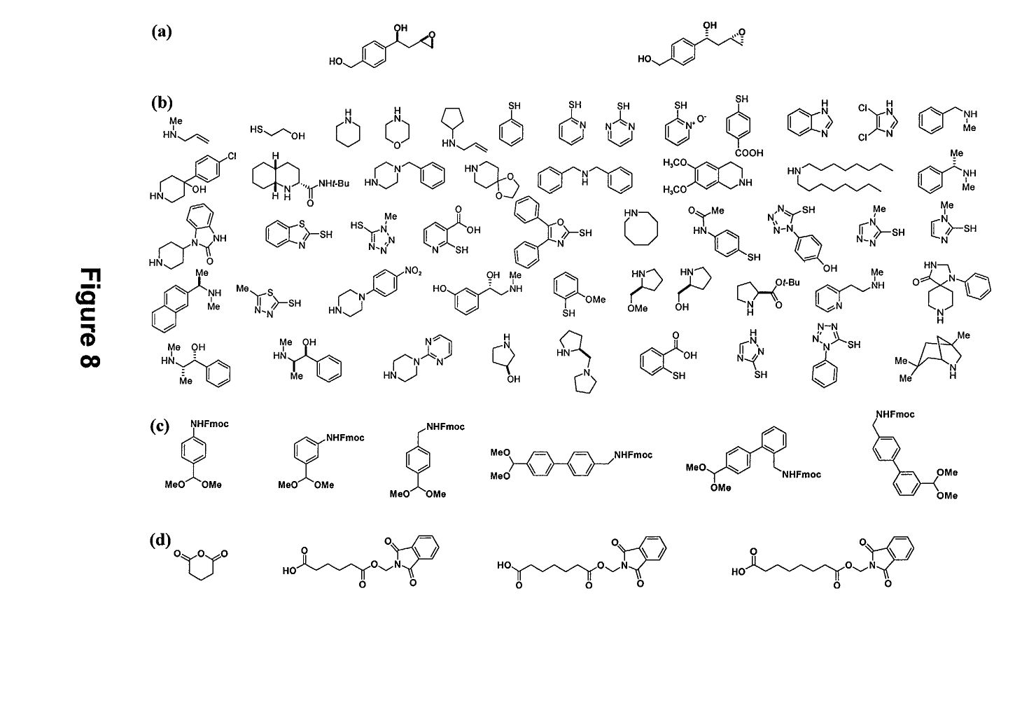

Nucleophile, acetal, and diacid building blocks were selected after analysis of their reactions with model substrates. LC-MS analysis of crude reaction products after HF.pyr cleavage permitted selection of building blocks that underwent reaction with >90% purity (FIG. 8).

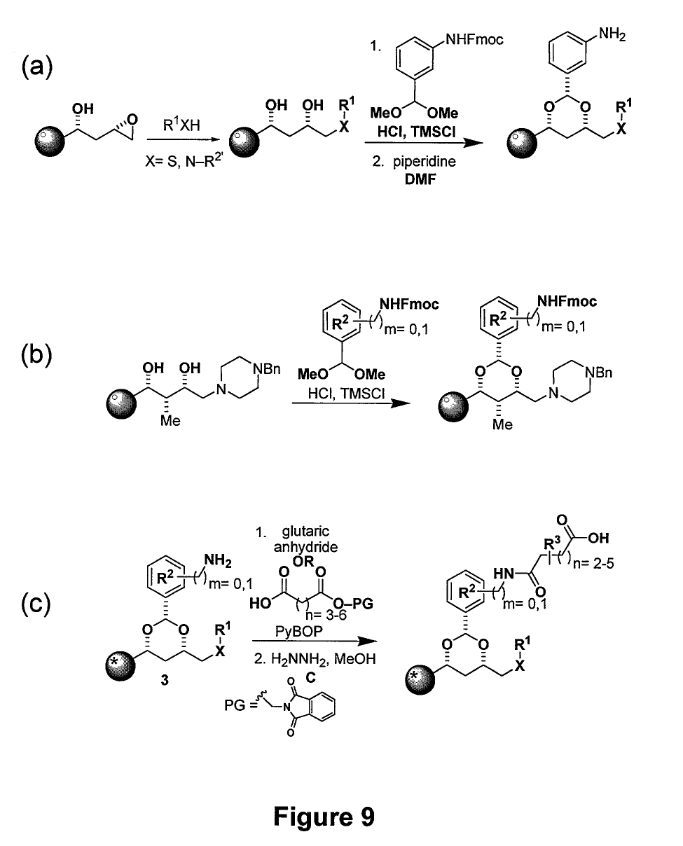

Procedure for selection of nucleophile building blocks. Nucleophilic building blocks were selected after a two step reaction sequence in order to assess the compatibility of different nucleophiles with both the epoxide opening conditions and the acidic ketalization conditions (FIG. 9a).

Procedure for selection of Fmoc-amino dimethylacetal building blocks. Fmoc-amino dimethylacetal building blocks were selected by acetal formation with a 1,3-diol known to be one of the most challenging substrates in the library for this reaction (FIG. 9b).

Procedure for selection of diacid building blocks. Diacid building blocks were selected by reaction with a variety of amino-1,3-dioxanes to ensure appropriate reactivity and purity (FIG. 9c). Several sample hydroxamic acid compounds were also made to demonstrate the purity of the crude reaction products.

(±)-Pentanedioic acid 4-[4-(benzothiazol-2-ylsulfanylmethyl)-6-(4-hydroxymethyl-phenyl)-[1,3]dioxan-2-yl ]-benzylamide hydroxyamide (8). ¹H NMR (500 MHz, CD₃OD): δ7.82 (d, 2H, J=8.1 Hz), 7.46-7.31 (m, 9H), 7.19 (d, 2H, J=7.8 Hz), 5.75 (s, 1H), 4.95 (dd, 1H, J=11.0, 1.8 Hz), 4.58 (s, 2H), 4.41 (m, 1H), 4.33 (s, 2H), 3.69 (dd, 1H, J=13.7 Hz, 4.4 Hz), 3.60 (dd, 1H, J=13.7 Hz, 7.3 Hz), 2.26 (t, 2H, J=7.3 Hz), 2.11 (t, 2H, J=7.3 Hz), 2.07 (m, 1H), 1.92 (quint, 2H, J=7.3 Hz), 1.79 (m, 1H). APCI/MS: 630 (M+Na⁺).

(±)-Octanedioic acid {3-[4-(benzothiazol-2-ylsulfanylmethyl)-6-(4-hydroxymethyl-phenyl)-[1,3]dioxan-2-yl]-phenyl}-amide hydroxyamide (9). ¹H NMR (500 MHz, CD₃OD): δ7.83 (t, 2H, J=9.3 Hz), 7.72 (s, 1H), 7.57 (dt, 1H, J=7.8 Hz, 1.5 Hz), 7.46-7.40 (m, 3H), 7.36-7.32 (m, 3H), 7.22 (t, 1H, J=7.8 Hz), 7.18 (d, 1H, J=7.8 Hz), 5.76 (s, 1H), 4.95 (dd, 1H, J=11.0, 2.2 Hz), 4.59 (s, 2H), 4.43 (m, 1H), 3.71 (dd, 1H, J=13.7 Hz, 4.4 Hz), 3.59 (dd, 1H, J =13.7 Hz, 7.3 Hz), 2.35 (t, 2H, J=7.3 Hz), 2.15-2.05 (m, 3H), 1.79 (m, 1H), 1.69 (t, 2H, J=6.8 Hz), 1.62 (t, 2H, J=7.3 Hz), 1.38 (m, 4H). APCI/MS: 636 (M+H⁺).

Library synthesis and post-synthetic characterization

General procedure for molecular encoding of reactions. To each batch of resin was added a solution in CH₂Cl₂ (3.18 mL) that was 8.4 mM in each tag assigned to that reaction. After 45 min of agitation, a solution of [Rh(OCOCPh₃)₂]₂ in CH₂Cl₂ (3.18 mL of a 4.4 mg/mL solution) was added and the reactions were agitated for 16 h. Reactions were filtered and washed THF (2×15 min) and CH₂Cl₂ (3×1 h). Resin was dried under vacuum.

General procedure for molecular decoding of reactions. To a single bead in a glass HPLC vial insert was added a 0.24 M solution of CAN in 5:1 THF/H₂O (5 μL) followed by decane (8 μL). The inserts were placed in an HPLC vial, capped, and heated at 37° C. for 21 h. The insert was then removed from the vial, sonicated for 1 min and centrifuged briefly. The top decane layer was carefully removed and transferred to a clean glass HPLC insert. A 1:1 solution of N,O-bistrimethylsilylacetamide in decane (1 μL) was added to the decane solution and quickly centrifuged for 30 s. The insert was placed in a GC autosampler vial for EC-GC analysis. EC-GC data was obtained on a Hewlett Packard 6890 gas chromatograph fitted with a 7683 series injector and autosampler, split-splitless inlet, μ-ECD detector, and a J&W DB1 15 m×0.25 mm ×0.25 μm column. (Gradient start temperature: 110° C.; hold 1 min, ramp 45° C./min to 250° C., hold 2 min, ramp 15° C./min to 325° C., hold 2 min. Flow rate: constant flow, 1 mL/min. Inlet was purged at 1 min with flow rate 60 mL/min, reduced to 20 mL/min at 2 min).

Solid phase synthesis procedures

Representative procedure for γ,δ-epoxy alcohol resins (1). Diisopropylphenylsilane resin, S3, (0.76 g, 0.59 mmol, 1.0 equiv.) was split into two portions and encoded for the subsequent y,8-epoxy alcohol building blocks. The resin was placed in an oven-dried 25 mL round bottom flask under Ar. To remove atmospheric water from the resin, it was washed with anhydrous THF (3×10 mL over 30 min) followed by anhydrous CH₂Cl₂ (2 x 10 mL over 20 min). A small-bore cannula was used to remove the solvents. The resin was suspended in CH₂Cl₂ (5 mL) and trichloroisocyanuric acid (0.081 g, 0.35 mmol, 1.2 equiv.) was added. After 1 h, a white precipitate had developed and the resin was filtered via cannula and washed with THF (2×10 mL) followed by CH₂Cl₂ (2×10 mL). γ,δ-Epoxy alcohol (0.10 g, 0.53 mmol, 1.8 equiv.) was azeotropically dried (3×10 mL toluene) and dissolved in CH₂Cl₂ (1 mL) with i-Pr₂NEt (0.092 μL, 0.53 mmol, 1.8 equiv.) and DMAP (0.013 g, 0.11 mmol, 0.4 equiv.). The solution was added to the activated resin and allowed to stand for 4 h. The resin was washed 3×DMF and 5×THF to give γ,δ-epoxy alcohol resin (1).

1,3-diol resin (2). γ,δ-Epoxy alcohol resin, 1, were pooled, suspended in DMF (15 mL), and mixed on a rotary shaker for 1.5 h followed by mixing in THF (15 mL) for 2 h. The resin was filtered and dried under vacuum. The dried resin (19 mg, 0.6 mequiv./g avg, 0.011 mmol, 1.0 equiv.) was split into fifty portions and encoded for the subsequent reaction. To each of the fifty resin portions was added the appropriate nucleophile (0.3 mmol, 27 equiv.) followed by i-PrOH (0.3 mL). In the case of thiol building blocks or amine hydrochloride salts, one equivalent of i-Pr₂NEt (0.051 mL, 0.30 mmol, 27 equiv.) was added and the vials were flushed with Ar, capped, and allowed to stand in an oven at 50° C. 24 h. The reactions were filtered and washed with DMF (10×5 min) and THF (10×5 min) to give 1,3-diol resin (2). Twelve beads were individually cleaved with 19:1 THF/HF.pyr. The tagged beads were decoded, and the crude reaction mixtures were analyzed by LC (FIG. S7).

1,3-Dioxane resin (3). After pooling, 1,3-diol resin, 2, was split into six equal portions (0.19 g, 0.55 mequiv./g avg, 0.10 mmol, 1.0 equiv.) and treated with Fmoc-amino dimethylacetal building blocks (1.1 mmol., 11 equiv.) in a solution of 0.05 M HCl in anhydrous 1,4-dioxane (3.5 mL) and TMSCl (0.35 mL, 2.8 mmol, 28 equiv.). After 4 h, the reaction was quenched with anhydrous pyridine (2 mL), filtered, and washed with DMF (4×10 min) and THF (4×10 min). The resin was treated with 0.2 M pyridiniumpara-toluenesulfonate in 10% MeOH-THF (2×5 mL) for 2 h. The resin was filtered and washed with DMF (4 x 10 min), THF (2×10 min), and CH₂Cl₂ (2×10 min). The pools were then encoded for the previous reaction. The resin was pooled, split into four portions, and encoded for the following reaction. Ten beads were individually cleaved with 19:1 THF/HF-pyr. The tagged beads were decoded, and the crude reaction mixtures were analyzed by LC-MS (FIG. S8). The encoded resin was treated with 20% piperidine-DMF (3×10 mL) for 30 min and then washed with CH₂Cl₂ (4×10 min) and dried under vacuum. The resin was suspended in CH₂Cl₂ (3 mL) with i-Pr₂NEt (0.26 mL, 1.5 mmol) and DMAP (0.012 g, 0.1 mmol) and TESC1 (0.15 mL, 0.9 mmol) was added. After 2 h, the resin was washed with CH₂Cl₂ (4×10 min) to give 1,3-dioxane resin (3).

Carboxylic acid-1,3-dioxane resin (4).

(i)1,3-Dioxane resin (3, 0.30 g) was suspended in DMF (2 mL) with pyridine (0.18 mL, 2 mmol) and glutaric anhydride was added (0.11 g, 1 mmol). After 10 h, the reaction was filtered and washed with DMF (4×10 min), THF (2×10 min), and CH₂Cl₂ (2×10 min).

(ii) 1,3-Dioxane resin (3, 0.30 g) was added to 2:1 DMF/CH₂Cl₂ (2 mL) with acid (2 mmol), PyBOP (0.99 g, 1.9 mmol), and i-Pr₂NEt (0.44 mL, 2.5 mmol). After 12 h, the reactions were filtered and washed with DMF (4 x 10 min), THF (2×10 min), and CH₂Cl₂ (2×10 min). The resin was suspended in 1 M hydrazine in MeOH (1.5 mL, 1.5 mmol) and the mixture was heated to 55° C. After 12 h, the reactions were filtered and washed with DMF (4×10 min), THF (2×10 min), and CH₂Cl₂ (2×10 min). The resin was pooled and split into three equal portions. One third of the resin was set aside for biological testing. Nine beads were individually cleaved with 19:1 THF/HF.pyr. The tagged beads were decoded, and the crude reaction mixtures were analyzed by LC-MS.

o-Aminoanilide 1,3-dioxane resin (6). One third of the carboxylic acid 1,3-dioxane resin (5, 0.43 g) was combined with 1-hydroxybenzotriazole (0.076 g, 0.56 mmol) and 1,2-phenylenediamine (0.080 g, 0.74 mmol. A solution of i-Pr₂NEt (0.21 mL, 1.2 mmol) in CH₂Cl₂ (2 mL) was added followed by diisopropylcarbodiimide (0.095 mL, 0.60 mmol). After 4 h, the reaction was filtered and washed with DMF (4×10 min), THF (2×10 min), and CH₂Cl₂ (2 x 10 min). Ten beads were individually cleaved with 18:1:1 THF/HF.pyr/pyr. The tagged beads were decoded, and the crude reaction mixtures were analyzed by LC-MS.

Hydroxamic acid 1,3-dioxane resin (7). One third of the carboxylic acid 1,3-dioxane resin (5, 0.43 g) was suspended in DMF (2 mL) with PyBOP (0.3 g, 0.58 mmol), i-Pr₂NEt (0.21 mL, 1.2 mmol), and O-(2-methoxypropane)-hydroxylamine (0.08 g, 0.76 mmol). After 5 h, the reaction was filtered and washed with DMF (4×10 min). The resin was then treated with 0.2 M pyridinium para-toluenesulfonate in 10% MeOH-THF (2×5 mL) for 2 h. The resin was filtered and washed with DMF (4×10 min), THF (2×10 min), CH₂Cl₂ (2×10 min). Ten beads were individually cleaved with 19:1 THF/HF.pyr. The tagged beads were decoded, and the crude reaction mixtures were analyzed by LC-MS.

Post-synthetic characterization

After key steps in the library synthesis single beads were cleaved and analyzed for the purity of the attached compounds and to confirm proper encoding of the bead. The HPLC UV traces of these single bead cleavages are shown. In all cases, the molecular ion in the MS trace of the major peak corresponded to the mass predicted by the encoding reactions. The lower purity for the library synthesis versus the model reactions demonstrates the potential for discrepancy in results between model reactions and products from a library synthesis that occurs at the final steps.

LC-MS analysis was performed on a Micromass Platform LCZ-MS coupled to a Waters 2690 HPLC. Analyses were run using either an APCI or an ES interface with positive-negative ionization mode switching. Chromatography was over a 3.5 μm Waters Symmetry C18 column (50 mm×2.1 mm i.d.) eluting at 0.4 mL/min with a gradient of 15-100% B over 10 min (A=water+0.1% formic acid; B=acetonitrile+0.1% formic acid). A 5 μL sample of the solution was injected.

Library cleavage elution studies

To ensure that our high-throughput process for library cleavage would yield stock solution concentrations sufficient for multiple phenotypic and protein-binding assays, a model study was performed.

Compound S35 was synthesized on 20 milligrams (182 beads) of γ,δ-epoxyol functionalized resin (1). Nineteen beads were set aside for single bead cleavage and elution experiments. The remaining beads were treated with 18:1:1 THF/Hf.pyr/pyr for two hours. The cleavage reaction was quenched with TMSOMe and the resultant solution separated from the beads. The beads were then washed with THF for one day to ensure maximum extraction of S35 from the polymer matrix. Purification of the combined extracts by silica gel chromatography resulted in recovery of 56 nmol/bead.

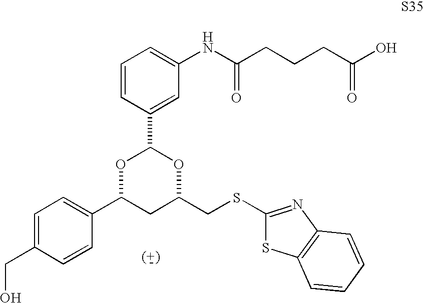

Nineteen uncleaved beads were arrayed into a 384-well late (one bead/well). Ten beads were treated with 18: 1:1 THF/HF.pyr/pyr (20 μL/well) for two hours. The remaining nine beads were treated with this cleavage solution for five hours. The reactions were quenched with TMSOME (20 μL/well) and the solution in each well evaporated over fifteen minutes. Compound S35 was then eluted from each bead with 20 minute washes using 20 μL DMF. A two-wash protocol was determined to be optimal (single bead yield did not increase with additional washes). Eluent was then removed using a centrifugal vacuum evaporator (GeneVac) and the dried material in each well dissolved in 5 μL of DMF. The amount of compound in each well was determined spectrophotometrically by comparison to a standard curve generated from purified S35.

Two hour cleavage was sufficient to obtain single bead yields which were comparable, on average, to the bulk cleavage yield. However, five hour cleavage minimized bead-to-bead deviations, presumably because the longer reaction time enables full reagent penetration into the polymer support.

Compound cleavage and formation of arrayed stock solutions.

Resin was distributed into twenty-one 384-well polypropylene plates (Genetix, 50 μL well volume) using a bead arraying tool to give a single bead per well. Each well was treated with a solution of 18:1:1 THF/HF-pyr/pyr (20 μL). After 2 h, TMSOMe (20 μL) was added to quench the HF. The solvent was allowed to evaporate and the beads were washed with DMF (3 ×15 μL×40 min) and distributed into daughter plates. DMF was removed with a centrifugal vacuum evaporator (GeneVac). Compounds will be dissolved in a polar solvent prior to biological assay.

The 2 h cleavage protocol was selected, despite more consistent stock solution concentrations using a 5 h protocol, because exposing model compounds to 18:1:1 THF/HF.pyr/pyr for longer than 2 h decreased their purity.

Biological Assay Procedures

Cell culture and Transfections. TAg-Jurkat cells were transfected by electroporation with 5 μg of FLAG-epitope-tagged pBJ5 constructs (as described in ref. 5c) for expression of recombinant proteins. Cells were harvested 48 h posttransfection.

HDAC assays. [³H]Acetate-incorporated histones were isolated from butyrate-treated HeLa cells by hydroxyapatite chromatography (as described in Tong, et al. Nature 1997, 395, 917-921.) Immunoprecipitates were incubated with 1.4 μg (10,000 dpm) histones for 3 h at 37° C. HDAC activity was determined by scintillation counting of the ethyl acetate-soluble [³H]acetic acid (as described in Taunton, et al., Science 1996, 272, 408-411). Compounds were added in DMSO such that final assay concentrations were 1% DMSO. IC50s were calculated using Prism 3.0 software. Curve fitting was done without constraints using the program's Sigmoidal-Dose Response parameters. All data points were acquired in duplicate and IC50s are calculated from the composite results of at least two separate experiments.

Identification and Characterization of a Selective Tubulin Deacetylase Inhibitor

In a pilot experiment, 352 hydroxamic acids from our HDAC-biased 1,3-dioxane library were screened in a pair of assays designed to identify molecules that selectively inhibit either histone deacetylation or tubulin deacetylation. One compound, JCWII114 (shown below), potently and selectively inhibited tubulin deacetylation but had no visible effect on histone deacetylation.

Structure of JCWII114, a selective tubulin deacetylase inhibitor:

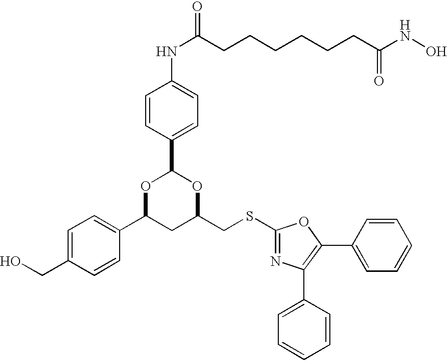

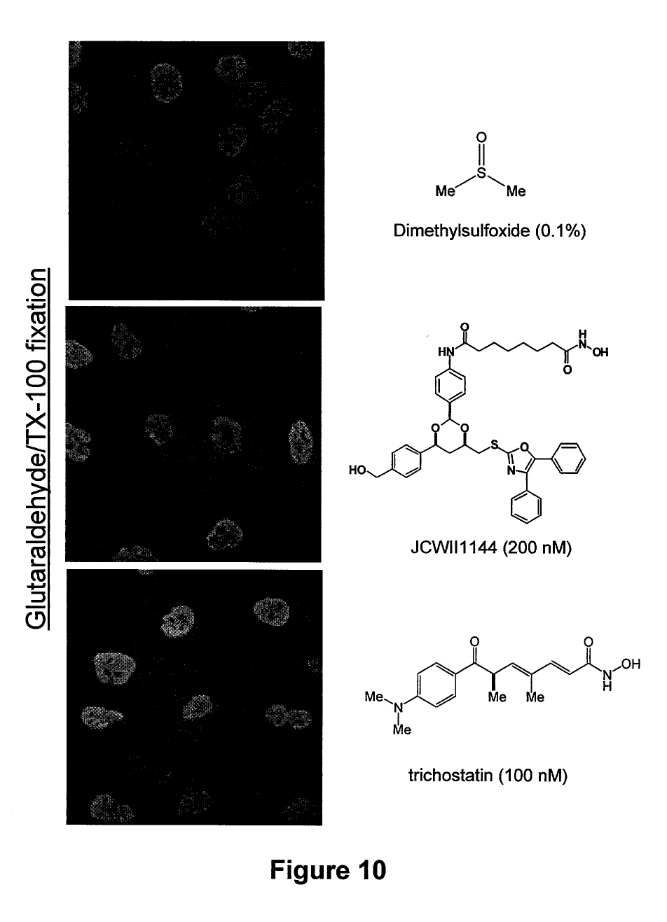

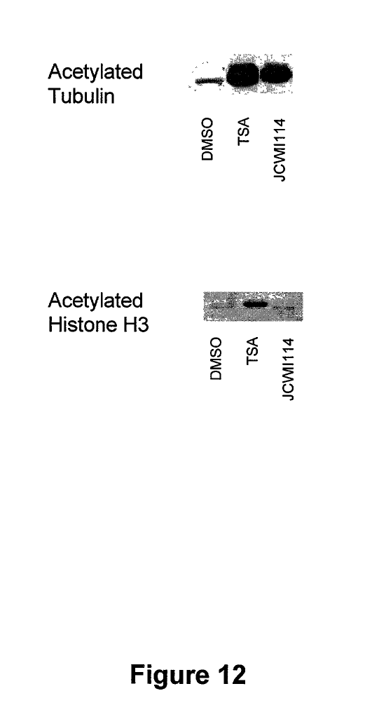

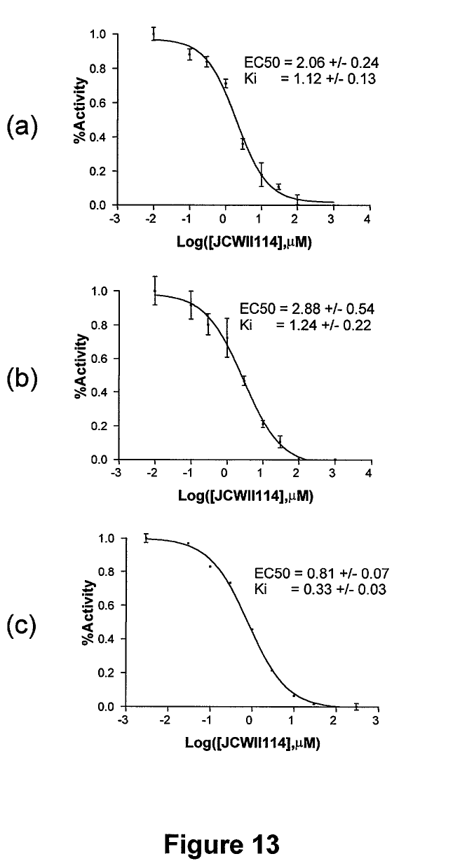

This result was confirmed by immunofluorescence microscopy (FIG. 10) and Western blot analysis (FIG. 12). Furthermore, in an in vitro enzyme inhibition assay, JCWII114 exhibited 3-fold selectivity for HDAC6 over HDAC1 and HDAC4 (FIG. 13). This, along with other data that implicate HDAC6 as the tubulin deacetylase, suggests that HDAC6 is the target of JCWII114. Studies are underway to confirm that HDAC6 is a tubulin deacetylase and that it is the relevant target of JCWII114.

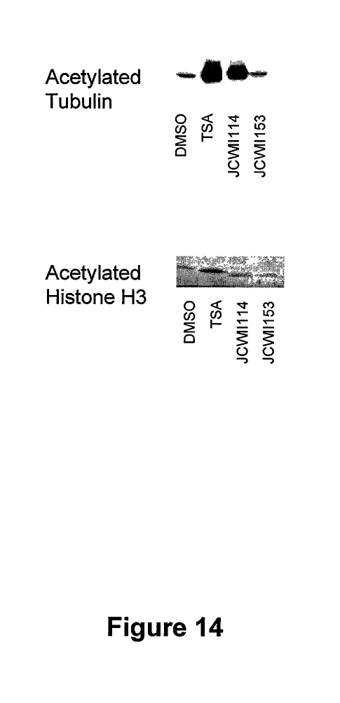

The carboxylic acid analogue of JCWL114 did not affect histone or tubulin deacetylation according to Western blot analysis (FIG. 14). This result both validates our use of the hydroxamic acid functionality to generate an HDAC-biased library, as well as suggests that JCWII114 is, in fact, inhibiting a deacetylase rather than affecting some other pathway that regulates acetylation of tubulin.

Structure of JCWII153, the carboxylic acid analogue of JCWII114.

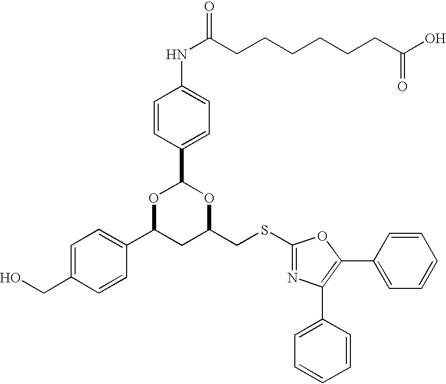

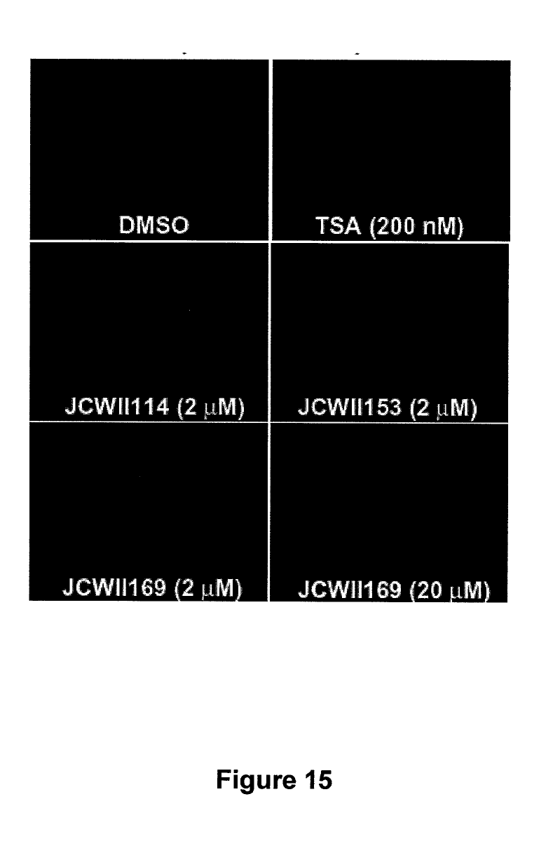

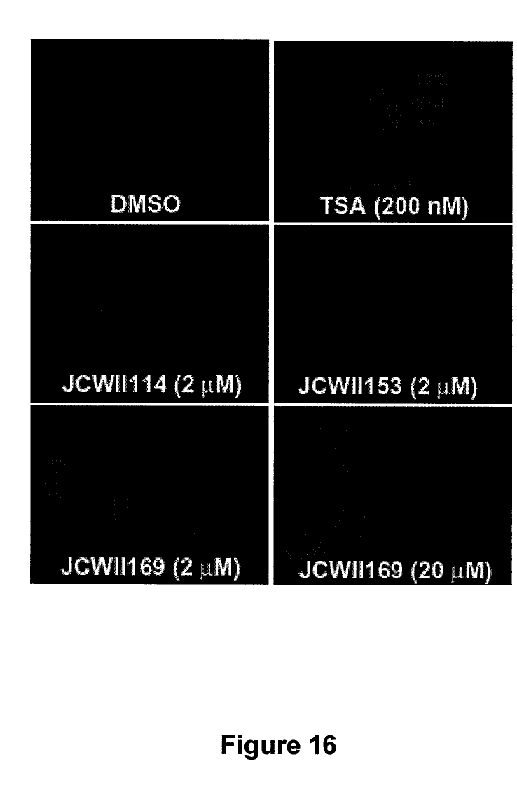

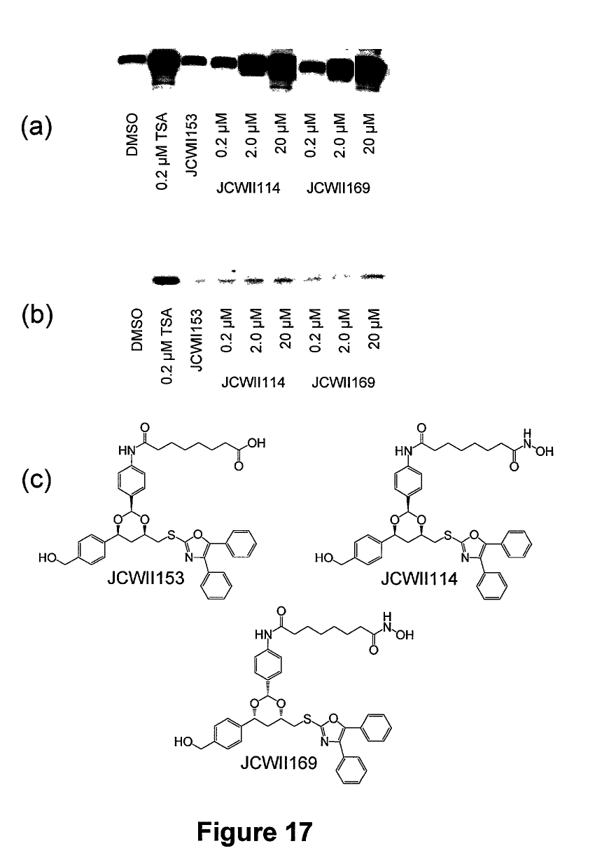

JCWII169, the enantiomer of JCWII114 was analyzed by immunofluorescence microscopy (FIGS. 15 and 16) and Western blot (FIG. 17). No significant difference in selectivity or potency was observed between JCWII169 and JCWII114.

JCWII114 appears to be a truly selective tubulin deacetylase inhibitor. Unlike trichostatin, trapoxin, and other indiscriminate HDAC inhibitors, JCWII114 has no apparent affect on the cell cycle or on cell morphology. Whereas with previous HDAC inhibitors the effects of histone and tubulin deacetylation were intertwined, JCWII114 will allow us to unravel cellular processes directly affected by the acetylation state of tubulin from those affected by the acetylation state of histones.

Screening the Entire HDAC-biased Library for Selective Tubulin and Histone Deacetylase Inhibitors

Based on the success of our pilot screen, we proceeded to assay the full library (2400 hydroxamic acids, 2400 carboxylic acids, and 2400 ortho-amninoanilides) both find additional potent and selective deacetylase inhibitors, and learn what structural elements, if any, are responsible for potency and selectivity. Our data demonstrate unequivocally the importance of the hydroxamic acid functionality in conferring potency.

### II. EXAMPLE 2

### Synthesis of 1,3-dioxanes and use in Multiple Phenotypic and Protein-binding Assays

As described above, the compounds of the invention are useful as inhibitors of HDAC. In addition, small molecules, such as the compounds of the invention, provide a means to modulate rapidly and therefore dissect the circuitry of biological networks (Mitchison et al. Chem. Biol. 1994, 1, 3-6; Schreiber et al. Bioorg. Med. Chem. 1998, 6, 1127-1152). Such compounds can be discovered using phenotypic (Mayer et al. Science 1999, 286, 971-974) or protein-binding assays (MacBeath et al. J. Am. Chem. Soc. 1999, 121, 7967-7968). Phenotypic assays can be used to identify small molecules that modulate a specific cellular or organismic pathway without prior knowledge of the protein components of the pathway. Protein-binding assays, often used in drug discovery efforts, can also be used to identify reagents for exploring protein finction in subsequent biological assays. By determining the pathways and processes altered by the small molecule, the functions of its target can be elucidated. Both strategies are capable of providing insight into complex processes.

The use of small molecules to dissect biological function is being accelerated by high throughput screening of large collections of small molecules (Mayer et al. Science 1999, 286, 971-974). Advances in the use of robotics and the miniaturization of phenotypic and protein binding assays have facilitated rapid screening of large compound collections. However, the production of small molecule libraries has not matched the advances in screening technology. The development of solid phase organic synthesis has increased productivity in organic synthesis through simplification of purification protocols, permitting reactions to be automated and run in parallel (Bunin et al. J. Am. Chem. Soc. 1992, 114, 10997-10998; DeWitt et al. Proc. Natl. Acad. Sci. U.S.A. 1993, 90, 6909-6913). This approach has gained widespread acceptance because milligram quantities of each small molecule can be generated for screening in multiple biological assays; however, issues of cost and labor typically limit library sizes to hundreds of compounds. Millions of distinct compounds can be synthesized through a variation of solid phase synthesis that treats each solid phase particle (commonly, derivatized polystyrene beads) as a separate reaction vessel. By splitting and pooling (Furka et al. Int. J. Pept. Protein Res. 1991, 37, 487-493; Lam et al. Nature 1991, 354, 82-84; Houghten et al. Nature 1991, 354, 84-86) the collection of synthesis beads over a reaction sequence, all possible combinations of a large matrix of building blocks can be accessed, generating an enormous amplification in the number of different compounds produced for a small number of reactions (Tan et al. J. Am. Chem. Soc. 1999, 121, 9073-9087). Despite the introduction of the split-pool synthetic method over a decade ago, it has not gained widespread use due to challenges in compound identification, minute quantities of released compounds, and the resulting tendency to screen molecules as mixtures. By addressing these issues, we have developed a split-pool synthetic approach to generate arrayed stock solutions of single 1,3-dioxane compounds sufficient for multiple phenotypic and protein-binding assays. Synthesis of a small demonstration library of 1890 molecules (a precursor step to a larger 50,000 compound library), structure determination of active compounds, and biological activities in multiple assays are discussed.

A high capacity solid support and a silicon linker enable the synthesis of small molecules in quantities sufficient for multiple phenotypic and protein-binding assays. A fundamental challenge to the production of stock solutions from a split-pool library suitable for multiple biological assays is the release of sufficient compound from the synthesis resin. To generate the concentrations of small molecule necessary for phenotypic assays in cell culture and in multicellular organisms, 5-10 mM stock solutions in 5-10 μL of DMSO or DMF are desirable. When using miniaturized assays (assay volumes 2-40 μL), this permits hundreds of assays to be performed at screening concentrations of up to 100 μM after dilution into an assay plate. To obtain sufficient quantities of arrayed small molecule stock solutions for multiple assays, 500 μm aminomethyl polystyrene beads (Rapp Polymere- Tubingen, Germany) with a loading capacity of 85 nmol/bead were used. These beads have ˜5-fold larger diameter than the commonly used synthesis resin, with ˜100-fold greater quantity of attached small molecule. Synthesis resin with a capacity of 50-100 nmol/bead is sufficient to generate 10 mM stock solutions in 5 μL of DMSO.

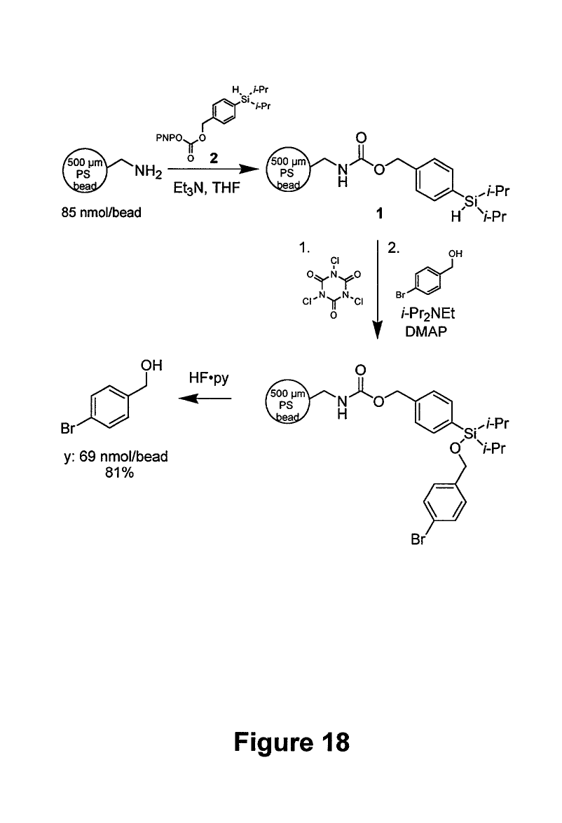

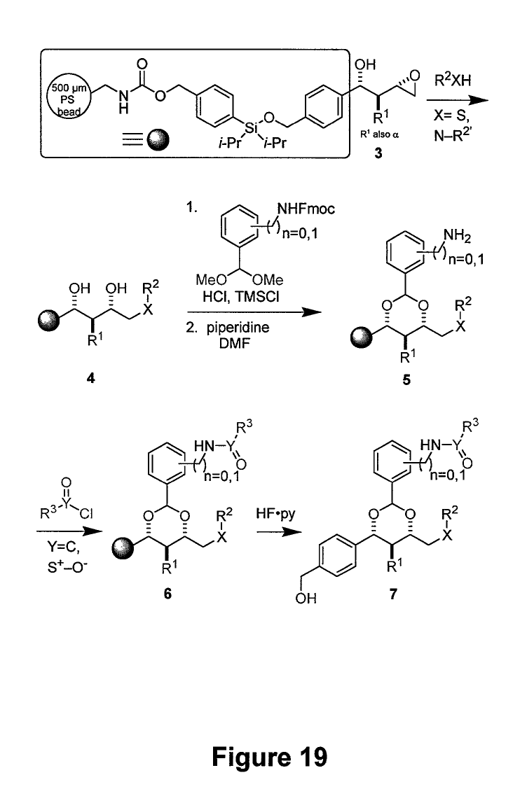

An acid and base-stable diisopropylphenylsilyl ether linker (1) was developed for the 1,3-dioxane synthesis to permit mild fluoride mediated cleavage of the small molecules (FIG. 18). Despite the availability of many acid and base stable linkers (Hu et al. Tetrahedron Lett. 1998, 39, 2711-2714) few of these exhibit stability to both of these sets of reaction conditions as required by our 1,3-dioxane library synthesis (FIG. 19). Additionally, chemically robust linkers typically require harsh cleavage conditions that are not compatible with a wide variety of chemical functionality present in the 1,3-dioxane library. To avoid post-synthetic purification strategies, the reagent used for cleavage of the small molecule at the end of the synthesis should be removed easily, preferably by evaporation, further limiting the possible linker chemistries to be employed. Alkylsilyl ether chemistry was focused on which is widely used in organic synthesis because silyl ethers are often stable to both acid and base, but they are cleaved under mild conditions with fluoride. A common source of fluoride, HF.pyridine (HF.py), can be quenched with TMSOMe yielding volatile byproducts thereby obviating the need for purification after compound cleavage. The diisopropylphenylsilyl linker 1 was developed with these considerations in mind.

The diisopropylphenylsilane linker, activated as a para-nitrophenyl carbonate (2), was attached to aminomethyl polystyrene synthesis resin through a carbamate linkage. Oxidation of silane 1 with trichloroisocyanuric acid generated a silyl chloride that was reacted with alcohol building blocks. To illustrate the intrinsic yield of attachment and release for silyl linker 1, 4-bromobenzyl alcohol was attached and then cleaved with HF.py in 81% yield, releasing 69 nmol/bead on average. This amount is sufficient to prepare ˜10 mM stock solutions by addition of 5-10 μL of DMSO. Due to the development of miniaturized phenotypic and protein binding assays utilizing robotic liquid transfer of 4-40 nL droplets on the tips of specially machined pins (Lipinski et al. Adv. Drug Delivery Rev. 1997, 23, 3-25) these stock solutions can be assayed hundreds of times at 50-100 μM assay concentrations.

Split-pool library synthesis. The discovery of small molecule partners for uncharacterized proteins can provide powerful tools to explore biology. While many guidelines exist for the molecular structure of drugs (Lindsley et al. J. Am. Chem. Soc. 2000, 122, 422-423) the only generally accepted criterion for structures of protein-binding small molecules is that they should contain elements to restrict their degrees of conformational freedom. Bias towards “pharmacophore” or natural product structures can provide inspiration for a synthesis (Schreiber et al. Science 2000, 287, 1964-1969; Bunin et al. J. Am. Chem. Soc. 1992, 114, 10997-10998) but there is no inherent requirement to adhere to these structural motifs for the synthesis of small molecule partners to uncharacterized proteins. Some considerations for such diversity-oriented organic syntheses have been provided.

The 1,3-dioxane structure (FIG. 19) was selected for split-pool synthesis because it is a rigid core that can be synthesized stereoselectively with high purity in the presence of diverse ancillary functional groups. Building blocks for the library were selected through a series of quality control experiments involving liquid chromatography-mass spectrometry (LC-MS) analysis of the building blocks in model reactions on 500 μm polystyrene beads. The building blocks that underwent test reactions with >90% purity were selected for the synthesis. Although our intention is to synthesize a library of 50,000 1,3-dioxane molecules, we first demonstrated our strategy for producing split-pool libraries as arrayed stock solutions by synthesizing a 1890 (theoretical) member library synthesized from a subset of the tested building blocks.

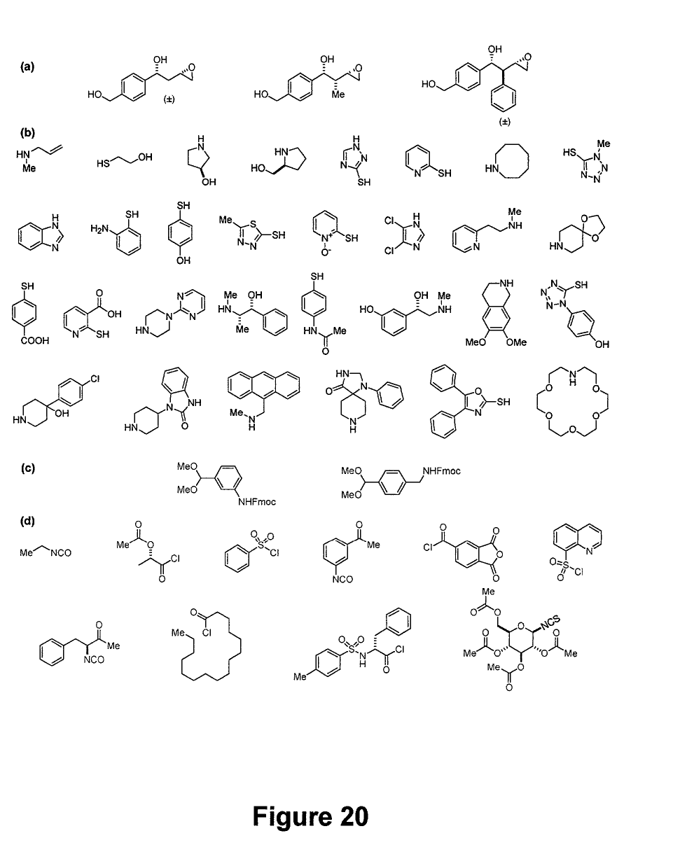

Three γ,δ-epoxy alcohols (FIG. 20) were attached to the polystyrene solid support through the diisopropylphenylsilyl ether linkage in 90% of the theoretical yield (theoretical yield based on loading/cleavage sequence of 4-bromobenzyl alcohol). The epoxy alcohol derivatized resin (3) was pooled and then split into 30 vessels with a diverse set of secondary amine and thiol building blocks (FIG. 20b) to generate 90 different 1,3-diols (4) in quantitative yield, a portion of which were set aside for screening in biological assays. The solid supported 1,3-diols were pooled and split into two portions that were reacted with Fmoc-aminodimethyl acetal building blocks (FIG. 20c) in 0.05 M HCl in dioxane and TMSC1 to furnish 180 Fmoc-amino-1,3- dioxanes in 85-95% yield. The use of TMSC1 as a dehydrating agent was important for consistently driving the reaction to completion. The diisopropylphenylsilyl ether linkage was stable to 0.05 M HCl in anhydrous dioxane for 4 h; however, yield and purity were substantially reduced with higher HCl concentrations or with longer reaction time. Dimethylacetal building blocks were used for 1,3-dioxane formation because the corresponding aldehydes reacted slowly when forming the cis,cis-5-methyl-1,3-dioxanes. This is presumably due to the development of four gauche interactions with the axial C5 methyl group as no difficulties were observed in forming the trans,trans-5-phenyl-1,3-dioxanes which have only two gauche interactions with the C5 phenyl group. Dimethylacetal building blocks led to the unwanted formation of mixed acetals with hydroxyl functionality present in the nucleophile building blocks. Because it was considered desirable to maintain free hydroxyl functionality due to considerations of molecular diversity, these acyclic acetals were removed by treatment of the resin with 0.2 M pyridinium para-toluenesulfonate in 9:1 THF-MeOH. The resin was then pooled and treated with piperidine to effect Fmoc removal, washed with TMSC1 to protect any free hydroxyls, and the solid supported amines (5) were split and reacted with 10 electrophiles (FIG. 20d) to generate 1800 amides, ureas, thioureas, and sulfonamides (6). Two equivalents of aminomethyl polystyrene were used for the synthesis, thus 3780 compounds (3600 1,3-dioxanes and 180 1,3-diols) were synthesized in two days with only 48 reactions.

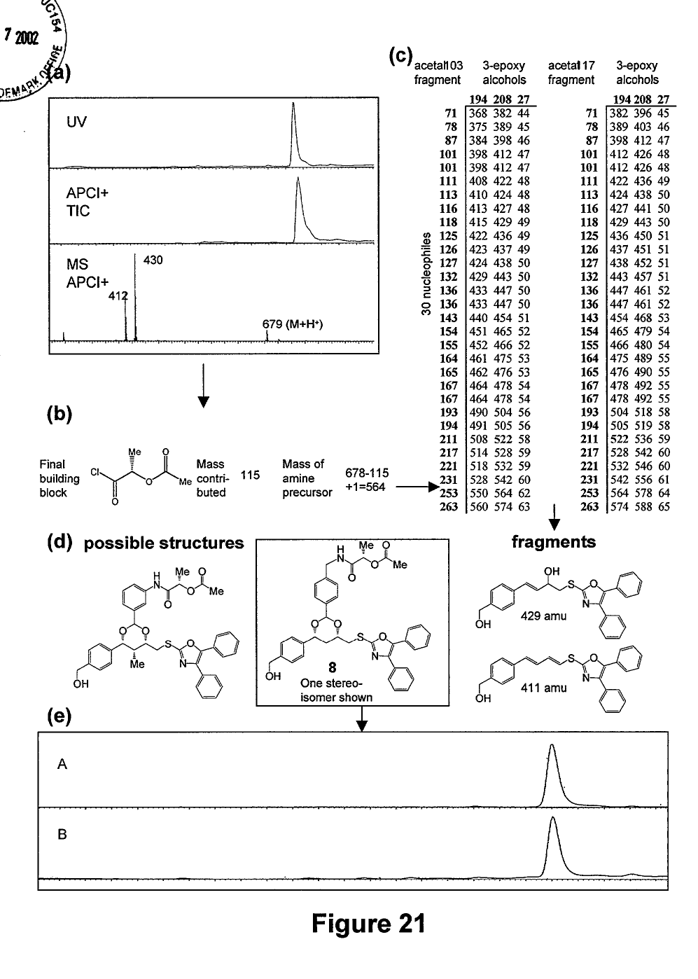

Concentrations of arrayed stock solutions. Traditionally, split-pool libraries are screened as mixtures, requiring deconvolution strategies to identify the active compounds (Erb et al. Proc. Natl. Acad. Sci. U.S.A. 1994, 91, 11422-11426; Freier et al. J. Med. Chem. 1995, 38, 344-352). Mixtures are prone to high false positive and false negative frequency, and they demand considerable labor when identifying the active components from multiple assays. In order to segregate each synthetic compound for the preparation of arrayed stock solutions, the collection of beads with attached 1,3-dioxanes was distributed into eleven 384 well polypropylene plates using a bead arraying tool. The 1,3-dioxanes (7) were released from the beads by treatment with HF.py for 1.5 h followed by TMSOMe to quench the excess HF as volatile byproducts. Solvent evaporation and addition of 5 μL DMSO generated stock solutions of individual compounds. The concentration of a representative stock solution from the library was spectrophotometrically determined to be 6.7 mM by comparison to a standard curve calculated from a purified bulk sample of the relevant compound (8 in FIG. 21).

To study bead-to-bead variability of the amount of released compound, eleven synthesis beads with attached 1,3-dioxane 8 were synthesized, arrayed, cleaved under the above conditions, and analyzed using spectrophotometry. After soaking the beads in 5 μL of DMSO for 14 days, the stock solution concentrations were between 1.9 mM and 10.1 mM (median concentration: 6.2 mM, mean concentration: 5.4 mM). This variance in the concentration is likely the result of small variations in bead diameter which, due to the cubic relationship of diameter to volume, leads to a spread in the amount of compound attached within the bead and, thus, in the concentration of the stock solutions.

Bulk cleavage of the beads used above liberated 59 nmol/bead of compound 8 which was determined by dividing the amount of isolated compound after cleavage, bead washing, and chromatography by the number of beads cleaved. Using this value, the theoretical mean stock solution concentration in 5 μL of DMSO is 11.9 mM. The discrepancy between the theoretical mean concentration of 11.9 mM and the observed mean concentration of 5.4 mM can be attributed to several factors. The lower than expected concentration is due, in part, to the absorption of atmospheric water by the DMSO stock solutions over time leading to an increase in solution volume with a corresponding decrease in concentration. Over 14 days, the stock solutions were observed to undergo a volume increase of 40%. In the future, wet DMSO or solvents that absorb less water will be tested. Correction for the volume increase would give a mean concentration of 7.6 mM, still less than the theoretical mean concentration. The remaining difference in observed versus theoretical concentration is likely due to inefficient extraction of the cleaved compound out of the bead. We have observed that DMSO does not swell polystyrene beads, and extraction of compound remaining in the bead after cleavage may be slow, especially for large diameter beads. The development of miniaturized, highly parallel bead washing procedures or the use of solvents with better swelling properties may diminish this concentration discrepancy.

Despite concentrations below the theoretical value, the dilution of these stock solutions into cell based assays permit, on average, 50 μM screening concentrations at 1% final DMSO concentration. We have found that this is sufficient to discover biologically active molecules in a wide variety of protein-binding and phenotypic assays using the one compound-one bead approach.

Post-synthetic purity analysis of the arrayed stock solutions. The post-synthetic purity of the library was analyzed by liquid chromatography on ten randomly selected stock solutions from each of the ten final acylation reactions (100 beads). Of the selected stock solutions, 47% were >90% pure after 4 synthetic steps and 76% were >70% pure. Incomplete acylation, over-acylation and oxidation were responsible for 23% of the solutions being <90% pure. These inefficiencies with the final acylation step have been addressed by slight alterations in the synthetic method. Specifically, the acylation reaction times have been extended to ensure the completion of this reaction in all cases, and the triethylsilyl protecting group is used instead of trimethylsilyl protection to prevent acylation of ancillary hydroxyl groups in the final step of the synthesis.

It appears that, excluding the final acylation reaction, the molecules were synthesized reliably with high purity (most undesired products could be attributed to the acylation step). Although all of the building blocks used for the acylation reactions were shown to give the desired product with >90% purity in test systems, it is not surprising that reaction performance for the full range of building block combinations yields some compounds with lower than expected purity. This difficulty in predicting reaction success is magnified in a split-pool synthesis during the final diversity steps. Because it is untenable to perform reaction optimization on every reaction in a library synthesis, this post-synthesis quality control analysis will be essential to producing the larger 50,000 compound library with the highest possible purity. These considerations lead us to predict that future libraries based on slight optimizations of this chemistry will show >90% purity for ˜70% of the compounds.

Use of mass spectrometry for molecular structure determination. The structures of compounds in the stock solutions were determined using LC-MS (Brummel et al. Science 1994, 264, 399-402) with atmospheric pressure chemical ionization (APCI) or electrospray ionization (ESI). Direct infusion APCI and ESI-MS analysis was not successful due to competing signals from byproducts derived from extensive manipulations of organic solvents in plastic vessels that are required by the use of HF.py. Liquid chromatography was used to separate the small molecule of interest from these largely polymeric impurities. One difficulty in using mass as a unique identifier for compounds in split-pool libraries is the limited number of integer masses in the range relevant to small molecules (250-1000 amu). Our strategy to decrease the mass redundancy was to segregate the beads from the final 10 synthetic reactions into separate stock plates, thereby reducing the problem of distinguishing between 1800 possible masses to distinguishing between 180 masses.

A representative example of the structure determination procedure is illustrated in FIG. 4. After LC-MS analysis of a portion (5%) of the stock solution, the molecular ion was identified (FIG. 21a). Because the beads were not pooled after the last synthetic step, the mass of the final building block was known. Subtraction of this mass from the molecular ion gave the mass of the 1,3-dioxane intermediate, 5 (FIG. 21b). Comparison to a table of the 180 possible 1,3-dioxane masses (FIG. 21c) generated from all combinations of the epoxy alcohol, nucleophile and acetal fragments allowed, in this case, two possible molecular structures with the same mass to be assigned (FIG. 21d). In most cases, the structures of molecules with redundant integer masses were distinguished by a reliable fragmentation of the 1,3-dioxane ring involving elimination of the aldehyde fragment. The appearance of peaks at 429 amu and 411 amu correspond, respectively, to the products from fragmentation of the 1,3-dioxane ring and from subsequent elimination of water. These fragments can be used to unambiguously distinguish between the two 1,3-dioxane constitutional isomers. The proposed 1,3-dioxane, 8, was synthesized and liquid chromatography showed that a mixture of the synthesized compound and the compound from the stock solution gave only one peak that had an identical retention time with the synthesized compound 8 (FIG. 21e).

Features such as isotope patterns and LC retention times were also used in some cases to resolve redundancies. Mass considerations did not influence the choice of desired building blocks, and some mass redundancies existed even after fragment analysis. In these cases two or more molecules would be synthesized and tested separately. The reliability of this structure determination approach was assessed by identification of a molecule from each of the final ten acylation reactions and resynthesis of the proposed structure. Comparison of the retention times by LC-MS showed that this procedure predicted the correct structure ten out of ten times.

Identification of biologically active 1,3-dioxane molecules and related structures. Arrayed stock solutions of single compounds from a split-pool library permit compounds to be tested individually in multiple assays. Using less than 10% by volume of the 1,3-dioxane stock solutions, we have performed five phenotypic assays and over fifty protein-binding experiments in duplicate. Results of these screens are reported here, but, in most cases, the targets have not yet been pursued. However, these experiments confirm that molecules from the library are entering cells and interacting directly with protein targets, validating our approach to screen from split-pool libraries in multiple assays.

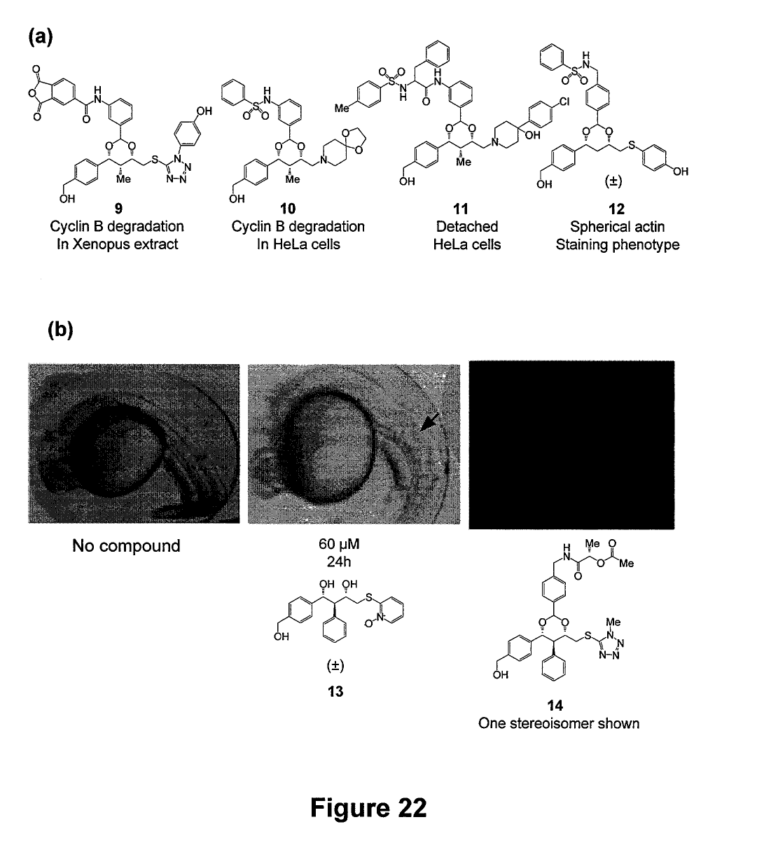

Biologically active 1,3-dioxanes have been identified in phenotypic assays in cell culture, zebrafish, and Xenopus laevis oocyte extract (FIG. 22). Three structurally related phthallic anhydride derivatives (one example, compound 9, is shown) showed inhibitory activity in a miniaturized (4 μL assay volume) Xenopus laevis oocyte extract assay that indicates modulation of the cyclin B degradation pathway (Hughes et al. J Med. Chem. 1998, 41, 3804- 3811). A similar cyclin B degradation assay in HeLa cells (30 μL assay volume) revealed sulfonamide 10 to have inhibitory activity (Note: the use of LC-MS for structure determination is most amenable to moderately sized split-pool libraries (100-5000 compounds). For larger libraries the combined use of LC-MS structure determination and molecular encoding has been utilized). Neither set of compounds was active in both assay systems.

High throughput microscopy of HeLa cells in approximately 2500 single-compound assays using compounds from the 1,3-dioxane library showed cell detachment (induced by 11) and an altered actin staining phenotype (induced by 12). The cellular basis of these phenotypes has not been investigated. No phenotypes consistent with mitotic arrest were observed.

A cytoblot assay in HeLa cells based on the accumulation of phosphorylated nucleolin protein has proven useful for identification of compounds that disrupt mitotic cell cycle progression, including ones that act by novel mechanisms (Mayer et al. Science 1999, 286, 971-974). No molecules in the 1,3-dioxane library led to the accumulation of phosphorylated nucleolin protein. Consistent with observations in the cell staining assays (see above), this indicates that the compounds in the library do not target the mitotic machinery.

To determine the effect of these compounds on whole organism development, a phenotypic assay was performed using 16-cell zebrafish (Danio rerio) embryos (Stockwell et al. Chem. Biol. 1999, 6, 71-83) with 1300 compounds from the 1,3-dioxane library. Embryos treated with a 1,3-diol precursor (13) to the dioxanes at 60 μM developed folds in the anterior trunk region of the notochord at 18 h post-fertilization (FIG. 22b; embryo shown at 24 h post-fertilization for clarity). The folded notochord phenotype has also been observed through genetic mutant screens³² for the gul^{m208} and lev^{m531} mutations. The 1,3-diol (13) may target these gene products or other proteins involved in the same biological pathway. Dissection of the pathways involved in notochord development may be complemented by small molecule ligands for proteins in those pathways.

In addition to phenotypic assays, over fifty protein-binding assays have been performed with the 1,3-dioxane library using small molecule microarrays (MacBeath et al. J. Am. Chem. Soc. 1999, 121, 7967-7968; Peterson et al. Proc. Natl. Acad. Sci. U.S.A. 2000, 97, 12965-12969). The microarrays were constructed by covalent attachment of 1 nIL of each stock solution to a glass slide using a contact printing robot. Labeled proteins were used to probe the microarray for small molecule partners. Small molecules that bind to human FKBP12 (14), histone deacetylase-1, calmodulin, and a variety of fusion proteins derived from the yeast proteome and glutathione-S-transferase have been identified (Stemple et al. Development 1996, 123, 117-128).

### IV. EXPERIMENTALS FOR EXAMPLE 2

Diisopropylphenylsilane resin (1). PS AM NH₂ resin (1.5 g, 1.41 mmol, 1.0 equiv.) was placed in a 20 mL fritted polypropylene tube. A solution of carbonate, 2, (1.7 g, 4.4 mmol, 3.1 equiv.) in THF (12 mL) with Et₃N (0.558 mL, 4.0 mmol, 4.0 equiv.) was added. The reaction was allowed to proceed for 48 h with rotary mixing. The yellow resin was filtered and washed extensively with H₂O and THF until the resin was white to yield diisopropylphenylsilane resin (1).

Representative procedure for γ,δ-epoxy alcohol resins (3). Diisopropylphenylsilane resin, 1, (0.48 g, 0.37 mmol, 1.0 equiv.) was placed in an oven-dried 25 mL round bottom flask under Ar. To remove atmospheric water from the resin, it was washed with anhydrous THF (3×10 mL over 30 min) followed by anhydrous CH₂Cl₂ (2×10 mL over 20 min). A small bore cannula was used to remove the solvents. The resin was suspended in CH₂Cl₂ (3.0 mL) and trichloroisocyanuric acid (0.115 g, 0.5 mmol, 1.4 equiv.) was added. After 1 h a white precipitate had developed and the resin was filtered via cannula and washed with THF (2×10 mL) followed by CH₂Cl₂ (2×10 mL). γ,δ-Epoxy alcohol (0.82 mmol, 2.2 equiv.) was azeotropically dried (4×toluene) and dissolved in CH₂Cl₂ (2.5 mL) with i-Pr₂NEt (0.14 mL, 0.82 mmol, 2.2 equiv.) and DMAP (0.019 g, 0.15 mmol, 0.4 equiv.). The solution was added to the activated resin and allowed to stand for 4 h. The resin was washed 3×DMF and 5×THF to give γ,δ-epoxy alcohol resin (3).

1,3-diol resin (4). γ,δ-Epoxy alcohol resin, 3, were pooled, suspended in DMF (15 mL), and mixed on a rotary shaker for 1.5 h followed by mixing in THF (15 mL) for 2 h. The resin was filtered and dried under vacuum. The dried resin (17 mg, 0.63 mequiv./g avg, 0.011 mmol, 1.0 equiv.) was split into thirty 0.5 dram glass Wheaton vials. To each of the thirty resin portions was added the appropriate nucleophile (0.2 mmol, 18 equiv.) followed by i-PrOH (0.2 mL). In the case of thiols or amine hydrochloride salts, one equivalent of i-Pr₂NEt (35 μL, 0.2 mmol, 18 equiv.) was added and the vials were flushed with Ar, capped, and allowed to stand in a 50° C. oven for 24 h. The reactions were filtered and washed 15×DMF 15×THF to give 1,3-diol resin (4). Approximately six beads from each reaction were set aside for biological assays.

1,3-Dioxane resin (5). After pooling, 1,3-diol resin, 4, was split into two equal portions (0.28 g, 0.58 mequiv./g avg, 0.162 mmol, 1.0 equiv.) and treated with Fmoc-amino dimethyl acetal building blocks (3.2 mmol., 20 equiv.) in a solution of 0.05 M HCl in anhydrous 1,4-dioxane (4.65 mL) and TMSC1 (0.24 mL, 1.9 mmol, 12 equiv.). After 4 h, the reaction was quenched with anhydrous 2,6-lutidine (2 mL), filtered, and washed 4×DMF and 4×THF. The resin was treated with 0.2 M pyridiniumpara-toluenesulfonate in 10% MeOH-THF (2×10 mL) for 2 h. The resin was pooled and treated with 20% piperidine-DMF (3×15 mL) for 15 min and then washed 5 x THF and dried under vacuum. To the resin in CH₂Cl₂ (10 mL), a solution of TMSC1 (0.22 mL, 1.7 mmol, 10 equiv.) and i-Pr₂NEt (0.44 mL, 2.5 mmol, 15 equiv.) in CH₂Cl₂ (5 mL) was added and mixed on a rotary shaker for 1 h. The resin was washed with 10% MeOH-THF for 1 h followed by 5 x THF to give 1,3-dioxane resin (5).

Acyl-1,3-dioxane resin (6). 1,3-Dioxane resin, 5, was split in ten equal portions (0.06 g, 0.54 mequiv./g avg, 0.032 mmol, 1 equiv.) into 1 dram Wheaton vials and suspended in CH₂Cl₂ (acid and sulfonyl chlorides: 2 mL; isocyanates and isothiocyanate: 1 mL) with 2,6-lutidine (acid and sulfonyl chlorides: 0.07 mL, 0.6 mmol, 19 equiv.; isocyanates and isothiocyanate: none added). The electrophile was added (acid and sulfonyl chlorides: 0.4 mmol, 12 equiv.; isocyanates and isothiocyanate: 1 mmol, 31 equiv.) and the reactions were mixed on a rotary shaker (acid and sulfonyl chlorides: 6 h; isocyanates and isothiocyanate: 12 h). The reactions were washed 5×DMF and 5×THF and dried under vacuum to give acyl-1,3-dioxane resin (6).

Resynthesis of 1,3-dioxanes was performed by adaptation of the above procedure

(αS)2-acetoxy)-N-[[4-[(4S,6R)-4-[[(4,5-diphenyl-2-oxazolyl)thio]methyl]-6[4-(hydroxymethyl)phenyl]-1,3-dioxan2-yl]phenyl]methyl-propionamide (8)

¹H NMR (500 MHz, CD₃COCD₃): δ7.76 (s, 1H), 7.64 (dd, 2H, J=8.3, 1.5), 7.56 (dd, 2H, J=8.3, 2.0), 7.48 (d, 2H, J=8.3), 7.46-7.34 (m, 8H), 7.25 (d, 2H, J=8.3), 5.85 (s, 1H), 5.11 (q, 1H, J=6.8), 5.08 (dd, 1H, J=11.2, 2.4), 4.62 (d, 2H, J=6.1), 4.51 (m, 1H), 4.39 (m, 2H), 4.17 (t, 1H, J=6.1), 3.67 (dd, 1H, J=13.7, 4.4), 3.56 (dd, 1H, J=13.7, 7.3), 2.17 (dt, 1H, J=13.2, 2.4), 1.84 (dd, 1H, J=13.2, 11.2), 1.41 (d, 2H, J=6.8). ESI-HRMS m/z calcd for C₃₁H₃₁NO₆S₂Na, found.

(±)-N-[3-[(4S,5R,6R)-4-(1,4-dioxa-8-azaspiro [4.5]dec-8-ylmethyl)6-[4-(hydroxymethyl)phenyl](-5-methyl-1,3-dioxan-2-yl]phenyl-benzenesulfonamide (10):

¹H NMR (500 MHz, CDCl₃): δ7.78 (d, 2H, J=8.3), 7.55 (t, 1H, J=7.8), 7.45 (t, 2H, J=7.8), 7.4-7.2 (m, 7H), 7.04 (d, 1H, J=7.3), 6.81 (s, 1H), 5.71 (s, 1H), 5.08 (s, 1H), 4.77 (d, 1H, J=8.3), 4.70 (s, 2H), 4.00 (d, 2H, J=5.4), 3.98 (obs, 1H), 3.97 (d, 2H, J=5.4), 3.65 (d, 1H, J=12.2), 3.32 (d, 1H, J=13.2), 3.2-3.0 (m, 3H), 2.25 (td, 2H, J=13.2, 3.4), 1.96 (q, 1H, J=6.4), 1.87 (m, 2H), 0.68 (d, 3H, J=6.4). ESI-HRMS m/z calcd for C₃₂H₃₉N₂O₇S 595.2478, found 595.2501.

(αS)-N-[3-[(4S,5R,6R)-4-[[4-(4-chlorophenyl)-4-hydroxy-1-piperidinyl]methyl]-6-[4-(hydroxymethyl)phenyl]5-methyl-1,3-dioxan-2-yl]phenyl-α-[[(4-methylphenyl)sulfonyl]amino]-benzenepropanamide (11)

¹H NMR (500 MHz, CD₃COCD₃): δ9.45 (br s, 1H), 7.76 (br s, 1H), 7.61 (d, 2H, J=8.3), 7.60 (obs, 1H), 7.55 (m, 1H), 7.33 (m, 9H), 7.17 (m, 711), 7.14 (br s, 1H), 5.82 (s, 1H), 5.22 (s, 1H), 4.75 (m, 1H), 4.64 (s, 2H), 4.22 (m, 1H), 3.2-2.8 (m, 8H), 3.11 (dd, 1H, J=13.7, 6.3), 3.09 (dd, 1H, J=13.7, 8.3), 2.21 (s, 3H), 2.13 (br q, 1H, J=6.8), 1.81 (m, 2H), 0.74 (d, 3H, J=6.8). ESI-HRMS m/z calcd for C₄₆H₅₁ClN₃O₇S 824.3136, found 824.3142.

(±)-N-[[4-(4R,6S)-4-[4-(hydroxymethyl)phenyl]-6-[[4-hydroxyphenyl)thiop]methyl]-1,3-dioxan-2-yl]phenyl]methyl-benzenesulfonamide (12)

¹H NMR (500 MHz, CD₃COCD₃): δ8.51 (s, 1H), 7.89 (d, 2H, J=7.3), 7.64 (t, 1H, J=4.9), 7.59 (d, 2H, J=7.3), 7.43 (d, 2H, J=8.1), 7.35 (m, 6H), 7.27 (d, 2H, J=8.1), 6.94 (t, 1H, J=6.3), 6.82 (d, 2H, J=8.3), 5.74 (s, 1H), 4.97 (dd, 1H, J=11.2, 2.4), 4.62 (d, 2H, J=6.3), 4.16 (obs t, 1H, J=6.3), 4.15 (obs, 1H), 4.14 (d, 2H, J=6.3), 3.12 (dd, 1H, J=13.7, 6.8), 2.99 (dd, 1H, J=13.7, 5.9), 2.13 (dt, 1H, J=13.2, 2.4), 1.65 (app q, 1H, J=13.2). ESI-HRMS m/z calcd for C₃₁H₃₁NO₆S₂Na 600.1491, found 600.1515.

(±)-(1R,2S,3S)-1-[4-(hydroxymethyl)phenyl]-4-[(1-oxido-2-pyridinyl)thio]-2-phenyl-1,3-butanediol (13)

¹H NMR (500 MHz, CD₃OD): δ8.27 (d, 1H, J=8.2), 7.39 (td, 1H, J=10.1, 1.8), 7.19 (m, 2H), 7.12 (m, 6H), 7.03 (dd, 2H, J=10.1, 1.8), 5.24 (d, 1H, J=11.4), 4.53 (td, 1H, J=9.6, 3.4), 4.47 (s, 2H), 3.28 (t, 1H, J=11.4), 3.07 (dd, 1H, J=16.7, 3.4), 2.82 (dd, 1H, J=16.7, 9.6). ESI-HRMS m/z calcd for C₃₂H₃₆N₅O₆S, found.

(αS)-2-acetyloxy)-N-[[4-[(4R,5S,6S)-4-[4-(hydroxymethyl)phenyl]-6-[[4,5-diphenyl-2-oxazolyl)thio]methyl]61,3-dioxan-2-yl]phenyl]methyl-propionamide (14)

¹H NMR (500 MHz, CD₃COCD₃): δ7.81 (br s, 1H), 7.45 (d, 2H, J=7.8), 7.31 (d, 2H, J=8.3), 7.24 (m, 9H), 6.10 (s, 1H), 5.23 (d, 1H, J=10.3), 5.12 (q, 1H, J=6.8), 4.74 (qd, 1H, J=8.2, 2.9), 4.52 (d, 2H, J=6.8), 4.43 (m, 2H), 4.10 (t, 1H, J=5.9), 3.92 (s, 3H), 3.45 (dd, 1H, J=13.6, 2.9), 3.38 (dd, 1H, J=13.6, 8.2), 3.19 (t, 1H, J=10.3), 2.09 (s, 3H), 1.42 (d, 3H, J =6.8). ESI-HRMS m/z calcd for C₃₂H₃₆N₅O₆S 618.2386, found 618.2410.

Bead cleavage and formation of arrayed stock solutions. Acyl-1,3-dioxane resin (6) was distributed into ten 384 well polypropylene plates (Corning Costar, 40 μL well volume) using a bead arraying tool to give a single bead per well. A solution of 5% HF.py in THF (15 μL) was added to each well and sealed with foil for 1.5 h. Methoxytrimethylsilane (5 μL) was added using the Hydra 384 liquid transfer instrument (Robbins Scientific) equipped with Teflon coated needles. The solvent was evaporated and the Hydra 384 was used to add DMSO (5 μL) to the wells.

Use of mass spectrometry for molecular structure determination. LC-MS analysis was performed on a Micromass Platform LCZ-MS coupled to a Waters 2690 HPLC. Analyses were run using either an APCI or an ES interface with positive-negative ionization mode switching. Chromatography was over a 3.5 μm Waters Symmetry C18 column (50 mm ×2.1 mm i.d.) eluting at 0.4 mL/min with a gradient of 15-100% B over 10 min (A=water +0.1% formic acid; B=acetonitrile+0.1% formic acid). An aliquot (0.25 μL) of the DMSO stock solution was removed and added to CH₃CN (15 lL) in a glass autosampler vial insert. A 5 μL sample of the solution was injected.

### V. EXAMPLE 3

### Use of 1,3-dioxanes as modulators of a glucose-sensitive subset of genes downstream of Ure2p

The progress in identifying and expressing all human proteins (Wiemann et al. Genome Res. 2001, 11, 422-435) presents an opportunity to develop a small-molecule modulator for every protein function. Small molecule approaches to study protein function have illuminated diverse fields of biology. Examples include tetrodotoxin, which enabled the dissection of the action potential (Narahashi et al J. Gen. Physiol. 1964, 47, 965-974), and agonists of peroxisome-proliferator-activated receptorγ such as rosiglitazone, which illuminated the regulation of adipogenesis (Lehmann et al. J. Biol. Chem. 1995, 12953-12956). However, in most cases no small molecule that can modulate the function of a protein of interest is known, and there is currently no efficient method of identifying these biological probes. Using the example of the yeast protein Ure2p, general two-step method has been demonstrated that does not require a high-resolution structure or a previously characterized small molecule known to bind the protein. First, diversity oriented synthesis is used to produce structurally complex and diverse small molecules efficiently. Second, the resulting compounds are screened for their ability to bind a protein of interest by using small-molecule microarrays, a technique for extremely high-throughput parallel-binding assays. Cell-based studies can subsequently determine which functions of the protein are modulated by each small molecule.

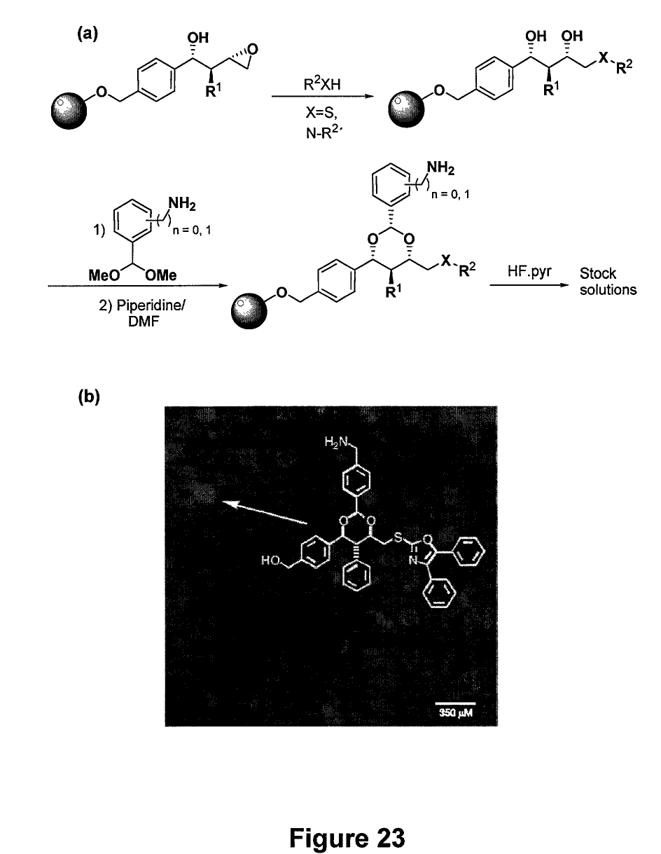

The yeast protein Ure2p has been widely studied in several different contexts. Ure2p is the central repressor of genes involved in nitrogen metabolism (Coschigano et al. Mol. Cell Biol. 1991, 11, 822-832), is capable of switching to a prion form (Wickner et al. Science 1994, 264, 566-569), and is part of a signalling cascade downstream of the Tor proteins (Hardwick et al. Proc. Natl. Acad. Sci. U.S.A. 1999, 96, 14866-14870; Cardenas et al. Genes Dev. 1999, 13, 3271-3279). Because there is no known small molecule that binds to Ure2p, a collection of 3,780 structurally complex 1,3-dioxane small molecules was screened resulting from a diversity-oriented synthesis (See, FIG. 23a). The molecules are structurally unbiased towards any particular protein target and can be used to identify specific probes for many different proteins. This collection of molecules had been prepared with a ‘one bead-one stock’ solution technology platform with the use of macrobeads (FIG. 23a) followed by automated compound cleavage and the generation of 5-mM stock solutions in 5 μl of N,N-dimethylformamide (Sternson et al. J. Am. Chem. Soc. 2001, 123, 1740-1747; Blackwell et al. Chem. Biol. 2001, 8, 1167-1182; Clemons et al. Chem. Biol. 2001, 8, 1183-1195). The small molecules were arrayed in high-density on glass slides (˜800 spots/cm², 1 nl of each compound per slide) with a quill-pin contact-printing robot. These microarrays were probed with fluorescently labeled Ure2p, enabling the protein-binding properties of each molecule to be tested in parallel with minimal protein consumption (protein solution: 20 mg/ml, 0.2 ml). This method has been used to detect known interactions such as that between FKPB12 and a synthetic pipecolyl a-ketoamide and is applied here to the identification of novel small molecule-protein interactions with compounds. Eight compound spots on the microarrays showed reproducible binding to labelled Ure2p (see FIG. 23b for one such spot in an 8×8 spot array where each spot is derived from a single-compound stock solution derived from the diversity oriented synthesis).

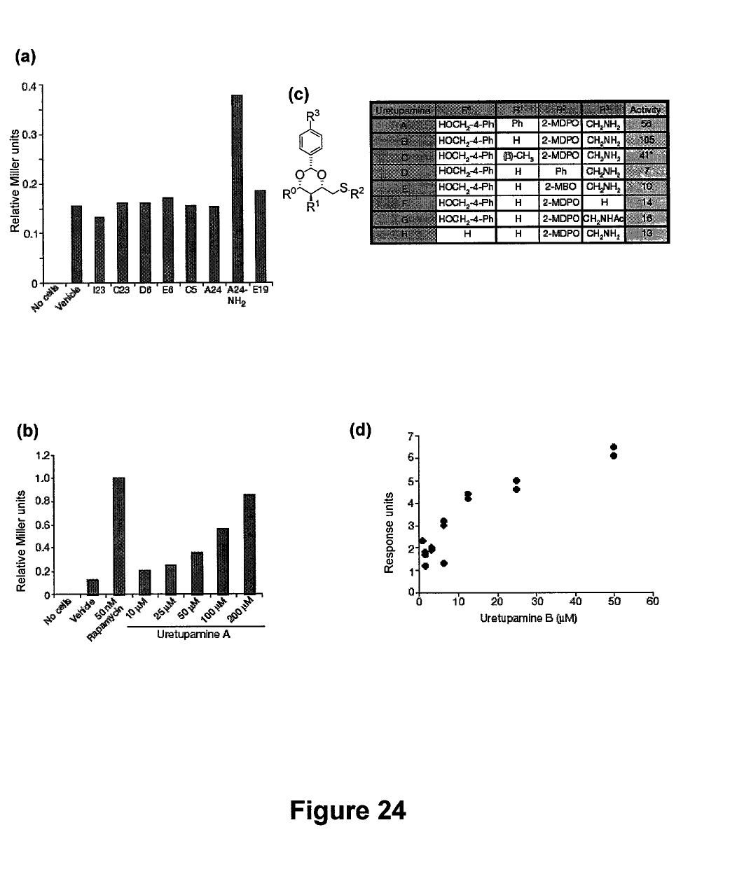

To determine cellular activity, the molecules comprising these spots were resynthesized and tested for the modulation of endogenous Ure2p function with a PUT1-lacZ reporter system because PUT1 expression is known to be repressed by URE2. In addition to the positive control of rapamycin, one of the eight compounds activated this reporter (FIG. 24a). The compound, which was named uretupamine A, gave a concentration dependent dose response that at higher concentrations approached the levels of reporter gene activation induced by rapamycin (FIG. 24b).

To explore structure-activity relationships, a series of compounds were synthesized with systematic variations in the structure of uretupamine A (FIG. 24c). Specific atomic interactions are believed to be responsible for uretupamine binding because most modifications of its structure resulted in a complete loss of activity (FIG. 24c). Uretupamine A was rendered functionally inactive by acylation of the primary amine, replacement of the diphenyloxazole moiety with a phenyl group or a benzoxazole group or modification of the benzyl alcohol moiety (FIG. 24c). However, the C-5 position of the dioxane ring was tolerant to the modification. Because the potency of uretupamine A was attenuated by poor solubility at higher concentrations in yeast medium, a more soluble derivative was synthesized, uretupamine B, which lacked the C-5 phenyl group on the dioxane ring (FIG. 24c). As expected uretupamine B was more potent than uretupamine A (FIG. 24c). Surface plasmon resonance was used to obtain a binding constant for uretupamine A and B binding to purified Ure2p. This demonstrated that uretupamine A and B bound to Ure2p with equilibrium dissociation constants of 18.1 and 7.5 μM, respectively (FIG. 24d), which is consistent with their potencies in cells.

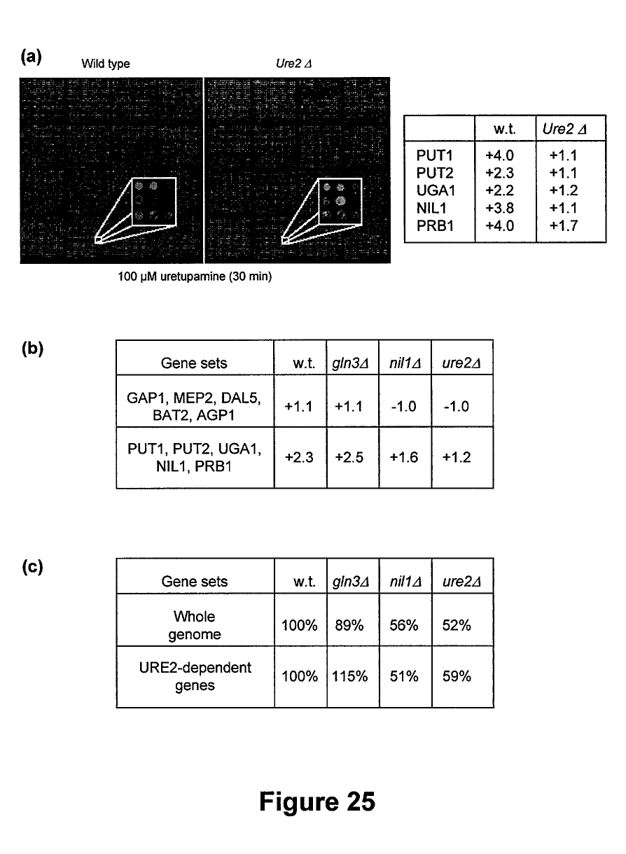

To determine the precise effects and specificity of uretupamine, whole-genome transcription profiling was used in wild-type cells as well as an otherwise isogenic ure2D strain-a ‘targetless’ strain (Marton et al. Nature Med. 1998, 4, 1293-1301). (Complete transcription profiling data are publicly available at http://www.schreiber.chem.harvard.edu) Both uretupamine A and B upregulated only a subset of genes (including PUT1, PU72, PRB1, NIL1 and UGAI1) known to be under the control of Ure2p (FIG. 25a,b). The expression of other genes (including GAP1, MEP2, AGP1, BAT2 and DAL5) controlled by Ure2p was essentially unchanged (FIGS. 25a, b). The compounds had little or no effect on either set of genes in a targetless ure2A strain, an otherwise identical strain lacking only the gene encoding the putative protein to which uretupamines A and B bind (FIGS. 25a, b). This result suggests that a small molecule readily obtained from diversity-oriented synthesis and screening with the use of small- molecule microarrays has nearly complete cellular specificity for its screening partner, at least as judged by its global effects on the MRNA levels of treated cells.

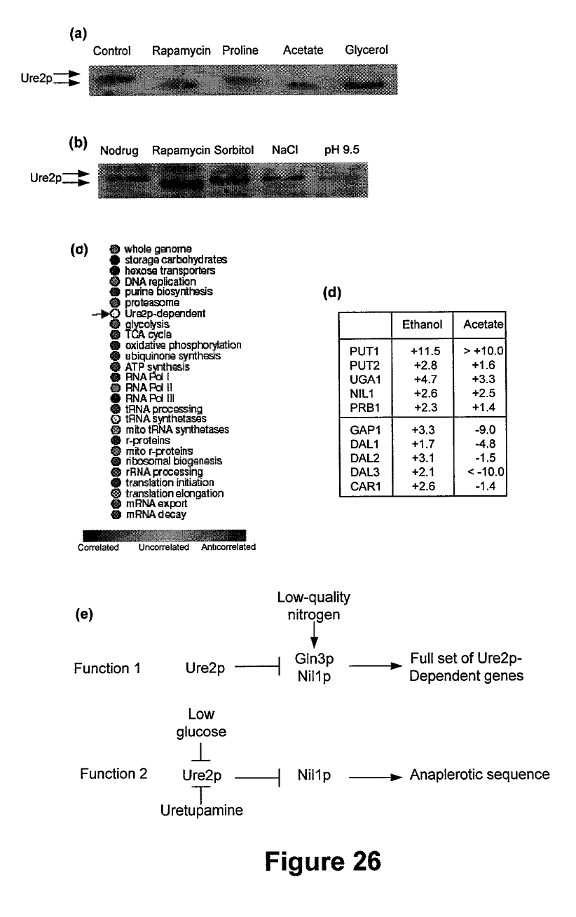

Although Ure2p-controlled genes are normally thought of as responsive to nitrogen quality, the subset of genes induced by uretupamine (PUT1, PUT2, PRB1, NIL1, and UGA1) has been shown to be upregulated when glucose is removed from the media. The mechanism for this differential regulation of Ure2p-controlled genes in response to different nutrient signals is not understood. Ure2p represses transcription factors Gln3p and Nil1p, which might be differentially regulated to achieve this effect. To test this hypothesis, uretupamine B was profiled in gln3Δ and nil1Δ strains. Remarkably, it was found that deleting GLN3 had little effect on the actions of uretupamine , whereas deleting NIL1 abrogated its actions (FIG. 26b). Further confirmation for this selectivity comes from whole-genome vector-based comparisons of four profiles; these comparisons show that URE2 and NIL1, but not GLN3, are critical for uretupamine action (FIG. 25c). Northern blot analysis confirmed the effect of uretupamine B on PUT1 expression (normalized to ACT1 expression) in wild-type, gln3Δ and nil1Δ strains.

The fact that the binding of uretupamine to Ure2p induces the expression of glucose-sensitive genes in a NIL-1 dependent manner suggests that Ure2p might itself be the target of a glucose-sensitive pathway. This is in contrast to a glucose-sensitive pathway impinging on Nil1p, bypassing Ure2p.

Because Ure2p is a phosphoprotein, the phosphorylation state of Ure2p was examined after different types of nutrient shift. Wild-type cells were shifted from the high-quality nitrogen source, ammonium sulphate, to the low quality nitrogen source proline. Cells were also shifted from the high-quality (fermentable) energy source glucose to the low-quality (non-fermentable) energy sources acetate or glycerol. Surprisingly, Ure2p was not dephosphorylated when ammonium sulphate was removed but was dephosphorylated when glucose was removed (FIG. 26a). These data indicate that signals not previously thought to regulate Ure2p after its phosphorylation state, whereas signals previously throught to regulate Ure2p do not alter its phosphorylation state. Idential results were obtained for cells transferred from a glucose-containing medium and for cells of a different background (W303). Other stresses (such as IM NaCl, 1 M sorbitol, pH 9.5 or heat shock) known to upregulate Ure2p-dependent genes did not cause Ure2p dephosphorylation (FIG. 26b). These data suggest that Ure2p is part of a signalling pathway that specifically responds to glucose.

Komberg and Krebs first proposed that on energy sources such as acetate, metabolic sequences called anaplerotic are activated to replenish tricarboxylic-acid-cycle intermediates (Kornberg et al. Nature 1957, 179, 988-991). Yease cells growning in acetate-containing media have been shown to accumulate ammonia (Bogonez et al. Biochim. Biophys. Acta, 1983, 733, 234-241), which leads to the following paradox. Ammonia would repress the expression of Ure2p-dependent genes, including those thought to promote survival on acetate as part of an anaplerotic sequence (PUT1, PUT2 and UGA1). It is possible that acetate-induced Ure2p- dephosphorylation protects the anaplerotic sequence from this repression by ammonia. The transcription profile was performed of yeast shifted from glucose to acetate and compared it with that of cells shifted from glucose to ethanol. Genome-wide analysis showed that Ure2p dependent genes were differentially affected by the two transitions (FIG. 26c). Unlike ethanol, acetate caused the downregulation of some Ure2p-dependent genes, but, like ethanol, acetate induced those genes activated by uretupamine (FIG. 26d). Taken together, these data suggest that Ure2p-dephosphorylation stabilizes the induction of genes for an anaplerotic sequence when other cellular forces might repress the expression of these same genes (FIG. 26e).

The approach described herein to uncovering the role of Ure2p in glucose signalling is rooted in the principles of reverse genetics. A method of modulating Ure2p function selectively was desired to examine the resulting phenotype. Because uretupamine modulates only a subset of Ure2p function, its effects are more specfiic than those resulting from deletion of the URE2 gene. This property of uretupamine highlights the multifunctionality of individual proteins and addresses the challenge in proteomics to identify and control all possible inputs and outputs of each protein. With uretupamine, a functional connection has been demonstrated between Ure2p, Nillp and glucose levels. A means to control this system more selectively than any physiological stimulus or genetic deletion has been demonstrated. Diversity-oriented synthesis and small-molecule microarrays provide a potentially systematic method for acquiring powerful probes, where different small molecules can modulate different aspects of a protein's function, preceding the discovery of a genetic allele of a similar phenotype.

### VI EXAMPLE 4

### In vivo activity

Although a variety of methods can be utilized, one exemplary method by which the in vivo activity of the inventive compounds is determined is by subcutaneously transplanting a desired tumor mass in mice. Drug treatment is then initiated when tumor mass reaches approximately 100 mm after transplantation of the tumor mass. A suitable composition, as described in more detail above, is then administered to the mice, preferably in saline and also preferably administered once a day at doses of 5, 10 and 25 mg/kg, although it will be appreciated that other doses can also be administered. Body weight and tumor size are then measured daily and changes in percent ratio to initial values are plotted. In cases where the transplanted tumor ulcerates, the weight loss exceeds 25-30% of control weight loss, the tumor weight reaches 10% of the body weight of the cancer-bearing mouse, or the cancer-bearing mouse is dying, the animal is sacrificed in accordance with guidelines for animal welfare.

### VII EXAMPLE 5

### Assays to identify potential antiprotozoal compounds by inhibition of histone deacetylase.

As detailed in U.S. Pat. No. 6,068,987, inhibitors of histone deacetylases may also be useful as antiprotozoal agents. Described therein are assays for histone deacetylase activity and inhibition and describe a variety of known protozoal diseases. The entire contents of 6,068,987 are hereby incorporated by reference.

1. A compound having the structure (I): 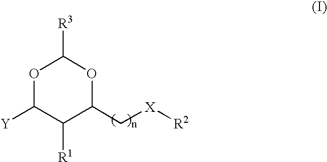
and pharmaceutically acceptable derivatives thereof,
wherein R¹ is hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety;
n is 1-5;
R² is hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety;
X is —O—, —C(R^{2A})₂—, —S—, or —NR^{2A}—, wherein R^{2A} is hydrogen, a protecting group, or an aliphatic, heteroaliphatic, aryl, heteroaryl, —(aliphatic)aryl, —(aliphatic)heteroaryl, -(heteroaliphatic)aryl, or -(heteroaliphatic)heteroaryl moiety;
or wherein two or more occurrences of R² and R^{2A}, taken together, form a cyclic aliphatic or heteroaliphatic moiety, or an aryl or heteroaryl moiety;
R³ is an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety; and
Y is hydrogen or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic groups is independently substituted or unsubstituted, cyclic or acyclic, linear or branched, and each of the foreoing aryl and heteroaryl groups is substituted or unsubstituted:

2. The compound of claim 1, wherein the compound has the structure as shown in formula (Ia):

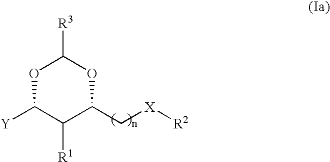

3. The compound of claim 1, wherein Y is an aryl or heteroaryl moiety substituted with Z, wherein Z is hydrogen, —(CH₂)_{q}OR^{A}, —(CH₂)_{q}SR^{Z}, —(CH₂)_{q}N(R^{Z})₂, —(C═O)R^{Z}, —(C═O)N(R^{Z})₂, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, wherein q is 0-4, and wherein each occurrence of R^{Z} is independently hydrogen, a protecting group, a solid support unit, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety.

4. The compound of claim 1, wherein Y is a substituted phenyl moiety and the compound has the structure (II):

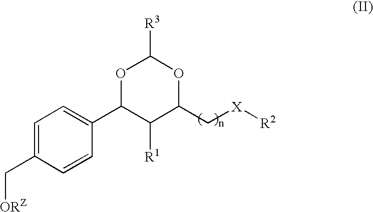

5. The compound of claim 1, wherein Y is a substituted phenyl moiety and X is S and the compound has the structure (III):

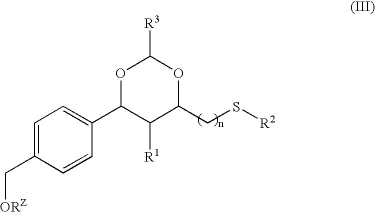

6. The compound of claim 1, wherein Y is a substituted phenyl moiety and X is —NR^{2A} and the compound has the structure (IV):

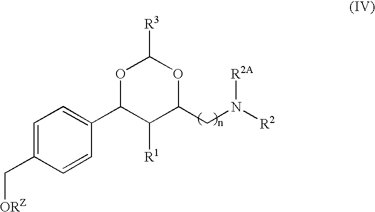

7. The compound of claim 1, wherein Y is a substituted phenyl moiety and X is O and the compound has the structure (V):

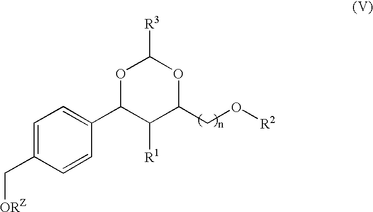

8. The compound of claim 1, wherein Y is a substituted phenyl moiety and R³ is a phenyl moiety substituted with R⁴ and the compound has the structure (VI): 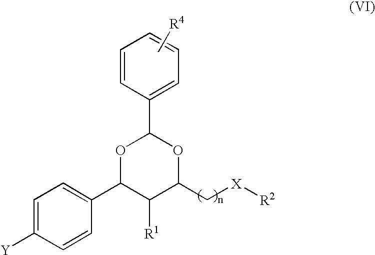
wherein R⁴ is —(CH₂)ᵣN(R^{4A})₂, —CH₂)ᵣSR^{4A}, —(CH₂)₄OR^{4A}, —(CH₂)ᵣNY^{4A}C(═O), —CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, —(heteroalipahtic)aryl, or —(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

9. The compound of claim 1, wherein Y is a substituted phenyl moiety and R³ is a phenyl moiety substituted with R⁴ and the compound has the structure (VII): 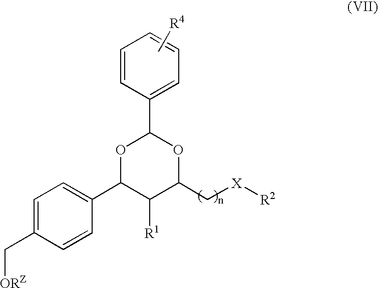
wherein R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, (heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B})₂, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

10. The compound of any one of claims 1-9, wherein R¹ is hydrogen, lower alkyl, or a substituted or unsubstituted phenyl moiety.

11. The compound of any one of claims 1-9, wherein R¹ is hydrogen, methyl, or phenyl.

12. The compound of any one of claims 1-9, wherein R¹ is hydrogen.

13. The compound of any one of claims 1-9, wherein either or both of R², R^{2A}, or R² and R^{2A}, taken together with N, comprise 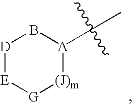
wherein m is 0-3; A-B, B-D, D-E, E-G, G-J, two or more occurrences of J, and J-A are each connected by a single or double bond; A is CH, C, or N; B is CR^{B}, C(R^{B})₂, (C═O), NR^{B}, N, O or S; D is CR^{D}, C(R^{D})₂, (C═O), NR^{D}, N, O or S; E is CR^{E}, C(R^{E})₂, (C═O), NR^{E}, N, O or S; G is CR^{G}, C(R^{G})₂, (C═O), NR^{G}, N, O or S; and each occurrence of J is independently CR^{J}, C(R^{J})₂, (C═O), NR^{J}, N, O or S; wherein each occurrence of R^{B}, R^{D}, R^{E}, R^{G} and R^{J} is independently hydrogen, halogen, hydroxyl, protected hydroxyl, thiol, protected thiol, amino, protected amino, —COOH, —CONH₂, —NHCOOH, —NHCOO(alkyl), —NHCO(alkyl), or a substituted or unsubstituted, cyclic or acyclic, linear or branched alkyl or heteroalkyl moiety, or a substituted or unsubstituted aryl or heteroaryl moiety, or any two or R^{B}, R^{D}, R^{E}, R^{G} or R^{J} taken together comprises a substituted or unsubstituted cyclic aliphatic or heteroaliphatic, moiety or a substituted or unsubstituted aryl or heteroaryl moiety.

14. The compound of any one of claims 1-6, 8 or 9, wherein —X—R² has one of the structures:

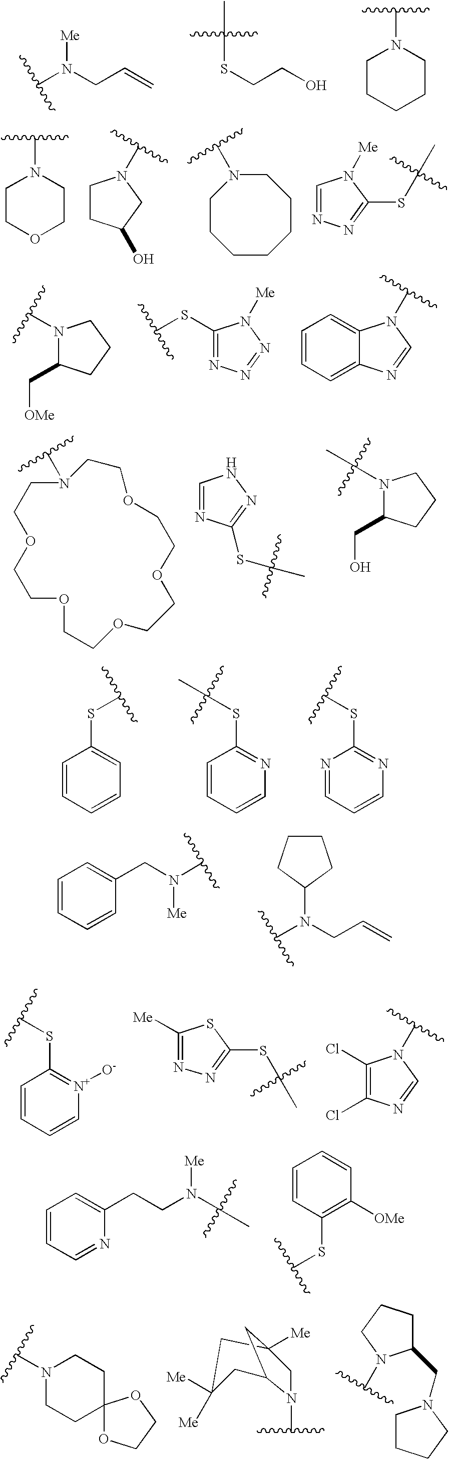

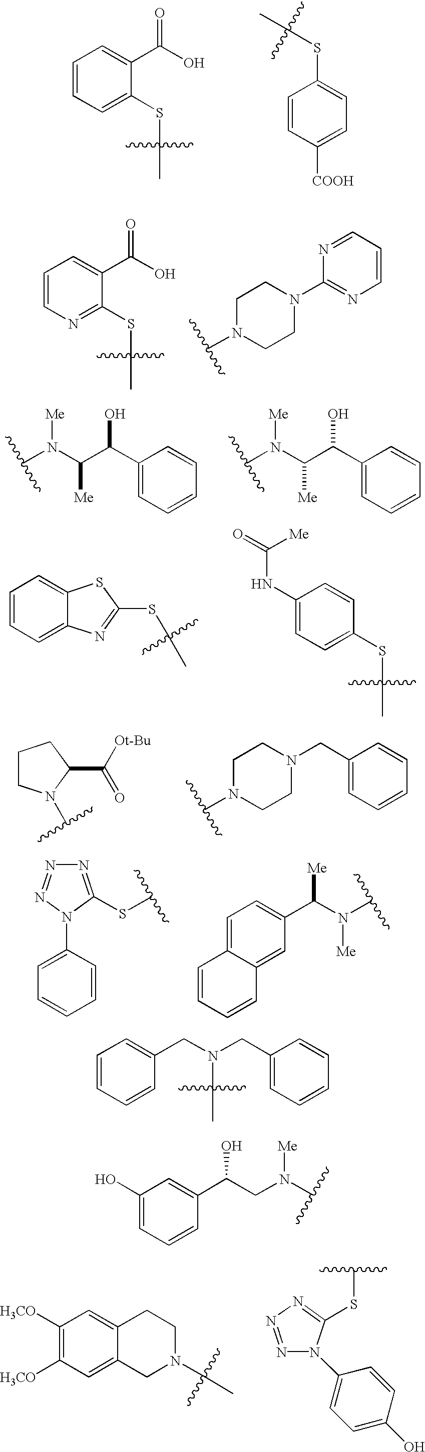

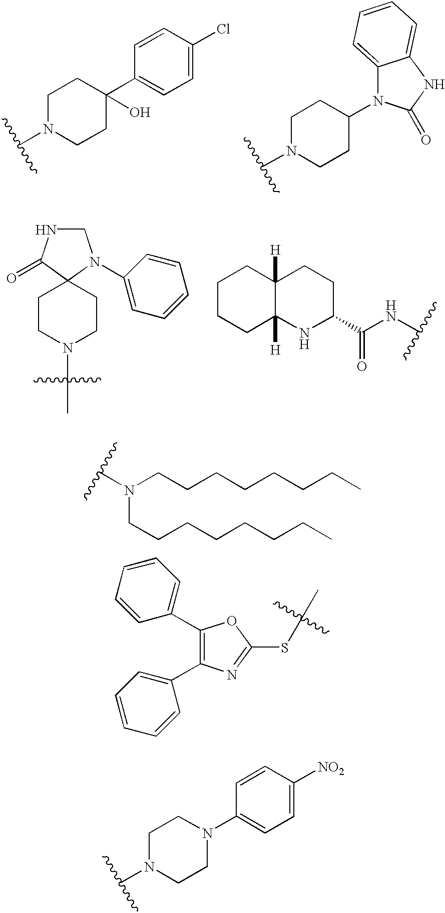

15. The compound of any one of claims 1-9, wherein one or both of R² and R^{2A} is a substituted or unsubstituted aryl or heteroaryl moiety.

16. The compound of any one of claims 1-9, wherein one or both of R² and R^{2A} is an aryl or heteroaryl moiety substituted with —COOH, halogen, alkyl, heteroalkyl, aryl, heteroaryl, OH, SH, NO₂, NH₂, or —NH(C═O)alkyl.

17. The compound of any one of claims 1-7, wherein R³ is a substituted aryl or heteroaryl moiety.

18. The compound of any one of claims 1-7, wherein R³ is an aryl or heteroaryl moiety substituted with —(CH₂)ᵣN(R^{4A})₂, wherein r is 0 or 1 and each occurrence of R⁴A is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, (C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

19. The compound of any one of claims 1-7, wherein R³ is one of the following structures:

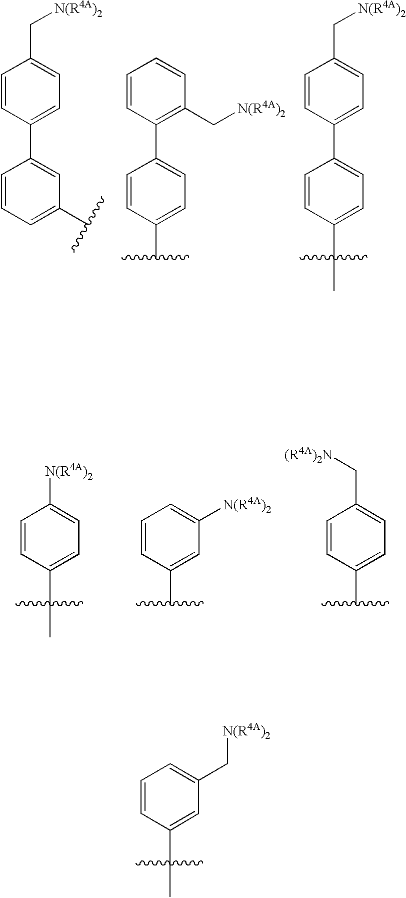

20. The compound of claims 1-9, wherein R^{Z} is hydrogen or a solid support unit.

21. The compound of claim 8 or 9 wherein R⁴ is —(C∇O)(CH₂)ₜ(C═O)NHR^{4B}, wherein R^{4B} hydrogen, a protecting group, hydroxyl, protected hydroxyl, or an alipahtic, heteroaliphatic, aryl, heteroaryl, —(aliphatic)aryl, —(aliphatic)heteroaryl, —(heteroalipahtic)aryl, or —(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

22. The compound of claim 8 or 9 wherein R^{4A} is —(C═O)(CH₂)ₜ(C═O)NHR^{4B}, wherein R^{4B} is hydroxyl and t is 3, 4 or 5.

23. The compound of claim 8 or 9 wherein R⁴A is —(C═O)(CH₂)ₜ(C═O)NHR^{4B}, wherein R^{4B} is hydroxyl, t is 3, 4 or 5, and X—R² is —S—R².

24. The compound of claim 1, wherein the compound has the structure: 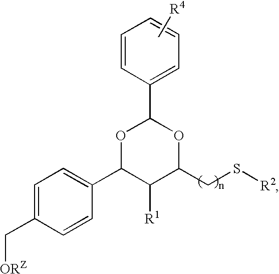
wherein R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, —(aliphatic)aryl, —(aliphatic)heteroaryl, —(heteroalipahtic)aryl, or —(heteroaliphatic)heteroaryl moiety, wherein each occurrence of R^{4A} is independently hydrogen, a protecting group, an aliphatic, heteroaliphatic, aryl, heteroaryl, -(aliphatic) aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

25. The compound of claim 24, wherein R¹ is hydrogen, phenyl or methyl, R^{Z} is hydrogen or a solid support unit; R² is a substituted or unsubstituted alkyl or heteroalkyl moiety, or a substituted or unsubstituted aryl or heteroaryl moiety; and R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

26. The compound of claim 24, wherein R^{4A} is —(C═O)(CH₂)ₜ(C═O)NHR^{4B}, wherein R^{4B} is hydroxyl, t is 3, 4 or 5.

27. The compound of claim 1, wherein the compound has the structure: 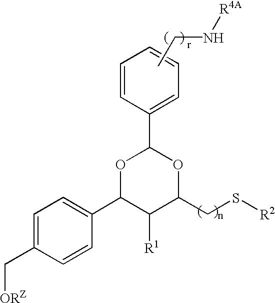
wherein each occurrence of R^{4A} is independently hydrogen, a protecting group, an aliphatic, heteroaliphatic, aryl, heteroaryl, -(aliphatic)aryl, -(aliphatic)heteroaryl, - -(heteroalipahtic)aryl, or -(heteroaliphatic)heteroaryl moiety, or is —(C═O)(CH)(R^{4B})NH(SO₂)R^{4C}, —SO₂R^{4B}, —(C═O)R^{4B}, —(C═O)N(R^{4B})₂, —(C═S)N(R^{4B})₂, or —(C═O)(CH₂)ₜ(C═O)NHR^{4B}, wherein each occurrence of R⁴B and R⁴c is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

28. The compound of claim 27, wherein R¹ is hydrogen, phenyl or methyl, R^{Z} is hydrogen or a solid support unit; R² is a substituted or unsubstituted alkyl or heteroalkyl moiety, or a substituted or unsubstituted aryl or heteroaryl moiety; and R^{4A} is —(C═O)(CH₂)t(C═O)NHR^{4B}, wherein R^{4B} is hydroxyl, t is 3, 4 or 5.

29. The compound of claim 1, wherein the compound has the structure: 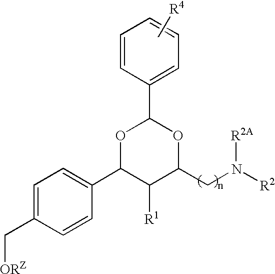
wherein R⁴ is —(CH₂)ᵣN(R^{4A})₂, —(CH₂)ᵣSR^{4A}, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or —(heteroaliphatic)heteroaryl moiety, or is —(C═O)(CH)(R^{4B})NH(SO₂)R^{4C}, —SO₂R^{4B}, —(C50 O)R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

30. The compound of claim 29, wherein R¹ is hydrogen, phenyl or methyl, RZ is hydrogen or a solid support unit; either or both of R² and R^{2A}, or R² and R^{2A} taken together with N, is a substituted or unsubstituted alkyl or heteroalkyl moiety, or a substituted or unsubstituted aryl or heteroaryl moiety; and R⁴ is —(CH₂)ᵣN(R^{4A})₂, —(CH₂)ᵣSR^{4A}, —(CH₂)ᵣOR^{4A}, —(CH₂)ᵣNR^{4A}C(═O), —(CH₂)ᵣ(C═O)N(R^{4A})₂, —S(O)₂R^{4A}, or is an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, —SO₂R^{4B}, —(C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

31. The compound of claim 29, wherein R^{4A} is —(C═O)(CH₂)ₜ(C═O)NHR^{4B}, wherein R^{4B} is hydroxyl, t is 3, 4 or 5.

32. The compound of claim 1, wherein the compound has the structure: 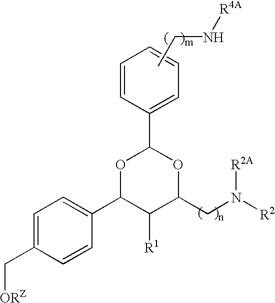
wherein each occurrence of R^{4A} is independently hydrogen, a protecting group, an aliphatic, heteroaliphatic, aryl, heteroaryl, -(aliphatic)aryl, -(aliphatic)heteroaryl, -(heteroalipahtic)aryl, or -(heteroaliphatic)heteroaryl moiety, or is —(C═O)(CH)(R^{4B})NH(SO₂)R^{4C}, SO₂R^{4B}, (C═O)R^{4B}, (C═O)N(R^{4B})₂, —(C═S)N(R^{4B})₂, or —(C═O)(CH₂)ₜ(C═O)NHR^{4B}, wherein each occurrence of R^{4B} and R^{4C} is independently hydrogen, a protecting group, hydroxyl, protected hydroxyl, or an alipahtic, heteroaliphatic, aryl, heteroaryl, -(aliphatic)aryl, -(aliphatic)heteroaryl, -(heteroalipahtic)aryl, or -(heteroaliphatic)heteroaryl moiety, wherein r and t are each independently 0-5,
whereby each of the foregoing aliphatic and heteroaliphatic groups is independently substituted or unsubstituted, cyclic or acyclic, linear or branched, and each of the foreoing aryl and heteroaryl groups is substituted or unsubstituted.

33. The compound of claim 32, wherein R¹ is hydrogen, phenyl or methyl, R^{Z} is hydrogen or a solid support unit; either or both of R² and R^{2A}, or R² and R^{2A} taken together with N, is a substituted or unsubstituted alkyl or heteroalkyl moiety, or a substituted or unsubstituted aryl or heteroaryl moiety; and R^{4A} is —(C═O)(CH₂)ₜ(C═O)NHR^{4B}, wherein R^{4B} is hydroxyl, and t is 3, 4 or5.

34. A pharmaceutical composition comprising:
a compound of any one of claims 1-33; and
a pharmaceutically acceptable carrier.

35. A method for inhibiting histone deacetylase activity comprising contacting a cell with a compound of any one of claims 1-33.

36. The method of claim 35, wherein the histone deacetylase is HDAC1 or HDAC6.

37. A method for treating cancer comprising:
administering to a subject in need thereof a therapeutically effective amount of a compound of any one of claims 1-33.

38. The method of claim 37, further comprising administering an additional therapeutic agent.
